SEQUENCE LISTING (3 sequences)

SEQ ID NO: 1 - DNA, 83 bp, Artificial Sequence
  Description of Artificial Sequence Synthetic oligonucleotide
ctgtctagacaaaaagtggtagtcctgtttcagctctcttgaagctgaaacaggactaccacggggatctgtggtctcataca

SEQ ID NO: 2 - DNA, 83 bp, Artificial Sequence
  Description of Artificial Sequence Synthetic oligonucleotide
ctgtctagacaaaaagcttctgctcgccaataaatctcttgaatttattggcgagcagaagcggggatctgtggtctcataca

SEQ ID NO: 3 - protein, 6 aa, Artificial Sequence
  Description of Artificial Sequence Synthetic 6xHis tag
HHHHHH

(19) United States
(12) Patent Application Publication
(10) Pub. No.: US 2012/0046329 A1
(43) Pub. Date: Feb. 23, 2012

(54) MODULATORS OF NOD1 AND NOD2 SIGNALING, METHODS OF IDENTIFYING MODULATORS OF NOD1 AND NOD2 SIGNALING, AND USES THEREOF

(75) Inventors: Gregory Paul Roth, Oviedo, FL (US); Ricardo Garcia Correa, San Diego, CA (US); Pasha Moeenuddin Khan, Jupiter, FL (US); John Christian Reed, Rancho Santa Fe, CA (US)

(73) Assignee: Sanford-Burnham Medical Research Institute

(21) Appl. No.: 13/207,325

(22) Filed: Aug. 10, 2011

Related U.S. Application Data
(60) Provisional application No. 61/500,105, filed on Jun. 22, 2011.
(60) Provisional application No. 61/372,383, filed on Aug. 10, 2010.

(51) Int. Cl. A61K 31/4184; C40B 30/02; C40B 30/00; A61P 3/10; A61P 31/00; A61P 25/28; A61P 9/00; C12N 9/50; A61P 29/00

(52) U.S. Cl. 514/395; 435/219; 506/8; 506/7

(57) ABSTRACT

Disclosed herein are compositions and methods relating to modulators of Nod-like Receptors NOD1 (NLRC1) and NOD2 (NLRC2) signaling. Further provided are methods of identifying modulators of Nod-like Receptors NOD1 and NOD2 activity. Further provided are compositions and methods for treating or preventing inflammation, including diseases associated with inflammation such as inflammatory bowel diseases (Crohn's disease, ulcerative colitis), pancreatitis, arthritis, asthma, psoriasis. Alzheimer's disease, cardiovascular disease (arteritis), diabetes, and sepsis.

This application claims benefit of U.S. provisional application No. 61/372,383, filed Aug. 10, 2010 and U.S. provisional application No. 61/500,105 filed Jun. 22, 2011 each of which is incorporated herein by reference.

### STATEMENT REGARDING FEDERALLY SPONSORED RESEARCH

This invention was made with government support under Grant Nos. 1 R03 MH084844-01, R03 MH084844, and AI-56324 from the National Institutes of Health (NIH). The government has certain rights in the invention.

### FIELD OF THE INVENTION

This application relates to methods and compositions for modulating nucleotide-binding oligomerization domain 1 (NOD1) and/or NOD2, also known as NLRC1 and NLRC2, respectively. In some embodiments, NOD1 modulation may induce nuclear factor κB (NF-κB) activation.

### BACKGROUND OF THE INVENTION

Humans are frequently challenged by an enormous diversity of microbes, and throughout evolution have developed efficient strategies to fight off eventual infections. Among these strategies, the innate immune system provides an early and effective response against pathogens. The success of this immune response relies on the recognition of conserved structures termed pathogen-associated molecular patterns, (PAMPs) commonly present in microbes but not in the host, by pattern recognition molecules (PRMs), acting as microbial sensors.

NLRs (NACHT and Leucine Rich Repeat domain containing proteins) constitute a prominent family of innate immunity proteins found in mammals. NLR-family proteins (NOD-Like Receptors [NLRs]) are components of innate immunity, constituting large families of related proteins that contain a nucleotide-binding oligomerization domain called NACHT and several leucine-rich repeat (LRR) domains involved in pathogen sensing (Ting et al., (2008) Immunity 28, 285-7; Mariathasan & Monack, (2007) Nat Rev Immunol 7, 31-40). Binding of pathogen-derived molecules to the LRRs is thought to induce conformational changes that allow NACHT-domain mediated oligomerization, thus initiating downstream signaling events, including activation of proteases involved in cytokine processing and activation. In this regard, the NACHT and LRRs are often associated with other domains that allow many of the NLRs to bind directly to pro-Caspase-1 (via CARD domains) or indirectly through adaptor proteins (via PYRIN domains). Caspase-1 belongs to the inflammatory group of Caspases, which cleave pro-Interleukin-1β (IL-1β), pro-IL-18, and pro-IL-33 (Salvesen, (2002) Essays Biochem 38, 9-19) in the cytosol, thus preparing them for secretion. Excessive activation of Caspase-1 can also induce cell death, either by apoptosis or by a variant recently termed “pyroptosis,” especially during host responses to pathogens (for review, sec (Ting et al., (2008) Nat Rev Immunol 8, 372-9)).

The nucleotide-binding oligomerization domain (NOD) proteins NOD1 (NLRC1) and NOD2 (NLRC2) are some of the major cytosolic sensors of PRMs for bacterial infection. NOD1 (also termed as CARD4 or CLR7.1) and NOD2 (also termed as CARD15; CD; BLAU; IBD1; PSORAS1; CLR16.3) are members of the Nod-like receptor (NLR) family, which share structural similarity with a subset of plant disease-resistance (R) proteins involved in the hypersensitive response against plant pathogens. The NLR proteins display (a) a C-terminal leucine-rich repeat (LRR) domain that is involved in recognition of conserved microbial patterns or other ligands (‘danger signals’); (b) a centrally located nucleotide-binding NACHT domain that binds nucleotide triphosphates and mediates self-oligomerization, which is essential for NLR activation; and (c) a N-terminal effector domain, which is responsible for the interaction with adaptor molecules that result in signal transduction.

The NOD proteins participate in the signaling events triggered by host recognition of specific motifs (mostly, muropeptides) in bacterial peptidoglycan (PG). Upon activation, NODs induce activation of NF-κB, a central regulator of immune response, inflammation, and apoptosis. NOD2 is a general bacterial sensor that participates in the innate immunity against Gram-positive bacteria (S. pneumoniae, L. monocytogenes), Gram-negative bacteria (S. typhimurium) and mycobacteria (M. tuberculis), while NOD1 recognizes mainly Gram-negative bacteria (E. coli, Chlamydia, H. pylori). Prior studies have shown the muramyldipeptide (MDP), a PG component, stimulates NOD2 activation in cells, while Ala-γ Glu-diaminopimelic acid (γ-tri-DAP) stimulates NOD1, thus providing convenient, synthetic ligands for activating the proteins in intact cells.

NOD1 and NOD2 act through the NOD signaling pathway. NOD1 and NOD2 physically associate with RICK (Ripk2/Rip2/CARDIAK), a CARD-containing protein kinase, through homophilic CARD-CARD interactions. Once RICK is recruited, it interacts with the IKK subunit IKKγ (also called NEMO), as well as other proteins (such as members of the IAP family), promoting NEMO modification with Lysine 63-linked polyubiquitin chains (which are not substrates for the proteasome), resulting in activation of the IκB kinases (IKKs) that phosphorylate the NF-κB inhibitor IκBα, targeting it for Lysine 48-linked polyubiquitination and proteasome-dependent degradation (Abbott, D. et al., Curr Biol, 14: 2217-2227, 2004; Yoo, N. J. et al., Biochem Biophys Res Commun, 299: 652-658, 2002; Hasegawa, M., et al. Embo J, 2007). After IκBα is degraded, free NF-κB translocates into the nucleus, where it drives the transcription of κB-containing genes (Li, Q. and Verma, I. M. Nat Rev Immunol, 2: 725-734, 2002, Perkins, N. D. Nat Rev Mol Cell Biol, 8: 49-62, 2007). Over-expression of NOD1, NOD2, or RICK is able to induce NF-κB activation (Inohara, et al., J Biol Chem, 274: 14560-14567, 1999; Bertin, J. et al., J Biol Chem, 274: 12955-12958, 1999; Ogura, Y. et al., J Biol Chem, 276: 4812-4818, 2001). The NOD proteins are also involved in the Interferon Response Factor (IRF) pathway and the AP-1 pathway (including the stress kinase pathway). Examples include the following pathways:
NOD→Rip2→IAP→UBC13→TAB/TAK→IKK→IκBα→NF-κB
NOD→Rip2→XIAP→TAB/TAK→IKK→IκBαΘNF-κB
NOD→Rip2→IAP→UBC13→TAB/TAK→JNK→AP-1
NOD→Rip2→IAP→UBC13→TAB/TAK→p38 MAPK→AP-1
NOD→MAVS→TRAF3→TBK1→IRFs→Interferon

In addition to activating inflammatory caspases and NF-κB, certain NLR family members, including NOD1 and NOD2, stimulate additional innate immunity effector mechanisms. For example, NOD1 and NOD2 stimulate autophagy, which is useful for elimination of intracellular microbes by lysosome-dependent destruction. NOD1 and NOD2 also activate members of the IRF family of transcriptions factors involved in the type I interferon response, which is important in host defense against viruses. Additionally, NOD1 and NOD2 stimulate activation of “stress kinases”, leading to activation of Jun N-terminal kinases (JNKs) and p38 Mitogen-Activated Protein Kinase (p38 MAPK).

Mutations in NOD1 and NOD2 are associated with a number of human inflammatory disorders, including Crohn's disease (CD), Blau syndrome, early-unset sarcoidosis, and atopic diseases, which cause NF-κB constitutive activation (Carneiro, L. et al., J Pathol, 214: 136-148, 2008; Franchi, L. et al., Cell Microbiol, 10:1-8, 200819). In diseases such as asthma or inflammatory bowel disease, there is a change of NOD1 expression to certain splice variant isoforms, which lead to abnormal inflammation (Carneiro. L. et al., J Pathol, 214: 136-148, 2008). In addition, intestinal macrophages of CD patients overproduce NF-κB targets, including the pro-inflammatory cytokines tumor necrosis factor α (TNFα) and the interleukins IL-1β and IL-6 (Lala, S. et al., Gastroenterology, 125: 47-57, 2003; Maeda, S. et al., Science, 307: 734-738, 200521). Notably, the fact that NOD2 has been identified as the first susceptibility gene for Crohn's disease (Maeda, S. et al., Science, 307: 734-738, 200521; Hugot, J. P. et al., Nature, 411: 599-603, 2001) suggests intriguing interconnections between bacterial sensing and chronic inflammatory diseases.

A need exists for chemical modulators of NLR family proteins such as NOD1 for elucidating the roles of these proteins in achieving a proper balance of innate immunity responses and for exploring whether novel therapeutic interventions can be developed based on targeting this class of proteins. There is also a need for modulating NOD1 induced NF-κB activation, for example, for treating various inflammatory and infectious disorders. For example, there is a need for inhibiting NOD1-induced NF-κB activation selectively over NF-κB activation induced by NOD2 or tumor necrosis factor-α (TNF-α). Also, there is a need for inhibiting NOD1-induced stress kinase activation and IRE activation.

### SUMMARY OF THE INVENTION

In accordance with the purpose of this invention, as embodied and broadly described herein, this invention relates to compositions and methods relating to identification and use of modulators of NOD1 and/or NOD2. Further provided are compositions and methods for treating or preventing, for example, inflammation, including diseases associated with inflammation such as inflammatory bowel disease, Crohn's disease, ulcerative colitis, arthritis, psoriasis, Alzheimer's disease, cardiovascular disease, diabetes, and sepsis (fulminant infection).

Provided herein are methods for modulating NOD1, NOD1-induced NF-κB activation, NOD1-induced stress kinase activation, and NOD1-induced interferon response, comprising contacting NOD1 with a compound disclosed herein. As used herein, modulating refers to inhibiting or increasing NOD1 biological activity, which optionally results in a modulation of NOD1-induced NF-κB activation or/and NOD-1-induced IRF activation and NOD1-induced stress kinase activation. In one embodiment, the method is for inhibiting NOD1-induced NF-κB activation. In another embodiment, the method is for increasing NOD1-induced NF-κB activation. In still other embodiments, the method is for modulating NOD1-induced IRF activation, thus impacting type I interferon responses, and stress kinase activation. Stress kinase activation can be measured by a variety of methods, including (a) in vitro kinase assays and (b) using phosphor-specific antibodies that detect phosphorylation events on JNKs and p38MAPK associated with activation of these protein kinases. In yet other embodiments, the method is for modulating NOD1-induced autophagy stimulation or NOD1-induced inflammatory activation of caspase-1, 4, or 5.

Also provided herein are methods for treating diseases that can be treated by modulating NOD1 and/or NOD1-induced NF-κB activation comprising administering a compound disclosed herein in an amount that is effective for modulating NOD1 and/or NOD1-induced NF-κB activation to a patient in need of such treatment. In one embodiment, the amount of the compound administered is effective for inhibiting NOD1 and/or NOD1 induced NF-κB activation. In another embodiment, the amount of the compound administered is effective for increasing NOD1 biological activity and/or NOD1-induced NF-κB activation. In certain embodiments, the treatment methods further comprise administering another agent as disclosed in the Combination Therapeutics section below. Analogous embodiments apply as concerns the ability of NOD1 to activate IRF family transcription factors involved in the type I interferon response, and the ability to NOD1 to stimulate autophagy, and the ability of NOD1 to activate stress kinases involved in inflammatory responses (e.g. JNKs, and p38 MAPK).

Also provided herein are pharmaceutical compositions comprising a pharmaceutically acceptable carrier or a pharmaceutically acceptable excipient and an amount of a compound effective for modulating NOD1 and/or NOD1 induced NF-κB activation. In one embodiment, the amount of the compound is effective for inhibiting NOD1 and/or NOD1 induced NF-κB activation. In another embodiment, the amount of the compound is effective for increasing NOD1 biological activity and/or NOD1 induced NF-κB activation. In certain embodiments, the pharmaceutical compositions are contemplated to further comprise an agent as disclosed in the Combination Therapeutics section below.

In one aspect, the compound utilized herein is of Formula A:

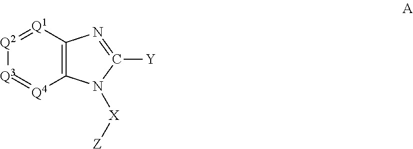

wherein,
Q¹ is N or CR_{Q1},
Q² is N or CR_{Q2},
Q³ is N or CR_{Q3},
Q⁴ is N or CR_{Q4}, provided that at least one of Q¹-Q⁴ is not N,
R_{Q1} is H, R⁴² or R⁵¹;
R_{Q2} is H, R¹, R⁴² or R⁵²:
R_{Q3} is H, R², R⁴² or R⁵³;
R_{Q4} is H or R⁴²:
Z is:

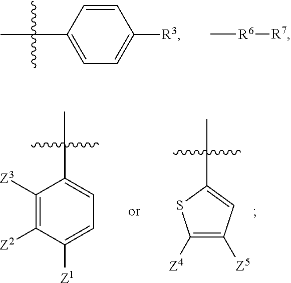

and
R¹ to R³, R⁴², R⁵¹ to R⁵³, X, Y, and Z¹ to Z⁵ are as defined in any aspect or embodiment herein.

In one aspect utilized herein are compounds having the structure of Formula I:

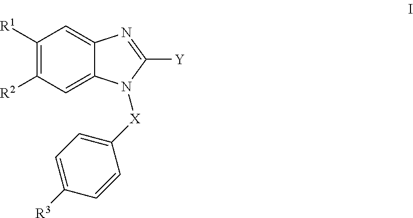

or the pharmaceutically acceptable salt or ester thereof,
wherein R¹ and R² are independently hydrogen or C₁-C₃ alkyl;
R³ is hydrogen, C₁-C₃ alkyl, C₁-C₃ alkenyl, C₁-C₃ alkoxy, or halogen; X is —(CH₂)₁₋₃—, —(CH₂)₁₋₃O—, —(CH₂)₁₋₂O(CH₂)₁₋₂—, —SO₂—, —(CH₂)₁₋₃SO₂—, —C(O)—, —(CH₂)₁₋₃C(O)—, —(CH₂)₁₋₂C(O)(CH₂)₁₋₂—, or —NH—, —N(R⁴)—;
R⁴ is C₁-C₃ alkyl; and Y is hydrogen, amino, C₁-C₃ alkylamino, thio, C₁-C₃ alkylthio, C₁-C₃ alkyl, C₁-C₃ alkoxy, or halogen.

Also utilized herein are compounds of Formula II:

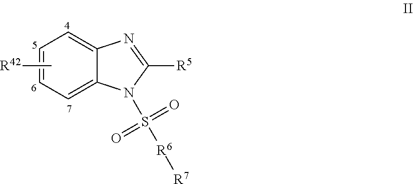

wherein:
one of positions 4, 5, 6 or 7 can optionally be aza substituted;
R⁴² is hydrogen, C₁-C₃ alkyl, C₁-C₃ alkenyl, C₁-C₃ alkoxy, C₁-C₃ haloalkyl, C₁-C₃ alkylamino, amino, aminoacetyl, nitro, nitrile, halogen, —CO₂R²¹ or —C(O)N(R²²)(R²³);
R²¹ is hydrogen or C₁-C₃ alkyl;
R²² and R²³ can be independently hydrogen or C₁-C₃ alkyl;
R⁵ is hydrogen, amino, thio, C₁-C₃ alkylthio, C₁-C₃ alkoxy, hydroxyl, —N(R²³)(R²⁴), C₁-C₃ alkylamino, C₁-C₃ alkylaminoacetyl, or —NH(CH₂)₁₋₃—;
R²³ and R²⁴ independently are hydrogen or C₁-C₃ alkyl;
R⁶ is present or absent, if present R⁶ can be —(CH₂)₁₋₃—;
R⁷ is aryl, heteroaryl, cycloalkyl or heterocyclyl.

In one embodiment, the compound utilized according to the present technology is of Formula III:

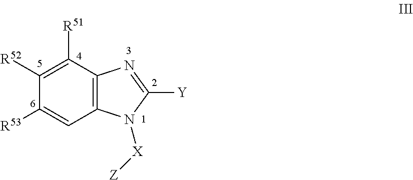

or a pharmaceutically acceptable salt thereof,
wherein R⁵¹ is H, C₁-C₆ alkyl, C₃-C₆ cycloalkyl, or NH₂;
R⁵² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or fluoro;
R⁵³ is H, C₁-C₆, alkyl, C₁-C₆ alkyl substituted with fluoro, C₃-C₆ cycloalkyl, C₃-C₆ cycloalkyl substituted with fluoro, halo, CO₂H, or a carboxyl ester;
Y is NH₂, H, NH(CH₂)₃OH, CH₃, or —CH₂NHCHO;
X is SO₂, CO, —CH₂—, or —CH₂CH₂CO—;
Z is:

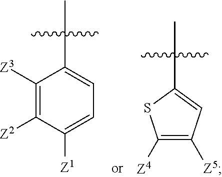

Z¹ is H, NO₂, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₁-C₆ alkoxy substituted with fluoro, C₃-C₆ cycloalkoxy, C₃-C₆ cycloalkoxy substituted with fluoro, halo, or

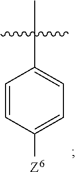

Z⁶ is H, C₁-C₃ alkoxy, cycloalkoxy, or halo; Z₂ is H, C₁-C₆ alkyl, C₁-C₆ alkyl substituted with fluoro, C₃-C₆ cycloalkyl, C₃-C₆ cycloalkyl substituted with fluoro, alkoxy, cycloalkoxy, or halo, or Z¹ and Z² together with the carbon atoms they are attached to form a 5-7 membered ring;
Z³ is H or halo;
Z⁴ and Z⁵ are independently H or halo, or Z⁴ and Z⁵ together with the carbon atoms they are attached to form a 5-7 membered ring.

Preferred embodiments of utilized compounds of Formula III are disclosed below. In one embodiment, R⁵³ is H. In another embodiment, R⁵² and R⁵³ are H. In another embodiment, R⁵¹ and R⁵³ are H. In another embodiment, R⁵¹ and R⁵² are H. In another embodiment, R⁵¹, R⁵² and R⁵³ are H.

In another embodiment, X is SO₂.

In another embodiment, Y is NH₂.

In another embodiment, Z is:

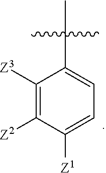

In another embodiment, Z¹ is H, NO₂, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₁-C₆ alkoxy substituted with fluoro, C₃-C₆ cycloalkoxy, C₃-C₆ cycloalkoxy substituted with fluoro, or halo. In another embodiment, Z² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or halo. In another embodiment, Z³ is H.

In another embodiment, Z is:

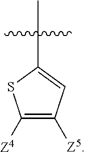

In another embodiment, R⁵¹, R⁵² and R⁵³ are H; Y is NH₂; X is SO₂; Z is:

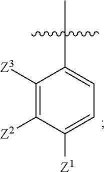

Z¹ is H, C₁-C₆ alkyl, C₁-C₆ alkyl substituted with fluoro, C₁-C₆ alkoxy, or halo; Z² is H, C₁-C₆ alkyl, C₁-C₆ alkoxy, or halo, or Z¹ and Z² together with the carbon atoms they are attached to form a 5 membered ring containing carbon ring atoms; and Z³ is H.

In another embodiment, Z¹ is H, methyl, isopropyl, trifluoromethyl, methoxy, or chloro.

In another embodiment, Z² is H, propyl, tertiary butyl, methoxy, or halo.

In another embodiment. R⁵¹, R⁵² and R⁵³ are H; Y is NH₂; X is SO₂; Z is:

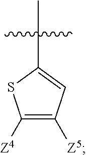

and
Z⁴ and Z⁵ together with the carbon atoms they are attached to form an aromatic ring.
In another embodiment. R⁵³ is H; at least one of R⁵¹ and R⁵² is a non hydrogen substituent; Y is NH₂; X is SO₂; Z is:

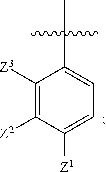

Z¹ is C₁-C₆ alkyl or C₃-C₆ cycloalkyl; and Z² and Z³ are H.

In another embodiment, R⁵¹, R⁵² and R⁵³ are H; Y is NH₂; X is SO₂; Z is:

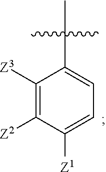

and

### Z₂ and Z₃ are H.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIG. 1 demonstrates that compound 1 (also referred to as CID-1088438) specifically inhibits NOD1-dependent signaling pathways.

FIG. 1(A) PMA-differentiated THP.1-cells containing NF-κB-driven SEAP (10⁵ cells/well in 96-well plates) were cultured with or without 5 μM CID-1088438 or the negative control CID-44229067 with the various TLR inducers: 0.5 μg/ml Pam3CSK4 (TLR1/TLR2), 5×10⁷ cells/ml HKLM (TLR2), 1 μg/ml FSL-1 (TLR6/2), 0.5 g/ml LPS (TLR4), 0.5 μg/ml Flagellin (TLR5), 1 μg/ml ssRNA40 (TLR8), 5 μg/ml γ-tri-DAP (NOD1), 5 μg/ml MDP (NOD2) or 5 ng/ml TNF-α. After 24 h incubation, SLAP activity in culture supernatants was measured, expressing data as percentage relative to treatment with inducer only (indicated as 100%; mean±SEM, n=2).

FIG. 1(B) 697 cells stably containing a NF-κB luciferase reporter gene (10⁵ cells/well in 96-well plates) were cultured with or without 10 μM compound 1 or CID-44229067, in combination with the respective inducers: 20 μg/ml γ-tri-DAP, 100 ng/ml BAIT or 5 μM ODN2006 (TLR9). Luciferase activity was measured 24 h later (mean±SD; n=3).

FIG. 1(C) 293T cells, stably expressing luciferase reporter gene driven by IFN responsive elements (10 cells/well in 96-well plates), were cultured with or without 5 μM CID-1088438 or CID-44229067, in combination with the respective inducers: 10 μg/ml γ-tri-DAP (NOD1), 1 μg/ml poly(I:C) with lipid transfection (LyoVee) (RIG-I/MDA-5), 1 μg/ml Poly(dA:dT) (LyoVee) (IRF3) or Sendai Virus (classical IRF3 inducer). Luciferase activity was measured after 24 hrs (mean±SD; n=4).

FIG. 1(D) RAW264.7 cells (5×10⁴ cells/well in 96-well plates) were treated with 5 μM of compound 1 or CID-44229067, then stimulated with 100 ng/ml monosodium urate (MSU), 1 μg/ml poly(dA:dT) or 1 μg/ml LPS plus 5 mM ATP, after LPS pretreatment (induction of pro-IL-1β synthesis), or infected with S. typhimurium at multiplicity of infection (MOI) of 20 and 200 bacteria per mammalian cell. Supernatants were collected after either 2 hrs (Salmonella infection) or 4 hrs (all others) and IL-1β levels were quantified by ELISA (mean+SD; n=3).

FIG. 1(E) Dendritic cells (DCs) were activated with either 5 μg/ml γ-tri-DAP or 100 ng/ml LPS, in the presence or absence of 15 μM CID-1088438. After 24 hr, flow cytometry analysis was performed for CD83, CD86 and HLA-DR markers. Representative data from one donor are shown (n=3).

FIG. 1(F) Expression of prototypical NF-κB target genes in primary monocyte-derived DCs. Cells were treated with either 5 μg/ml γ-tri-DAP or 100 ng/ml LPS, in the presence or absence of 15 μM CID-1088438. After 4 hr, relative mRNA expression of IL-1β, IL-6, TNFα and NOD1 were determined by quantitative PCR, Results were normalized according to β-actin levels (mean±SEM of three donors).

FIG. 1(G) Caspase-dependent cell death (pyroptosis) is not affected by NOD1 inhibitory compounds. RAW264.7 cells (5×10⁴ cells/well into 96-well plate) were treated with 5 μM of CID-1088438 or CID-44229067 alone, or also infected with S. typhimurium at multiplicity of infection (MOI) of 20 and 200 bacteria per mammalian cell. Cell viability was analyzed two hours after Salmonella infection by measuring ATP levels (Cell TiterGlo, Promega). Percentage viability was calculated according to the ATP levels of respective non-infected cells (standardized as 100% viable). Values presented are averages of two replicates (+SEM).

FIG. 2 illustrates the mechanisms of chemical inhibitors of NOD1.

FIG. 2(A) HEK 293T-NF-κB-luciferase cells were transfected with plasmids encoding NOD1, RIP2, XIAP, or GFP in various combinations. After 24 hr, cells were cultured with or without 3.5 μg/ml γ-tri-DAP, 10 μM of CID-1088438, or combinations of these reagents for 24 hrs before measuring luciferase activity normalizing data to GFP-transfected cells (100%) (mean±SD, n=4).

FIG. 2(B) 1D 1H-NMR spectra were collected for compound 1 (50 μM) in the absence (upper spectrum) and presence (lower spectra) of 5 μM of His6-Flag-NOD1, His6-Bcl-XL, or His6-Bid purified proteins, respectively, compound 1-derived proton signal intensity (arrows) is only suppressed in the presence of NOD1, thereby demonstrating direct interaction between ligand and protein.

FIG. 2(C) Purified His6-Flag-NOD1, His6-Bcl-XL, or His6-Bid proteins (≈0.4 μg) were pre-incubated for 60 minutes with 20 μM compound 1 or CID-44229067 (DMSO as control). Ni-NTA agarose beads were then added and the mixture was incubated overnight at 4° C. Ni/NTA pull-down was performed and samples were analyzed by immunoblotting using anti-His antibody (top). Quantification of proteins on blots was performed, normalizing data relative to DMSO control (bottom).

FIG. 2(D) MCF-7 cells stably expressing His6-FLAG-NOD1 or His6-LAGNOD2 were cultured with 5 μM of CID-1088438 or CID-44229067 and 10 μg/ml γ-tri-DAP alone or with 1 μM MG-132. After 16 hr, cells were lysed and equal amounts of protein samples were pulled down using Ni/NTA resin beads. Total protein lysates (30 μg) and Ni/NTA-bound proteins were analyzed by immunoblotting using anti-FLAG antibody.

FIG. 2(E) MCF-7 cells stably expressing His6-FLAG-NOD1 were cultured with 5 μM of CID-1088438 or CID-44229067 alone or combined with 5 μg/ml γ-tri-DAP. After 24 hr, cytosolic and membrane subcellular fractions were isolated and analyzed (10 μg protein) by immunoblotting using antibodies specific for γ-tubulin (cytosolic marker), pan-cadherin (plasma membrane marker). NOD1 (using anti-FLAG antibodies) and RIP2. Short (s.e.) versus long (l.e.) exposures of blots are presented for some results. All data are derived for a single blot. Intervening lanes were graphically excised (vertical line) for efficiency of presentation.

FIG. 3 demonstrates that the NOD1 inhibitory compound does not compete for ATP binding.

FIG. 3(A) Various concentrations of recombinant 6His-Flag-Nod1 or GST protein were incubated with 10 nM FITC-conjugated ATP in FPA buffer (20 mM HEPES buffer containing 0.005% Tween 20).

FIGS. 3(B-D) Various concentrations of compound 1, the negative control, or ATP (positive control) were incubated with recombinant (B) 6His-FLAGNOD1 (50 nM), (C) GST-NLRP1 (100 nM) or (D) GST-Hsp70 (20 nM) for 2 min, then 10 nM FITC-conjugated ATP in FPA buffer was added. Fluorescence polarization was measured after 10 min. Values are expressed in milli-Polars (mP), presented as averages of two replicates (+SEM).

FIG. 4 demonstrates the effects of compounds on NOD1 ubiquitination status. HEK 2931 cells from 10-cm plates were co-transfected with 6.5 μg of each plasmids encoding hemaglutinin (HA)-tagged ubiquitin. VSV-tagged RIP2 and 6×His-FLAG-tagged NOD1 then further treated after 1 day with 5 μM of compound 1 or CID-44229067. After 24 h treatment, cells were lysed and equal amounts of protein were immunoprecipitated (I.P.) using anti-FLAG beads. Total protein lysates (≈50 μg) and FLAG immunoprecipitates were analyzed by immunoblotting. Actin levels were assessed as loading control (left). FLAG immunoprecipitates were additionally analyzed by immunoblotting using K48- and K63-specific ubiquitin antibodies (right). NOD1 and RIP2 proteins are indicated (arrows).

FIG. 5 demonstrates that the inhibitory compound docs not block RIP2 binding to NOD1, HEK 2931 cells from 10-cm plates were co-transfected with 12.5 μg of 6×His-FLAGNOD1 plus 12.5 μg of Myc-NOD1 or Myc-RIP2 expression vectors, and further treated with 5 μM of prototypical NOD1 inhibitor (compound 1) or CID-44229067. After 24 h treatment, cells were lysed and equal amounts of protein were immunoprecipitated (I.P.) using anti-FLAG beads. Total protein lysates (≈50 μg) and FLAG immunoprecipitates were analyzed by immunoblotting with anti-FLAG and anti-Myc antibodies. Actin levels were assessed as loading control. NOD1 and RIP2 protein bands are indicated (arrows).

FIG. 6 demonstrates the effects of compounds on NOD1 protein interactions. HEK 293T cells from 10-cm plates were co-transfected with 6.5 μg of each plasmids encoding hemaglutinin (HA)-tagged ubiquitin, VSV-tagged RIP2, Myc-tagged NOD1 plus 6His-FLAG-tagged NOD1 or 6His-FLAG-tagged NOD2 (control), and further treated with 5 μM of prototypical NOD1 inhibitor (compound 1) or CID-44229067. After 24 h treatment, cells were lysed and equal amounts of protein were pulled-down using Ni/NTA agarose beads. Total protein lysates (≈60 μg) and Ni/NTA-bound proteins were analyzed by immunoblotting. Actin levels were assessed as loading control. Active compound 1 causes more 6His-FLAG-NOD1 protein (but not NOD2) pull-down with Ni/NTA resin, possibly reflecting a conformational change. Interaction of over-expressed 6His-FLAG-NOD1 with Myc-NOD1, RIP2, and SGT-1 was not inhibited by active compound.

FIG. 7 demonstrates that compound 1 does not affect cellular compartmentalization of NOD2. MCF-7 cells stably expressing 6His-FLAG-NOD2 were treated with 5 μM of compound 1 or CID-44229067 alone or combined with 5 μg/ml MDP. After 24 h treatment, cytosolic and membrane subcellular fractions were isolated and analyzed (10 μg protein) by immunoblotting using antibodies specific for α-tubulin (cytosolic marker), pan-cadherin (plasma membrane marker), NOD2 (using anti-FLAG antibodies) and RIP2. Short (s.c.) versus long (l.e.) exposures of blots are presented for some results. All data are derived for a single blot. Intervening lanes were graphically excised (vertical line) for efficiency of presentation. Note that changes on RIP2 protein levels follow similar pattern when compared to α-tubulin levels, indicating no major differences after normalization.

FIG. 8 shows the NF-κB dependent luciferase activity using increasing amounts of transduced 293T cells. Gamma-tri-DAP (0.5 or 1.0 μg/ml) was added to NF-κB-luciferase containing 293T cells in DMEM medium without Fetal Bovine Serum (FBS) and Phenol Red, 0.5% DMSO. Luminescence intensity (counts per second, CPS) was measured 18 hours post-treatment using Britelite Assay System (Perkin-Elmer) in a LJL Analyst. Solid curves represent best-tit in linear regression (n=24).

FIG. 9 shows the NF-κB-dependent luciferase activity over time of treatment. Gamma-tri-DAP (2.0 μg/ml) was added to NF-κB-luciferase containing 293T cells in DMEM medium without Fetal Bovine Serum (FBS) and Phenol Red, 0.5% DMSO. Luminescence intensity (counts per second, CPS) was measured at various times after treatment, using Britelite Assay System (Perkin-Elmer) and a LJL Analyst. Solid curve represent best-fit in non-linear regression (using triplicates).

FIG. 10 shows the NF-κB-dependent luciferase activity using increasing amounts of NOD1-specific inducer γ-tri-DAP. Different concentrations of inducer were added to a fixed number of NF-κB-luciferase containing 293T cells (104 cells/well) as described in FIG. 8. Luminescence intensity (counts per second, CPS) was measured 16 hours post-treatment. Solid curve represents best-fit using non-linear regression (n=47). Maximum luciferase activity (Bmax)=5.045E+06; Kd=0.7559.

FIG. 11 shows the statistical analysis of NOD1 cell-based primary assay. Transduced 293T cells (104 cells/well) were treated (red symbols) or not treated (blue symbols) with 0.75 μg/ml γ-tri-DAP for 16 hours, in DMEM without Fetal Bovine Serum (FBS) and Phenol Red, 0.5% DMSO (total of 50 μl per well). After incubation, twenty microliters of luciferase substrate were added (Britelite™ Assay System, Perkin-Elmer) per well, and 10 minutes later luminescence measurements were performed a LJL Analyst plate reader. The data represent mean values+standard deviations, calculated for both groups, and are representative of 3 independent experiments (11A, 11B and 11C) performed on different days.

FIG. 12 shows the 3D Scatter plot of NOD1 LOPAC screening results. Data represent the percentage of luciferase inhibition (z-axis), relative to control wells, by compound used in respective location (well position, x-axis; plate number, y-axis). Luciferase assay was performed as described in FIG. 11, using a total of four 384-well plates. Compounds were loaded into cell suspensions at 4 μM final and pre-incubated for one hour at room temperature. Next, cell induction was performed using 0.75 μg/ml γ-tri-DAP, followed by 16 hours of incubation at 37° C., 10% CO₂ incubator. Luminescence measurement followed as described in FIG. 11. Negative (0% inhibition, middle) and positive (100% inhibition, top) controls (light symbols), as well as putative NF-κB inhibitors and agonists, are indicated.

FIG. 13 shows NF-κB luciferase activity using increasing amounts of DMSO. Two different concentrations of NOD1-specific inducer (γ-tri-DAP) were tested with a fixed number of NF-κB-luciferase 293T cells per well (104 cells/well), as described in FIG. 8. Luminescence intensity (counts per second, CPS) was measured 16 hours after induction using Britelite Assay System (Perkin-Elmer) with a LJL Analyst (mean±std dev; n=22).

FIG. 14 shows results from a NOD1 secondary assay. (A) Optimization of cycloheximide levels. Stably transfected MCF7-NOD1 cells were induced with or without NOD1- or NOD2-specific inducers (γ-tri-DAP or MDP, respectively) for 24 hours, in the presence of increasing amounts of cycloheximide as an IL-8 releasing adjuvant. (B) Optimization of γ-tri-DAP levels. MCF7-NOD1 cells were induced for 24 hours with γ-tri-DAP, with or without 1.5 μg/ml cycloheximide. (C) Time-course of γ-tri-DAP treatment. MCF7-NOD1 cells were induced for different periods of time (as indicated) with 5.0 μg/ml γ-tri-DAP plus 1.5 μg/ml cycloheximide. IL-8 analysis was performed using Human IL-8 ELISA kit (BD Biosciences). All data points were performed in triplicates (mean±SD).

FIG. 15 shows the statistical analysis of NOD2 primary assay. Transduced 293T cells (10⁴ cells/well) were cultured at 37° C., 10% CO₂ incubator for 16 hours, in DMEM without Fetal Bovine Serum (FBS) and Phenol Red, 0.5% DMSO (total of 50 μl per well). After incubation, twenty microliters of luciferase substrate were added (Britelite™ Assay System, Perkin-Elmer) per well, and 10 minutes later luminescence measurements were performed using LJL Analyst. Mean values and standard deviations were calculated for both groups (inset), and the Z′ factor was calculated.

FIG. 16 shows the 3D Scatter plot of NOD2 LOPAC screening. Data represent the percentage of luciferase inhibition (z-axis), relative to control wells, by compound used in respective location (well position, x-axis; plate number, y-axis). Luciferase assay was performed as described in FIG. 15, using a total of four 384-well plates. Compounds were loaded into cell suspensions at 5 μM final and incubated for one hour at room temperature. Next, cell induction was performed using 0.75 μg/ml γ-tri-DAP, followed by 16 hours of incubation for 16 hours at 37° C., 10% CO₂. Luminescence measurements followed as described in FIG. 15. Negative (0% inhibition, middle) and positive (100% inhibition, top) controls (light symbols), as well as putative NF-κB inhibitors and agonists, are indicated.

FIG. 17 shows the general triage used to prosecute actives in NOD1 and NOD2 primary assays, which then “tri”-furcate into NOD1 selective, NOD2 selective and NOD1/2 dual selective inhibitors. The right hand branch in FIG. 17 at the “Specificity” branchpoint” represents NOD1 selective inhibitors to follow up.

FIG. 18 shows the flow of the assay identifying NOD1, NOD2, and TNFα modulators.

FIG. 19 shows the relationship between NOD1, NOD2 and TNFα modulators. FIG. 19 also shows assay set up for determining the relationship between the modulators that are specific to NOD1, NOD2 or both.

FIG. 20 shows the results from assays using NOD1, NOD2, TNFα, and alamar blue cytotoxicity. At this stage, the alamar blue cytotoxicity assay was multiplexed in dose response with the TNFα assay. The assays were performed using IL-8 ELISA methodology using MCF7 cells over expressing NOD1. The assay was also performed by subjecting the cells to NOD1, NOD2, or NOD-nonspecific substances. FIG. 20A shows the results after the cells were treated with γ-tri-DAP. FIG. 20B shows the results after the cells were treated with MDP-LD. FIG. 20C shows the results after the cells were treated with TNFα.

FIG. 21 shows the results of the NF-κB luciferase assay. FIG. 21A shows the results from the assay using DAP induction. FIG. 21B shows the results from the assay using Dox induction. FIG. 21C shows the results from the assay using PMA induction.

FIGS. 22A-22D show that XIAP is required for induction of cytokine production by NOD ligands. (A and B) HCT 116 XIAP−/− (WT=Wild-Type) and XIAP−/− cells (KO=knock-out) (A) or DLD-1 XIAP−/− or XIAP−/− cells (B) were stimulated with MDP (20 μg/mL), DAP (20 μg/mL), TNF-α (5 ng/mL), or left untreated for 24 h. Cell free supernatants were collected after centrifugation and analyzed for IL-8 secretion by ELISA. Data represent means±SD of three independent experiments (pg/mL). (C) Reduced expression of NOD ligand-inducible genes in XIAP-deficient cells. HCT116 XIAP−/− (white bars) and XIAP−/− (black bars) were stimulated for 1 h with various NF-κB inducers: 20 μg/mL γTri-DAP, 20 μg/mL MDP-LD, or 10 ng/mL TNF-α. RNA was isolated and relative levels of IκBα and IL-8 mRNAs were measured by Q-RT-PCR, normalized relative to 18S rRNA, expressed as relative levels compared with unstimulated cells (mean value=1), and presented as mean+std dev of triplicate determinations performed in at least two independent experiments, (D) HCT116 XIAP−/− cells (KO) were transfected with FLAG-XIAP-encoding plasmid or empty FLAG-plasmid, then stimulated 24 h post transfection with MDP (20 μg/mL), γTri-DAP (20 μg/mL), TNF-α (5 ng/mL), or left untreated. As a control, HCT116 XIAP−/− (WT) were similarly stimulated. NF-κB reporter gene activity was measured after 24 h using the Dual Luciferase assay method. Normalized values represented mean±SD (n=3). (Inset) Lysates from the cells were prepared, normalized for total protein content, and analyzed by immunoblotting using anti-XIAP antibody. Reprobing blot with anti-beta-Actin antibody confirmed equal loading. (E) XIAP deficiency selective impacts NOD-mediated NF-κB activation. HEK293T cells containing a stably integrated NF-κB-luciferase reporter gene were infected with XIAP shRNA (KD=knock-down) (white bars) or scrambled control (CNTL) (black bar) lentiviruses. After 24 h, cells were stimulated with 10 μg/mL MDP-LD (MDP), 5 μg/mL γTri-DAP (DAP), 0.2 μg/mL doxorubicin (DOX), 10 ng/mL PMA/ionomycin (PMA), or 2 ng/mL TNF-α. NF-κB activity was measured 24 h later by luciferase activity, and data were expressed as fold-induction relative to control unstimulated values for each cell line (mean value=1) and represent mean±std dev of triplicates performed in at least two independent experiments. Inset shows immunoblot analysis of lysates from the cells (100 μg total protein) using anti-XIAP (Top) and anti-beta-actin antibodies (Bottom).

FIGS. 23A-23H show that NF-κB activity induced by over-expression of NOD1 or NOD2 requires XIAP. (A and B) HCT116 XIAP−/− (WT) and XIAP−/− (KO) cells were seeded into 96-well plates at 2×104 cells per well. The next day cells were transfected with various amounts of plasmid DNA encoding Myc-NOD1 (A) or Myc-NOD2 (B), along with a fixed amount of NF-κB-Firefly luciferase and TK promoter-driven Renilla luciferase plasmids. NF-κB activity was measured 24 h posttransfection, normalizing Firefly relative to Renilla luciferase activity to determine relative levels of NF-κB activity (Firefly LUC/Renilla LUC) (mean±SD; n=3). (C) HCT116 XIAP−/− cells (KO) and XIAP−/− cells (WT) were transfected in 96-well plates with 100 ng of Myc-NOD1 or -NOD2 per well along with 1 ng per well of either empty plasmid or FLAG-XIAP-encoding plasmid. NF-κB activity was measured 24 h after transfection by the Dual luciferase assay (mean±std dev; n=3). (D) HEK293T cells stably over-expressing NOD1 or NOD2 with stably integrated NF-κB-luciferase reporter gene were transduced with control scrambled or XIAP shRNA lentiviruses (multiplicity of infection, MOI>100). Luciferase activity was measurement 12-14 h later, expressing data as mean±std dev of greater than or equal to three replicate determinations performed in at least two independent experiments. (E and F) HEK293T cells were seeded and transfected with plasmids encoding pcDNA Myc-epitope tagged NOD1 (E), NOD2 (F), XIAP shRNA, and/or a control vector together with NF-κB-luciferase reporter gene and Renilla luciferase plasmid for normalization of data, NF-κB activity was measured 24 h posttransfection, and expressed as fold induction relative to cells transfected with control plasmid (mean±SD; n=3) and are representative of three independent experiments. (G and H) HEK293T cells stably expressing an XIAP shRNA were seeded and transfected with plasmids encoding pcDNA Myc-epitope tagged NOD1 (G), or NOD2 (H), or a control vector together with a NF-κB-luciferase report gene. NF-κB activity was measured 24 h later, reporting data as fold activity induction (mean std dev; n=3) (G, Right) Immunoblot analysis was performed on HEK293T stable transfectants for XIAP expression. Lysates were normalized for protein content (20 μg) and blots were probed with antibodies recognizing XIAP and β-actin.

FIGS. 24A-24C show that XIAP binds RIP2. (A) HEK293T cells were co-transfected with plasmids encoding FLAG-XIAP, GFP-RIP2WT, GFP-RIP2ΔCARD, GFP-RIP2Δkinase domain (KD) or empty pEGFP-C2, as indicated. After 24 h, cell lysates were prepared, normalized for protein content, and GFP-tagged proteins were immunoprecipitated using anti-GFP antibody. Immunoprecipitates were analyzed by immunoblotting using antibodies specific for FLAG epitope (Top) or GFP (Middle). Alternatively, cell lysates were analyzed directly by SDS/PAGE/immunoblotting (Bottom). Molecular weight (MW) markers are indicated in kilo-Daltons (kDa). (*HC and *LC indicate Ig heavy and light chains). (B) Lysates of THP-1 cells were immunoprecipitated with control IgG or rat anti-RIP2 antibody. The resulting immunoprecipitates were analyzed by immunoblotting using mouse monoclonal anti-XIAP antibody (Top). The cell lysate (50 μg protein) was also analyzed by SDS/PAGE/immunoblotting using mouse-monoclonal anti-XIAP or rat monoclonal anti-RIP2 (Bottom). (C) Lysates of transfected HEK293T cells expressing FLAG-RIP2 were incubated with recombinant GST-XIAP, various GST-XIAP fragments, or GST-Survivin immobilized on glutathione Sepharose and bound proteins were analyzed by SDS/PAGE/immunoblotting using mouse monoclonal anti-FLAG (Top) and anti-GST (Bottom) antibodies. Asterisks denote nonspecific bands.

FIGS. 25A-25E show that SMAC binding site of BIR2 domain of XIAP is required for RIP2 binding. (A) Schematic representation of GFP-XIAP mutants. (B) Transfected HEK293T cells expressing FLAG-RIP2 together with GFP-XIAPWT, GFPXIAPE219R, GFP-XIAPH223V, GFP-XIAPE219R/H223V or GFP-control were lysed and subjected to immunoprecipitation using anti-FLAG antibody. Immunoprecipitates were analyzed by SDS/PAGE/immunoblotting using anti-FLAG and anti-GFP antibodies. Protein binding was quantified by densitometry analysis, measuring the integrated density value expressed as arbitrary units of the GFP-XIAP bands. Values are expressed as mean±SD of three independent experiments. (C-E) Lysates (1 mg) of transfected HEK293T cells expressing FLAG-RIP2 were incubated with 2 μg of recombinant GST-XIAP immobilized on glutathione-Sepharose along with various amounts of His-6-SMAC protein C, SMAC peptide (D), or SMAC-mimicking compounds ABT-10, nonSMAC-mimicking compound TPI-1396-11, or vehicle control (F). Beads were analyzed by immunoblotting using anti-FLAG-HRP, anti-XIAP/anti-GST or anti-SMAC antibodies as indicated. An aliquot of lysates was also directly analyzed by immunoblotting (“input”).

FIGS. 26A and 26B show that XIAP protein associates with the NOD/RIP2 complex. Myc-NOD1 (A) or Myc-NOD2 (B) were expressed in HEK293T cells along with GFP-RIP2 (wild-type [WT]), GFP-RIP2ΔCARD or GFP-RIP2Δkinase domain (KD). Protein lysates (1 mg) were incubated with GST-XIAP immobilized on glutathione-Sepharose and adsorbed proteins were analyzed by immunoblotting using anti-Myc and anti-GFP antibodies. An aliquot of lysates (input) was analyzed directly by immunoblotting.

### DETAILED DESCRIPTION OF THE INVENTION

The disclosed methods and compositions may be understood more readily by reference to the following detailed description of particular embodiments and the Examples included therein and to the Figures and their previous and following description.

### Definitions

As used herein and in the appended claims, the singular forms “a”, “an”, and “the” include plural reference unless the context clearly dictates otherwise. Thus, for example, reference to “a compound” includes a plurality of such compounds, reference to “the compound” is a reference to one or more compounds and equivalents thereof known to those skilled in the art, and so forth.

“Optional” or optionally means that the subsequently described event, circumstance, or material may or may not occur or be present, and that the description includes instances where the event, circumstance, or material occurs or is present and instances where it does not occur or is not present.

Ranges or values disclosed here may be expressed herein as from “about” one particular value, and/or to “about” another particular value. Unless the context provides otherwise, about refers to ±10, ±5, or ±1 percent of the value.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Activities” of a protein include, for example, transcription, translation, intracellular translocation, secretion, phosphorylation by kinases, cleavage by proteases, homophilic and heterophilic binding to other proteins.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the reduction can be a 10, 20, 30, 40, 50, 60, 70, 80, 90, 100%, or any amount of increase in between as compared to native or control levels.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” means that the amount of the composition used is of sufficient quantity to ameliorate one or more causes or symptoms of a disease or disorder. Such amelioration only requires a reduction or alteration, not necessarily elimination.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

“Pharmaceutically acceptable” refers to non toxic substances suitable for administration to a patient according to the methods provided herein.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

The term “alkyl group” as used herein is a monovalent, branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. In some embodiment, the alkyl group contains 1 to 12 carbon atoms, such as methyl, ethyl, n-propyl, i-propyl, n-butyl, i-butyl, t-butyl, pentyl, hexyl, heptyl, n-octyl, dodecyl, amyl, 2-ethylhexyl, and the like. A “lower alkyl” group is an alkyl group containing from one to six carbon atoms.

The term “alkoxy” as used herein is an alkyl group bound through a single, terminal ether linkage; that is, an “alkoxy” group may be defined as —OR where R is alkyl as defined above. A “lower alkoxy” group is an alkoxy group containing from one to six carbon atoms. An example of alkoxy is the methoxy group CH₃O—.

The term “alkenyl group” as used herein is a hydrocarbon group of from 2 to 24 carbon atoms and structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C.

The term “alkynyl group” as used herein is a hydrocarbon group of 2 to 24 carbon atoms and a structural formula containing at least one carbon-carbon triple bond.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “heteroaryl group” as used herein is an aryl group containing 1-4 ring heteroatoms.

The term “cycloalkyl group” as used herein is a monovalent, mono-, bi-, or tricyclic, non-aromatic carbon-based ring composed of at least three carbon atoms that is fully saturated or partially unsaturated. Examples of cycloalkyl groups include, but are not limited to, cyclopropyl, cyclobutyl, cyclopentyl, cyclohexyl, cyclopentenyl etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “heterocycloalkyl group” as used herein is a cycloalkyl group containing 1-4 ring heteroatoms.

“Cycloalkoxy” refers to a group cycloalkyl-O—. An example of cycloalkoxy is the cyclopropyloxy.

A group “substituted with fluoro” refers to one or more H atoms in that group being replaced with fluorine atoms. An example of an alkyl group substituted with fluoro includes, without limitation, trifluoromethyl.

“Halo” and “halogen” refer to fluoro, chloro, bromo and/or iodo groups.

As used herein, the term “non-natural amino acid” refers to an organic compound that has a structure similar to a natural amino acid so that it mimics the structure and reactivity of a natural amino acid. The non-natural amino acid as defined herein generally increases or enhances the properties of a peptide (e.g., selectivity, stability) when the non-natural amino acid is either substituted for a natural amino acid or incorporated into a peptide.

As used herein, the term “activity” refers to a biological activity, unless the context clearly indicates otherwise.

As used herein, the term “pharmacological activity” refers to the inherent physical properties of a peptide or polypeptide. These properties include but are not limited to half-life, solubility, and stability and other pharmacokinetic properties.

The term “modified” is often used herein to describe polymers and means that a particular monomeric unit that would typically make up the pure polymer has been replaced by another monomeric unit that shares a common polymerization capacity with the replaced monomeric unit. Thus, for example, it is possible to substitute diol residues for glycol in poly(ethylene glycol), in which case the poly(ethylene glycol) will be “modified” with the diol. If the poly(ethylene glycol) is modified with a mole percentage of the diol, then such a mole percentage is based upon the total number of moles of glycol that would be present in the pure polymer but for the modification. Thus, in a poly(ethylene glycol) that has been modified by 50 mole % with a diol, the diol and glycol residues are present in equimolar amounts.

### NOD1, NOD2 and Their Modulation

The modulation of immune response activity is one of the major goals in the development of novel therapeutics for human immune or inflammatory diseases. The innate system resides at the intersection of the pathways of microbial recognition, inflammation, and cell death, thereby offering various therapeutic targets (Ulevitch, R. J. Nat Rev Immunol, 4: 512-520, 2004). In this context. NOD1 and NOD2 are of particular interest, since they recognize distinct structures derived from bacterial peptidoglycans and directly activate NF-κB pathway, which controls the production of pro-inflammatory molecules. Access to chemical inhibitors of NODs will empower research on defining the roles of these proteins in numerous acute and chronic inflammatory diseases, as well as in normal host-defense mechanisms.

The two main molecular targets of interest as described elsewhere herein are the central NACHT domains and the Leucine-Rich Repeat (LLR) domains, which are predicted to be “druggable”. Furthermore, the use of specific inhibitors towards NOD1 and NOD2 proteins can decipher the differential recognition process of peptidoglycans by human cells and consequent signaling pathways. Besides, compounds that inhibit NOD-dependent NF-κB activation will certainly be useful to elucidate alternative ways to find novel cell targets that might counterbalance opposite events like apoptosis and cell survival. These events are typically affected by constitutive NF-κB activation in a variety of pathological conditions, like inflammatory diseases and cancer. Changes on the pattern of post-translational modification (for instance, ubiquitination) and protein binding of partners (XIAP, RICK/RIP2) of NOD complex might be eventually involved on the process of NOD-dependent NF-κB inhibition.

The disclosed methods can be used to identify, indicate, produce, and use, modulators of NOD1, NOD2, or both. A modulator of NOD1, NOD2, or both is a compound, molecule, composition, etc. that affects the expression and/or activity of NOD1, NOD2, or both. Generally, a modulator of NOD1, NOD2, or both will affect the NOD activation pathway (also referred to as the NOD signaling pathway). Modulators of NOD1 and/or NOD2 can activate, stimulate, induce, increase activity of inhibit, repress, decrease activity of, etc. NOD1 and/or NOD2 expression, NOD1 and/or NOD2 activity, NOD1 and/or NOD2 signaling, and/or the NOD activation pathway. Thus, for example, activators of NOD1 and/or NOD2 can activate, stimulate, induce, increase activity of etc. NOD1 and/or NOD2 expression, NOD1 and/or NOD2 activity, NOD1 and/or NOD2 signaling, and/or the NOD activation pathway, Inhibitors of NOD1 and/or NOD2 can inhibit, repress, decrease activity of, etc. NOD1 and/or NOD2 expression, NOD1 and/or NOD2 activity, NOD1 and/or NOD2 signaling, and/or the NOD activation pathway. Potential modulators of NOD1, NOD2, or both are compounds, molecules, compositions. etc. that affect NF-κB and which may do so via the NOD activation pathway. The disclosed methods can be used to indicate that compounds are potential or actual modulators of NOD1, NOD2, or both.

Examples of modulators of NOD1 and/or NOD2 include, for example, compounds having the structure of Formulas A, I, II, III, and IV as disclosed in any aspect or embodiment herein.

NOD signaling can be affected by modulating components and interactions in the NOD signaling pathway. For example, XIAP is required for NOD signaling. Thus, NOD signaling can be modulated by modulating XIAP levels, activity, and/or interaction with components of the NOD signaling pathway. It has been discovered that XIAP interacts with RIP2 and that this interaction is mediated by the BIR2 domain on XIAP and the kinase domain on RIP2. Thus, for example, compounds that disrupt interaction of XIAP and RIP2 can reduce NOD signaling. For example, peptides comprising the BIR2 domain of XIAP but lacking one or more critical XIAP domains and/or functions could be used. Such peptides could compete with XIAP for binding to RIP2. Similarly, peptides comprising the kinase domain of RIP2 but lacking one or more critical RIP2 domains and/or functions could be used. Such peptides could compete with RIP2 for binding to XIAP. Conversely, compounds that strengthen or mimic the effects of XIAP binding to RIP2 can increase NOD signaling. XIAP is involved in other interactions and other signaling pathways and so blocking or inhibiting one or more of these interactions can increase the availability of XIAP for interaction with RIP2. Useful for this purpose would be interactions and activities of XIAP that are not involved in NOD signaling. For example, compounds that compete with binding of XIAP to components via XIAP domains that are not involved in NOD signaling can be used. This could be accomplished, for example, by inhibiting interaction of the BIR3 domain of XIAP with other components. NOD signaling can also be affected by modulating an interaction or function of XIAP needed for NOD signaling other than the XIAP/RIP2 interaction.

The assay can identify and isolate small compounds that inhibit NF-κB pathway, specifically through NOD1 and NOD2 signaling cascades, using a primary cell-based assay based on NF-κB mediated luciferase reporter read-out. As described elsewhere herein a HEK 293T variant cell line was engineered that stably transduced with luciferase reporter gene and further treated it with NOD1- and NOD2-related inducers (gamma-tri-DAP and muramyl dipeptide, respectively). Blockage of NF-κB activation, by the addition of compounds from the NIH library, was monitored as a decrease into luminescence signal. Non-cytotoxic hits (positive compounds) with no inhibitory effects after treatment with non-related NF-κB activators (using TNF-alpha as a prototype) would then be considered for secondary assays to possibly nominate NOD-dependent NF-κB inhibitors.

Disclosed are methods of identifying potential modulators of NOD1, NOD2, or both, the method comprising (a) bringing into contact a test compound and a NOD test cell, and (b) detecting the level of expression of the reporter, wherein a level of expression of the reporter above or below a control level of expression of the reporter indicates that the test compound is a potential modulator of NOD1, NOD2, or both. The NOD test cell can be a mammalian cell comprising an NF-κB-responsive reporter construct. The reporter can be expressed under NOD-inducing conditions. The NOD test cell can be exposed to NOD-inducing conditions. The control level of expression of the reporter is the level of expression of the reporter when the NOD test cell is exposed to the NOD-inducing conditions in the absence of any test compound.

The methods can further comprise repeating steps (a) and (b) with the test compound indicated or further indicated as a potential modulator of NOD1, NOD2, or both. The methods can further comprise repeating steps (a) and (b) using a range of concentrations of the test compound to determine concentration-dependent behavior. The methods can further comprise determining cytotoxicity of the test compound indicated or further indicated as a potential modulator of NOD1, NOD2, or both using an ATP content assay. The methods can further comprise assessing the purity of the test compound indicated or further indicated as a potential modulator of NOD1, NOD2, or both using mass spectrometry. The methods can further comprise identifying if the test compound indicated or further indicated as a potential modulator of NOD1, NOD2, or both competes with ATP for binding to NOD1, NOD2, or both.

The methods can further comprise (c) bringing into contact the test compound, a NOD inducer, and an IL-8 test cell, and (d) detecting the level of Interleukin-8 (IL-8) produced by the IL-8 test cell, wherein a level of IL-8 above or below a control level of IL-8 further indicates that the test compound is a potential modulator of NOD1, NOD2, or both. The IL-8 test cell can be a second mammalian cell comprising a NOD expression construct. NOD1, NOD2, or both can be expressed from the NOD expression construct. The control level of IL-8 can be the level of IL-8 when the IL-8 test cell is exposed to the NOD inducer under the same conditions but in the absence of any test compound. In some forms, NOD1 can be expressed from the NOD expression construct. The NOD inducer can be Ala-γ Glu-diaminopimelic acid (γ-tri-DAP). In some forms, NOD2 can be expressed from the NOD expression construct. The NOD inducer can be muramyldipeptide (MDP). The second mammalian cell can be human breast cancer epithelial MCF-7 cell.

The methods can further comprise testing the test compound indicated or further indicated as a potential modulator of NOD1, NOD2, or both for luciferase inhibition. The methods can further comprise testing the test compound indicated or further indicated as a potential modulator of NOD1, NOD2, or both for modulation of one or more NF-κB activation pathways other than the NOD activation pathway. The methods can further comprise testing the test compound indicated or further indicated as a potential modulator of NOD1, NOD2, or both for modulation of NF-κB activation in the presence of TNF-α, doxorubin, PMA, ionomycin, or a combination. The methods can further comprise identifying if the test compound indicated or further indicated as a potential modulator of NOD1, NOD2, or both are NOD1-specific or NOD2-specific modulators.

The methods can further comprise testing the test compound indicated or further indicated as a potential modulator of NOD1, NOD2, or both for modulation of XIAP. The test compound can be tested for modulation of XIAP by testing the test compound for affecting the interaction of XIAP with RIP2.

The NOD-inducing conditions can comprise the presence of a NOD inducer. The NOD inducer can be Ala-γ Glu-diaminopimelic acid (γ-tri-DAP) or muramyldipeptide (MDP). The NOD-inducing conditions can comprise overexpression of NOD1, NOD2, or both in the NOD test cell. The NOD test cell can comprise a NOD expression construct. The mammalian cell can be a human embryonic kidney (HEK) 293 or 293T cell. The reporter construct can comprise expression control elements. The expression control elements can comprise five tandem HIV NF-κB-responsive elements. The reporter can be firefly luciferase. The reporter construct can be a lentiviral vector.

In some forms, steps (a) and (b) can be performed a plurality of times, wherein a different test compound is used in two or more of the plurality of times steps and (b) are performed. In some forms, a different test compound can be used in each of the plurality of times steps (a) and (b) are performed. In some forms, steps (a) and (b) can be performed the plurality of times simultaneously. In some forms, steps (a) and (b) can be performed the plurality of times in the same device. In some forms, steps (a) and (b) can be performed the plurality of times in a single run. In some forms, steps (a) and (b) can be performed at least 10, 20, 30, 40, 50, 60, 70, 80, 90, 100, 200, 300, 400, 500, 600, 700, 800, 900, or 1000 times.

In some forms, a negative control can be used. The negative control can be the NOD test cell lacking NOD1, NOD2, or both. The negative control can be the NOD test cell in the presence of an IKK inhibitor, an Hsp90 inhibitor, or both.

In some forms, a low or undetectable cytotoxicity can further indicate that the test compound is a potential modulator of NOD1, NOD2, or both. In some forms, a CC₅₀ of greater than 20 μM can further indicate that the test compound is a potential modulator of NOD1, NOD2, or both. In some forms, a concentration-dependent behavior of IC₅₀ of less than 10 μM for NOD1, NOD2, or both can further indicate that the test compound is a potential modulator of NOD1, NOD2, or both. In some forms, a concentration-dependent behavior of IC₅₀ of less than 10 μM for NOD1 with at least a 10-fold selectivity over NOD2 can further indicate that the test compound is a potential modulator of NOD1. In some forms, a concentration-dependent behavior of IC₅₀ of less than 10 μM for NOD2 with at least a 10-fold selectivity over NOD1 can further indicate that the test compound is a potential modulator of NOD2. In some forms, a concentration-dependent behavior of IC₅₀ of less than 10 μM for both NOD1 and NOD2 can further indicate that the test compound is a potential modulator of both NOD1 and NOD2.

In some forms, a lack of effect on the one or more NF-κB activation pathways other than the NOD activation pathway can further indicate that the test compound is a potential modulator of NOD1, NOD2, or both. The NF-κB activation pathway other than the NOD activation pathway can be the TNF-α activation pathway. The NF-κB activation pathway other than the NOD activation pathway can be the doxorubin activation pathway. The NF-κB activation pathway other than the NOD activation pathway can be the PMA activation pathway.

In some forms, a lack of effect on the TNF-α activation pathway can further indicate that the test compound can be a potential modulator of NOD1, NOD2, or both. In some forms, a lack of effect on the doxorubin activation pathway can further indicate that the test compound is a potential modulator of NOD1, NOD2, or both. In some forms, a lack of effect on the PMA activation pathway can further indicate that the test compound is a potential modulator of NOD1, NOD2, or both. In some forms, a ratio of greater than 5 of the IC₅₀ of the one or more NF-κB activation pathways other than the NOD activation pathway to the IC₅₀ of the NOD activation pathway can further indicate that the test compound is a potential modulator of NOD1, NOD2, or both. In some forms, modulation of NF-κB activation can be tested in the presence of γ-tri-DAP and TNF-α, doxorubin, PMA, ionomycin, or a combination.

In some forms, an IC₅₀ of less than 10 μM for NOD1, NOD2, or both can further indicate that the test compound is a potential modulator of NOD1, NOD2, or both. In some forms, an IC₅₀ of less than 10 μM for NOD1 with at least a 10-fold selectivity over NOD2 can further indicate that the test compound is a potential modulator of NOD1. In some forms, an IC₅₀ of less than 10 μM for NOD2 with at least a 10-fold selectivity over NOD1 can further indicate that the test compound is a potential modulator of NOD2. In some forms, an IC₅₀ of less than 10 μM for both NOD1 and NOD2 can further indicate that the test compound is a potential modulator of both NOD1 and NOD2. The test compound indicated or further indicated as a potential modulator of NOD1, NOD2, or both can be an inhibitor, of NOD1, NOD2, or both.

The disclosed methods can include a variety of additional tests and assays to assess compounds identified as potential modulators of NOD1, NOD2, or both. Combinations of such tests and assays can provide a robust selection and winnowing of compounds and the identification of modulators of NOD1, NOD2, or both.

i. IL-8 Assays

Compounds can also, or can further, be screened for an effect on IL-8 levels. For example, the method can comprise bringing into contact the test compound, a NOD inducer, and an IL-8 test cell, wherein the IL-8 lest cell is a second mammalian cell comprising a NOD expression construct, wherein NOD1, NOD2, or both is expressed from the NOD expression construct, and detecting the level of Interleukin-8 (IL-8) produced by the IL-8 test cell, wherein a level of IL-8 above or below a control level of IL-8 further indicates that the test compound is a potential modulator of NOD1, NOD2, or both, wherein the control level of IL-8 is the level of IL-8 when the IL-8 test cell is exposed to the NOD inducer under the same conditions but in the absence of any test compound.

In some forms of the IL-8 assays, NOD1 can be expressed from the NOD expression construct. In some forms, the NOD inducer can be Ala-γ Glu-diaminopimelic acid (γ-tri-DAP). In some forms, NOD2 can be expressed from the NOD expression construct. In some forms, the NOD inducer can be muramyldipeptide (MDP). In some forms, the second mammalian cell can be human breast cancer epithelial MCF-7 cell.

The disclosed screening assays can be aided by use of negative controls and negative control assays. For example, a negative control can be used. The negative control can be, for example, the NOD test cell lacking NOD1, NOD2, or both. The negative control can be, for example, the NOD test cell in the presence of an IKK inhibitor, an Hsp90 inhibitor, or both.

The disclosed screening assays can be aided by, for example, repeating the NOD1/NOD2 assays. For example, steps (a) and (b) can be repeated with the test compound indicated or further indicated as a potential modulator of NOD1, NOD2, or both.

The disclosed screening assays can be aided by, for example, testing compounds for cytotoxicity. For example, the method can comprise determining cytotoxicity of the test compound indicated or further indicated as a potential modulator of NOD1, NOD2, or both using an ATP content assay. In some forms, a low or undetectable cytotoxicity can further indicate that the test compound is a potential modulator of NOD1, NOD2, or both. In some forms, an IC₅₀ of greater than 20 μM can further indicates that the test compound is a potential modulator of NOD1, NOD2, or both (and can indicate that the compound is not prohibitively cytotoxic).

The disclosed screening assays can be aided by, for example, testing compounds for direct inhibition of the reporter (for example, luciferase). Inhibition of the reporter could indicate that the screening assay results are due to an effect on the reporter. For example, the method can comprise testing the test compound indicated or further indicated as a potential modulator of NOD1, NOD2, or both for luciferase inhibition (and can indicate that the compound is not directly affecting the reporter).

The disclosed screening assays can be aided by, for example, testing the purity of the compound. This can be useful to eliminate the possibility that contaminants are causing effects. For example, the method can comprise assessing the purity of the test compound indicated or further indicated as a potential modulator of NOD1, NOD2, or both using mass spectrometry.

ii. Potency and Specificity Assays

The disclosed screening assays can be aided by, for example, determining the potency of compounds and/or determining the specificity of compounds. This can be useful for identifying compounds with sufficient activity to be useful and to identify selective modulators. Selective modulators can allow dissection of activation and signaling pathways. For example, the method can comprise repeating steps (a) and (b) using a range of concentrations of the test compound to determine concentration-dependent behavior.

In some forms, a concentration-dependent behavior of IC₅₀ of less than 1, 2, 3, 4, 5, 6, 7, 8, 9, 10, 11, 12, 13, 14, 15, 16, 17, 18, 19, 20, 25, or 30 μM for NOD1, NOD2, or both can further indicate that the test compound is a potential modulator of NOD1, NOD2, or both. In some forms, a concentration-dependent behavior of IC₅₀ of less than 1, 2, 3, 4, 5, 6, 7, 8, 9, 10, 11, 12, 13, 14, 15, 16, 17, 18, 19, 20, 25, or 30 μM for NOD1 with at least a 1-, 2-, 3-, 4-, 5-, 6-, 7-, 8-, 9-, or 10-fold selectivity over NOD2 can further indicate that the test compound is a potential modulator of NOD1. In some forms, a concentration-dependent behavior of IC₅₀ of less than 1, 2, 3, 4, 5, 6, 7, 8, 9, 10, 11, 12, 13, 14, 15, 16, 17, 18, 19, 20, 25, or 30 μM for NOD2 with at least a 1-, 2-, 3-, 4-, 5-, 6-, 7-, 8-, 9-, or 10-fold selectivity over NOD1 can further indicates that the test compound is a potential modulator of NOD2. In some forms, a concentration-dependent behavior of IC₅₀ of less than 1, 2, 3, 4, 5, 6, 7, 8, 9, 10, 11, 12, 13, 14, 15, 16, 17, 18, 19, 20, 25, or 30 μM for both NODE and NOD2 can further indicates that the test compound is a potential modulator of both NOD1 and NOD2. A concentration-dependent behavior refers to an effect or behavior that changes based on the concentration of the compound. Useful concentration-dependent behaviors include, for example, activation, inhibition, effectiveness, etc.

iii. NF-κB Selectivity Screens

The disclosed screening assays can be aided by, for example, selective effect on the NOD activation pathway (or on other activation or signaling pathways). For example, the method can comprise testing the test compound indicated or further indicated as a potential modulator of NOD1, NOD2, or both for modulation of one or more NF-κB activation pathways other than the NOD activation pathway. In some forms, a lack of effect on the one or more NF-κB activation pathways other than the NOD activation pathway can further indicates that the test compound is a potential modulator of NOD1, NOD2, or both (and can indicate that the compound is selective for the NOD activation pathway).

In some forms, the NF-κB activation pathway other than the NOD activation pathway can be the TNF-α activation pathway. In some forms, a lack of effect on the TNF-α activation pathway can further indicate that the test compound is a potential modulator of NOD1, NOD2, or both (and can indicate that the compound is selective for the NOD activation pathway).

In some forms, the NF-κB activation pathway other than the NOD activation pathway can be the doxorubin activation pathway. In some forms, a lack of effect on the doxorubin activation pathway can further indicate that the test compound is a potential modulator of NOD1, NOD2, or both (and can indicate that the compound is selective for the NOD activation pathway).

In some forms, the NF-κB activation pathway other than the NOD activation pathway can be the PMA activation pathway. In some forms, a lack of effect on the PMA activation pathway can further indicate that the test compound is a potential modulator of NOD1, NOD2, or both (and can indicate that the compound is selective for the NOD activation pathway).

In some forms, a ratio of greater than 2, 3, 4, 5, 6, 7, 8, 9, or 10 of the IC₅₀ of the one or more NF-κB activation pathways other than the NOD activation pathway to the IC₅₀ of the NOD activation pathway can further indicate that the test compound is a potential modulator of NOD1, NOD2, or both. In some forms, the method can comprise testing the test compound indicated or further indicated as a potential modulator of NOD1, NOD2, or both for modulation of NF-κB activation in the presence of TNF-α, doxorubin, PMA, ionomycin, or a combination. In some forms, modulation of NF-κB activation can be tested in the presence of γ-tri-DAP and TNF-α, doxorubin, PMA, ionomycin, or a combination.

The disclosed screening assays can be aided by, for example, identifying if the test compound indicated or further indicated as a potential modulator of NOD1, NOD2, or both competes with ATP for binding to NOD1, NOD2, or both.

The disclosed screening assays can be aided by, for example, identifying if a compound is a specific modulator for NOD1 or NOD2. For example, the method can comprise identifying if the test compound indicated or further indicated as a potential modulator of NOD1, NOD2, or both are NOD1-specific or NOD2-specific modulators. In some forms, an IC₅₀ of less than 1, 2, 3, 4, 5, 6, 7, 8, 9, 10, 11, 12, 14, 15, 16, 17, 18, 19, 20, 25, or 30 μM for NOD1, NOD2, or both can further indicate that the test compound is a potential modulator of NOD1, NOD2, or both (and can indicate that the compound is usefully potent).

In some forms, an IC₅₀ of less than 1, 2, 3, 4, 5, 6, 7, 8, 9, 10, 11, 12, 13, 14, 15, 16, 17, 18, 19, 20, 25, or 30 μM for NOD1 with at least a 1-, 2-, 3-, 4-, 6-, 7-, 8-, 9-, or 10-fold selectivity over NOD2 can further indicate that the test compound is a potential modulator of NOD1 (and can indicate that the compound is selective for NOD1). In some forms, an IC₁₋₅₀ of less than 1, 2, 3, 4, 5, 6, 7, 8, 9, 10, 11, 12, 13, 14, 15, 16, 17, 18, 19, 20, 25, or 30 μM for NOD2 with at least a 1-, 2-, 3-, 4-, 5-, 6-, 7-, 8-, 9-, or 10-fold selectivity over NOD1 can further indicate that the test compound is a potential modulator of NOD2 (and can indicate that the compound is selective for NOD2). In some forms, an IC₅₀ of less than 1, 2, 3, 4, 5, 6, 7, 8, 9, 10, 11, 12, 13, 14, 15, 16, 17, 18, 19, 20, 25, or 30 μM for both NOD1 and NOD2 further indicates that the test compound is a potential modulator of both NOD1 and NOD2 (and can indicate that the compound is not selective for NOD1 or NOD2). In some forms, it can be determined that the test compound indicated or further indicated as a potential modulator of NOD1, NOD2, or both is an inhibitor of NOD1, NOD2, or both.

In one embodiment the assay can be a cell-based HTS assay that utilizes an NF-κB-driven luciferase reporter gene as a measure of NOD1 and NOD2 activity. The assay can also include a secondary assays to confirm compound selectivity towards NOD activity, by measuring production of interleukin-8 (IL-8), on endogenous NF-κB target gene. When combined with insights provided by cheminformatics analysis, as well as a battery of downstream assays we will provide for hit deconvolution, candidate compounds can be identified and further optimized using medicinal chemistry.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds, Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are available from a number of sources, including Biotics (Sussex, UK), Xenova (Slough, UK), Harbor Branch Oceangraphics Institute (Ft. Pierce, Fla.), and PharmaMar. U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the pathways and diseases disclosed herein.

Also disclosed is a process for making a modulator of NOD-like Receptor (NLR), the method comprising manufacturing the compound identified by the method disclosed herein.

Also disclosed are methods of modulating NOD1, NOD2 or both in a subject, comprising administering to a subject a composition comprising a compound having the structure of Formula A, I, II, III, and IV as disclosed herein in various aspects and embodiments.

Also disclosed are methods of modulating NOD1, NOD2 or both in a subject, comprising administering to a subject a composition comprising a compound that binds to or mimics the BIR2 domain of XIAP.

In some forms, the methods can further comprise, prior to administering the composition, identifying the subject as a subject in need of modulation of NOD1, NOD2, or both. The methods can further comprise, prior to administering the composition, diagnosing the subject with a disease associated with NOD1, NOD2 or both. The methods can further comprise, prior to administering the composition, identifying the subject as a subject in need of modulation of NF-κB activity. The methods can further comprise, prior to administering the composition, identifying the subject as a subject in need of inhibition of NF-κB activity. The methods can further comprise, prior to administering the composition, identifying the subject as a subject in need of modulation of Interferon Response Factor activity, AP-1 activity, INK activity, p38 MAPK activity, XIAP activity, or a combination. The methods can further comprise, prior to administering the composition, identifying the subject as a subject in need of inhibition of Interferon Response Factor activity, AP-1 activity, JNK activity, p38 MAPK activity, XIAP activity, or a combination.

The subject can be in need of modulation of NOD1, NOD2, or both. The subject can have been diagnosed with a disease associated with NOD1, NOD2 or both. The disease can be an inflammatory disease. The disease can be Chrohn's disease, Blau Syndrome, early-onset sarcoidosis or atopic diseases. The subject can be in need of modulation of NF-κB activity. The subject can be in need of inhibition of NF-κB activity. The subject can be in need of modulation of Interferon Response Factor activity, AP-1 activity, JNK activity, p38 MAPK activity, XIAP activity, or a combination. The subject can be in need of inhibition of Interferon Response Factor activity, AP-1 activity, JNK activity, p38 MAPK activity, XIAP activity, or a combination.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Compounds

The disclosed methods can make use of various compounds. The disclosed methods can make use of test compounds. A test compound is any compound, molecule, composition. etc. the activity and/or effect of which can be tested in the disclosed methods. For example, the ability of a test compound to modulate NOD1. NOD2, or both can be tested in the disclosed methods. Any suitable compound, molecule, composition, etc. can be used as a test compound. Particularly useful test compounds are small molecules and peptides. For example, compounds in compound libraries and collections can be used. Numerous such libraries and collections are known and can be used. Novel compounds can also be used in the disclosed methods as test compounds.

In one aspect utilized herein are compounds having the structure of Formula I:

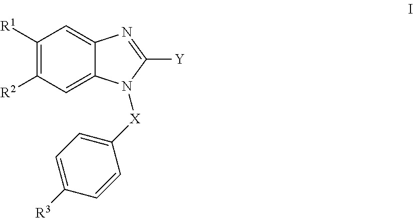

or the pharmaceutically acceptable salt or ester thereof, wherein R¹ and R² are independently hydrogen or C₁-C₃ alkyl; R³ is hydrogen, C₁-C₃ alkyl, C₁-C₃ alkenyl, C₁-C₃ alkoxy, or halogen; X is —(CH₂)₁₋₃—, —(CH₂)₁₋₃O—, —(CH₂)₁₋₂O(CH₂)₁₋₂—, —SO₂—, —(CH₂)₁₋₃SO₂—, —C(O)—, —(CH₂)₁₋₃C(O)—, —(CH₂)₁₋₂C(O)(CH₂)₁₋₂—, or —NH—, —N(R⁴)—; R⁴ is C₁-C₃ alkyl; and Y is hydrogen, amino, C₁-C₃ alkylamino, thio, C₁-C₃ alkylthio, C₁-C₃ alkyl, C₁-C₃ alkoxy, or halogen.

In some forms, R³ can be methylene, methoxy, Cl or F. In some forms, X can be —SO2-. In some forms, Y can be amino or C₁-C₃ alkylamino. In some forms, R¹ and R² can both be hydrogen. In some forms, R¹ and R² can both be hydrogen, Y can be amino, X can be —SO₂—, and R³ can be methylene or Cl.

Also utilized herein are compounds of Formula II:

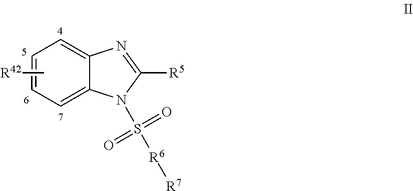

wherein:
one of positions 4, 5, 6 or 7 can optionally be aza substituted;

R⁴² can be hydrogen, C₁-C₃ alkyl, C₁-C₃ alkenyl, C₁-C₃ alkoxy, C₁-C₃ haloalkyl, C₁-C₃ alkylamino, amino, aminoacetyl, nitro, nitrile, halogen, —CO₂R²¹ or —C(O)N(R²²)(R²³);

R²¹ can be hydrogen or C₁-C₃ alkyl;

R²² and R²³ can be independently hydrogen or C₁-C₃ alkyl;

R⁵ can be hydrogen, amino, thin, C₁-C₃ alkylthio, C₁-C₃ alkoxy, hydroxyl, —N(R²³)(R²⁴), C₁-C₃ alkylamino, C₁-C₃ alkylaminoacetyl, or —NH(CH₂)₁₋₃OH;

R²³ and R²⁴ can independently be hydrogen or C₁-C₃ alkyl;

R⁶ can be present or absent, if present R⁶ can be —(CH₂)₁₋₃—;

R⁷ can be aryl, heteroaryl, cycloalkyl or heterocyclyl.

In some forms of the methods, R⁴² can be hydrogen and one of positions 4, 5, 6 or 7 can optionally aza substituted. In one forms, position 4 can be aza substituted. In one forms, position 5 can be aza substituted. In one forms, position 6 can be aza substituted. In one forms, position 7 can be aza substituted.

In some forms, R⁴² can be in position 4. In some forms, R⁴² can be hydrogen, C₁-C₃ alkyl, amino, nitro or aminoacetyl. In some forms R⁴² can be methylene.

In some forms, R⁴² can be in position 5. In some forms, R⁴² can be hydrogen, C₁-C₃ alkyl, C₁-C₃ alkoxy, halogen, C₁-C₃ haloalkyl, nitrite, —CO₂R²¹ or —C(O)N(R²²)(R²³). In some forms R²¹ can be hydrogen or C₁-C₃ alkyl. In some forms, R²¹ can be ethylene. In some forms R²² and R²³ can independently be hydrogen or C₁-C₃ alkyl. In some forms, R²² and R²³ can independently be methylene. In some forms. R⁴² can be —CF₃, Cl, F, nitrile, —CO₂H, —CO₂Et, —CON(Me)₂, methoxy or methylene.

In some forms R⁴² can be in position 6. In some forms, R⁴² can be hydrogen, C₁-C₃ alkyl, C₁-C₁ alkoxy, halogen, C₁-C₃ haloalkyl, nitrite, —CO₂R²¹ or —C(O)N(R²²)(R²³). In some forms, R²¹ can be hydrogen or C₁-C₃ alkyl. In some forms, R²² and R²³ can be independently hydrogen or C₁-C₃ alkyl. In some forms R⁴² can be —CF₃, Cl, F, nitrile, —CO₂H, —CO₂Et, —CON(Me)₂, methoxy or methylene.

In some forms, R⁴² can be in position 7. In some forms, R⁴² can be C₁-C₃ alkyl, amino, nitro or aminoacetyl. In some forms, R⁴² can be methylene.

In some forms, R⁵ can be amino, hydroxyl, —NHMe, N(Me)₂, CH₂NH₂, CH₂NHAc or NHCH₂CH₂OH.

In some forms, R⁶ can be present. In some forms R⁶ can be —CH₂ or —(CH₂)₂—.

In some forms R⁶ can be absent.

In some forms R⁷ can be:

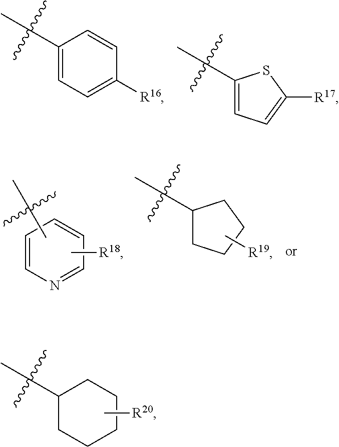

In some forms, R¹⁶ can be hydrogen, C₁-C₃ alkyl, C₁-C₃ alkenyl, C₁-C₃ alkoxy or halogen. In some forms, R¹⁶ can be hydrogen, methylene, Cl or F. In some forms, R¹⁷ can be hydrogen, C₁-C₃ alkyl, C₁-C₃ alkenyl, C₁-C₃ alkoxy or halogen. In some forms. R¹⁷ can be hydrogen or halogen. In some forms, R¹⁷ can be hydrogen, Cl or F. In some forms, R¹⁸ can be hydrogen, C₁-C₃ alkyl, C₁-C₃ alkenyl, C₁-C₃ alkoxy or halogen. In some Corms R¹⁸, can be hydrogen. In some forms. R¹⁹ can be hydrogen, C₁-C₃ alkyl, C₁-C₃ alkenyl, C₁-C₃ alkoxy or halogen. In some forms, R¹⁹ can be hydrogen. In some forms, R²⁰ can be hydrogen, C₁-C₃ alkyl, C₁-C₃ alkenyl, C₁-C₃ alkoxy or halogen. In some forms R²⁰ can be hydrogen.

Also utilized herein are compounds of Formula III.

In certain embodiments, the compounds of Formula III utilized herein are those tabulated below:

TABLE 1

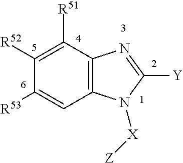

| | | | | | | | | Inhibition |
|---|---|---|---|---|---|---|---|---|
| | | | | | | | | of IL-8 |
| Cmpd | | | | | (NOD1) | (NOD2) | TNF-α | secretion |
| # | R⁵¹, R⁵², R⁵³ | Z | X | Y | IC₅₀(μM) | IC₅₀(μM) | IC₅₀(μM) | IC₅₀(μM) |
| 1 | R⁵¹ = R⁵² = R⁵³ = H | 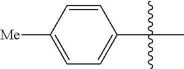 | SO₂ | NH₂ | 0.56 ± 0.04 | >20 | >20 | 0.62 |
| 2 | R⁵¹ = R⁵² = R⁵³ = H | 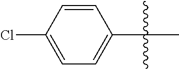 | SO₂ | NH₂ | 0.09 ± 0.01 | >20 | >20 | 1.46 |
| 3 | R⁵¹ = R⁵² = R⁵³ = H | 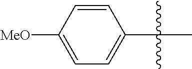 | SO₂ | NH₂ | 2.7 ± 0.69 | >20 | >20 | 3.13 |
| 4 | R⁵¹ = R⁵² = R⁵³ = H | 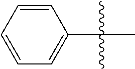 | SO₂ | NH₂ | 2.2 ± 0.21 | >20 | >20 | 2.41 |
| 5 | R⁵¹ = R⁵² = R⁵³ = H | 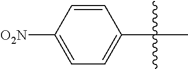 | SO₂ | NH₂ | 14 ± 1.8 | >20 | >20 | >5 |
| 6 | R⁵¹ = R⁵² = R⁵³ = H | 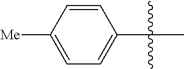 | SO₂ | H | 6.3 ± 0.81 | >20 | >20 | >5 |
| 7 | R⁵¹ = R⁵² = R⁵³ = H | 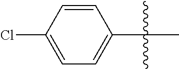 | CH₂ | NH₂ | 7.7 ± 0.82 | 11.9 ± 0.5 | >20 | |
| 8 | R⁵¹ = R⁵² = R⁵³ = H | 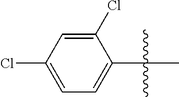 | CO | NH₂ | 2.8 ± 0.57 | 3.8 ± 1.5 | 3.2 | |
| 9 | R⁵¹ = R⁵² = R⁵³ = H | 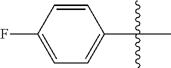 | CO | NH₂ | 18 ± 2.0 | >20 | >20 | |
| 10 | R⁵¹ = R⁵² = R⁵³ = H | 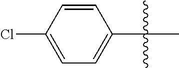 | CH₂CH₂CO | NH₂ | 16.3 ± 3.7 | >20 | >20 | |
| 11 | R⁵¹ = R⁵² = R⁵³ = H | 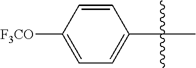 | SO₂ | NH₂ | 8.94 ± 0.57 | >20 | >20 | |
| 12 | R⁵¹ = R⁵² = R⁵³ = H | 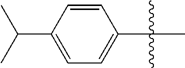 | SO₂ | NH₂ | 0.50 ± 0.11 | >20 | >20 | |
| 13 | R⁵¹ = R⁵² = R⁵³ = H | 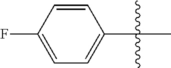 | SO₂ | NH₂ | 4.31 ± 0.23 | >20 | >20 | |
| 14 | R⁵¹ = R⁵² = R⁵³ = H | 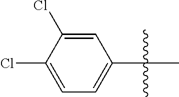 | SO₂ | NH₂ | 0.53 ± 0.07 | >20 | >20 | |
| 15 | R⁵¹ = R⁵² = R⁵³ = H | 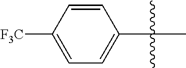 | SO₂ | NH₂ | 0.96 ± 0.06 | >20 | >20 | |
| 16 | R⁵¹ = R⁵² = R⁵³ = H | 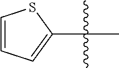 | SO₂ | NH₂ | 11.0 | >25 | 6.6 | |
| 17 | R⁵¹ = R⁵² = R⁵³ = H | 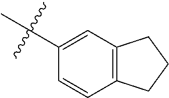 | SO₂ | NH₂ | 0.8 | 0.6 | >25 | |
| 18 | R⁵¹ = R⁵³ = H, R⁵² = Me | 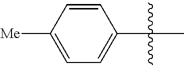 | SO₂ | NH₂ | 9.3 | 15 | >25 | |
| 19 | R₁ = Me, R₂ = R₃ = H | 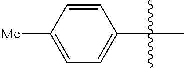 | SO₂ | NH₂ | 2.6 | >25 | >25 | |
| 20 | R⁵¹ = R⁵³ = H, R⁵² = CF₃ | 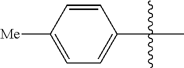 | SO₂ | NH₂ | 1.6 | 4.7 | 2.3 | |
| 21 | R⁵¹ = R⁵² = H, R⁵³ = CF₃ | 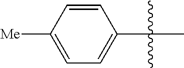 | SO₂ | NH₂ | 3.9 | 10.0 | 12.3 | |
| 22 | R₁ = R₃ = H, R₂ = F | 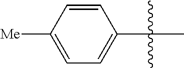 | SO₂ | NH₂ | 1.8 | >25 | >25 | |
| 23 | R⁵¹ = R⁵² = H, R⁵³ = F | 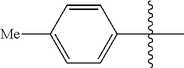 | SO₂ | NH₂ | 6.2 | >25 | >25 | |
| 24 | R⁵¹ = R⁵³ = H, R⁵² = OMe | 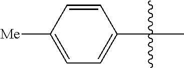 | SO₂ | NH₂ | 1.9 | 3.1 | >25 | |
| 25 | R⁵¹ = R⁵² = H, R⁵³ = COOEt | 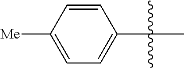 | SO₂ | NH₂ | 3.0 | 7.6 | >25 | |
| 26 | R⁵¹ = —NH₂, R⁵² = R⁵³ = H | 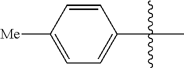 | SO₂ | NH₂ | 17.5 | 12.8 | >25 | |
| 27 | R⁵¹ = R⁵² = R⁵³ = H | 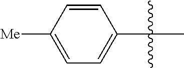 | SO₂ | —CH₂NHCHO | 10.9 | 15.6 | 8.4 | |
| 28 | R⁵¹ = R⁵² = R⁵³ = H | 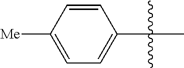 | SO₂ | —NH(CH₂)₃OH | 12.1 | >25 | >25 | |
| 29 | R⁵¹ = R⁵² = R⁵³ = H | 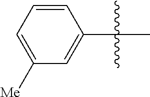 | SO₂ | NH₂ | 3.8 | | >100 | |
| 30 | R⁵¹ = R⁵² = R⁵³ = H | 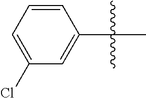 | SO₂ | NH₂ | 3.5 | | >100 | |
| 31 | R⁵¹ = R⁵² = R⁵³ = H | 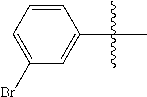 | SO₂ | NH₂ | 3.5 | | >100 | |
| 32 | R⁵¹ = R⁵² = R⁵³ = H | 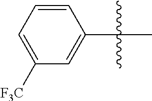 | SO₂ | NH₂ | 12.4 | | >100 | |
| 33 | R⁵¹ = R⁵² = R⁵³ = H | 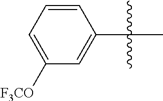 | SO₂ | NH₂ | >25 | | >100 | |
| 34 | R⁵¹ = R⁵² = R⁵³ = H | 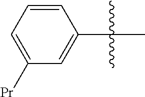 | SO₂ | NH₂ | 1.3 | | >100 | |
| 35 | R⁵¹ = R⁵² = R⁵³ = H | 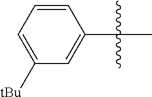 | SO₂ | NH₂ | 3.3 | | 15.6 | |
| 36 | R⁵¹ = R⁵² = R⁵³ = H | 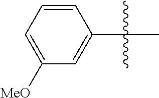 | SO₂ | NH₂ | 14.5 | | >100 | |
| 37 | R⁵¹ = R⁵² = R⁵³ = H | 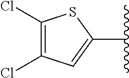 | SO₂ | NH₂ | 2.2 | | 18.4 | |
| 38 | R⁵¹ = R⁵² = R⁵³ = H | 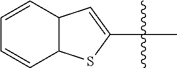 | SO₂ | NH₂ | 0.8 | | >100 | |
| 39 | R⁵¹ = R⁵² = R⁵³ = H | 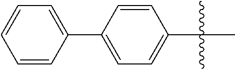 | SO₂ | NH₂ | 1.5 | | 4.2 | |
| 40 | R⁵¹ = R⁵² = R⁵³ = H | 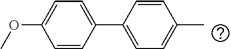 | SO₂ | NH₂ | 12.7 | | 5.2 | |
| 41 | R⁵¹ = R⁵² = R⁵³ = H | 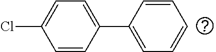 | SO₂ | NH₂ | 1.4 | | 0.97 | |

In one embodiment, the compounds utilized herein are potent (<1 micro molar IC₅₀) and selective inhibitors of NOD1 (over NOD2) induced NF-κB activation. In one embodiment, the compounds utilized herein are about 5-20 fold, about 15 fold, or about 10 fold selective for inhibiting NOD1 over NOD2. In another embodiment, the compound utilized herein is about 2-10 fold, about 5 fold, or about 7 Cold selective for inhibiting NOD1 mediated NF-κB activation compared to tumor necrosis factor-α (TNF-α) mediated NF-κB activation. The compounds utilized are tested for efficacy as described herein in the Examples section and/or following methods well known to the skilled artisan.

In one embodiment, also provided herein are novel compounds that are capable of modulating NOD1 mediated NF-κB activation. In one embodiment, such novel compounds have the structure of Formula IV:

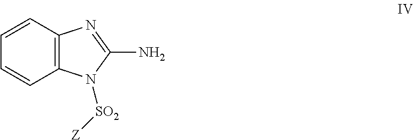

wherein Z is:

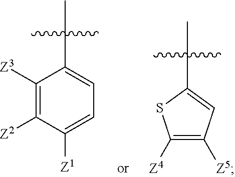

Z¹ is H, C₁-C₆ alkyl, C₁-C₆ alkyl substituted with fluoro, C₁-C₆ alkoxy, or halo; Z² is C₁-C₆ alkyl, C₁-C₆ alkoxy, or halo, or Z₁ and Z₂ together with the carbon atoms they are attached to form a 5 membered ring containing carbon ring atoms; Z³ is H; and Z⁴ and Z⁵ together with the carbon atoms they are attached to form an aromatic ring; or a pharmaceutically acceptable salt thereof.

The compounds represented by Formulas A, I, II, III, and IV can be optically active or racemic. For example, the stereochemistry at one or more carbons in the Formulas above can vary, and will depend upon the spatial relationship between ring groups to one another. In one aspect, the stereochemistry at one or more of the carbons in is S. In another aspect, the stereochemistry at one or more of the carbons is R. Using techniques known in the art, it is possible to vary the stereochemistry at one or more of the carbons.

Also described herein are pharmaceutically acceptable nontoxic ester, amide, and salt derivatives of those compounds, such as compounds of Formulas A, I, II, III, and IV containing a carboxylic acid moiety.

The disclosed compounds, such as compounds of Formulas A, I, II, III, and IV also encompasses pharmaceutically acceptable esters, amides, and salts of such compounds, as described in detail elsewhere herein.

The disclosed compounds, such as compounds of Formulas A, I, II, III, and IV and their pharmaceutically acceptable esters, amides, and salts are referred to herein as the inventive compounds.

The disclosed compounds, such as compounds of Formulas A, I, II, III, and IV also encompass pharmaceutically acceptable salts. Pharmaceutically acceptable salts can be prepared by treating the free acid with an appropriate amount of a pharmaceutically acceptable base. Representative pharmaceutically acceptable bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine. 2-dimethylaminoethanol, 2-diethylaminoethanol, lysine, arginine, histidine, and the like. In some forms, the reaction is conducted in water, alone or in combination with an inert, water-miscible organic solvent, at a temperature of from about 0° C. to about 100° C. such as at room temperature. The molar ratio of the disclosed compounds, such as compounds of Formulas A, I, II, III, and IV to base used are chosen to provide the ratio desired for any particular salts. For preparing, for example, the ammonium salts of the free acid starting material, the starting material can be treated with approximately one equivalent of pharmaceutically acceptable base to yield a neutral salt.

The compounds utilized herein are synthesized following various methods disclosed herein and adapting various known methods in accordance with the disclosure here.

Benzimidazole compounds of formula A were synthesized as shown below.

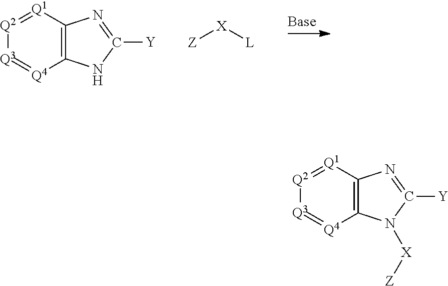

wherein L is a leaving group, nonlimiting examples of which include halo and —OSO₂R^{L} wherein R^{L} is a substituted or unsubstituted alkyl or aryl group.

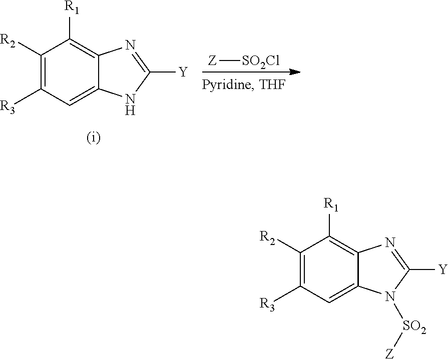

As shown above, benzimidazole compounds (i) were reacted with various sulfonyl chlorides in the presence of pyridine to obtain the N-sulfonylated benzimidazole compounds.

Ester derivatives are typically prepared as precursors to the acid form of the compounds—as illustrated in the examples below—and accordingly can serve as prodrugs. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

The compounds of these inventions were identified using cell-based high through put (FITS) assays with an NO-κB-driven luciferase reporter gene as a measure of NOD1 or NOD2 activity. For the NOD1 assay, HEK293T cells were stimulated with NOD1 ligand, Ala-γ-Gludiaminopimelic acid (γ-tri-DAP), a component of peptidoglycan (PG), relying on endogenous NOD1 expression to result in NF-κB reporter gene activation (PubChem AID 1578). The Z′ values for the optimized assay performed in either 384 or 1536 well format were consistently in the range of 0.67 to 0.73. The NOD2 assay utilized stable over-expression of NOD2 in HEK293T cells, which employed the same NF-κB luciferase reporter gene and which was also optimized to Z′ factor>0.5 in both 384 and 1536 well formats (PubChem AID 1566).

The NTH library (˜300,000 compounds) was screened at an average concentration of ˜4 μM using the NOD1 and NOD2 HTS assay in 1536 well format to identify candidate inhibitors based on NF-κB reporter gene activity. Hits were counter-screened to eliminate cytotoxic compounds (false-positives), and were counterscreened using cheminformatic filters to eliminate historically promiscuous bioactives. Hits that were identified to inhibit either NOD1 and/or NOD2 were then further tested at the same concentration against the same HEK293T-NF-κB luciferase cells stimulated with TNF-α to induce NF-κB by an alternative means (PubChem AID 1852), thus eliminating non-specific compounds. The hit compounds were retested, thereby reducing the number of confirmed hits. Testing these compounds in dose-response experiments using both NOD1 and NOD2 NF-κB reporter gene assays revealed compounds with IC₅₀≦10 μM and with little or no cytotoxicity at 20 μM (PubChem AID 2335). Counter-screening the NOD1 and NOD2 hits against each other revealed compounds showing >10-fold target selectivity of NOD1 over NOD2.

Furthermore, pathway selectivity assays revealed NOD1-selective nature of the inhibitors utilized according to this technology. Several cell-based assays were developed to differentiate compounds that inhibit NF-κB induction by other upstream activators from NOD1/NOD2 selective compounds. For instance, using the same HEK293T-NF-κB-luciferase cells, the ability of compounds to suppress NF-κB activity induced by NOD1 ligand (γ-tri-DAP), NOD2 ligand (muramyl dipeptide [MDP]), TNFα, protein kinase C activators (phorbol myristic acetate [PMA] and ionomycin), and DNA damaging agents (doxorubicin) were compared. Consistently, various benzimidazole derivatives inhibited NF-κB activation only after γ-tri-DAP treatment, thus showing potential NOD1-specific NF-κB inhibitors.

To extend the analysis of the candidate NOD1 inhibitors beyond reporter gene assays, the levels of a NF-κB-inducible cytokine, interleukin-8 (IL-8), was also measured. Using an assay employing breast cancer MCF-7 cells over-expressing NOD1 or NOD2, IL-8 secretion into culture supernatants following stimulation with NOD1 ligand (γ-tri-DAP), NOD2 ligand (MDP), or TNF-α were measured. Again, the active benzimidazole compounds selectively inhibited IL-8 production induced by NOD1 ligand but not by other stimuli. Compound 1 also inhibited γ-tri-DAP-induced expression of the prototypical NF-κB target gene IkBα at the mRNA level.

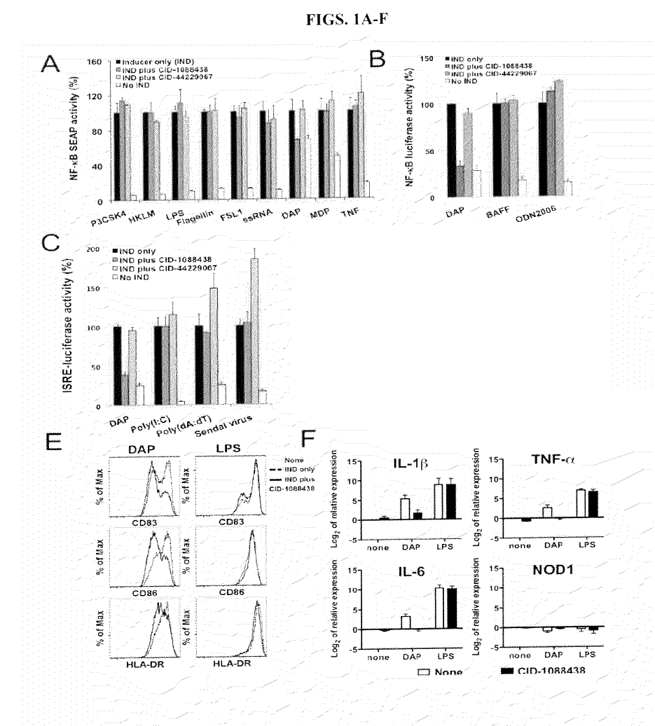

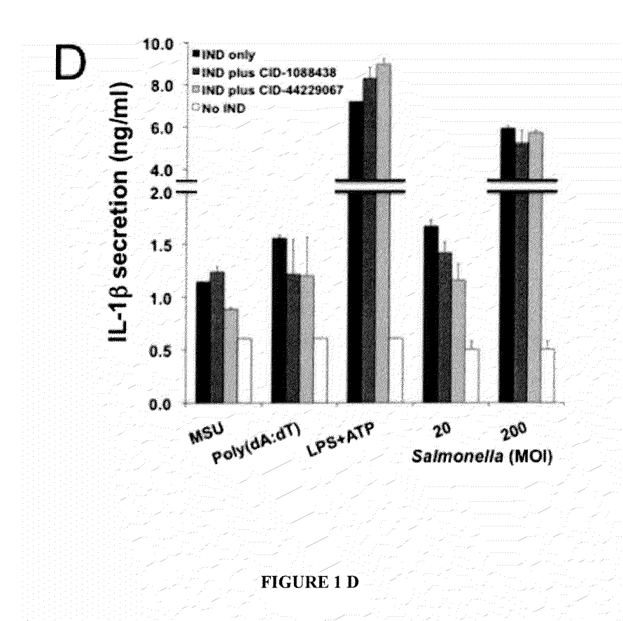

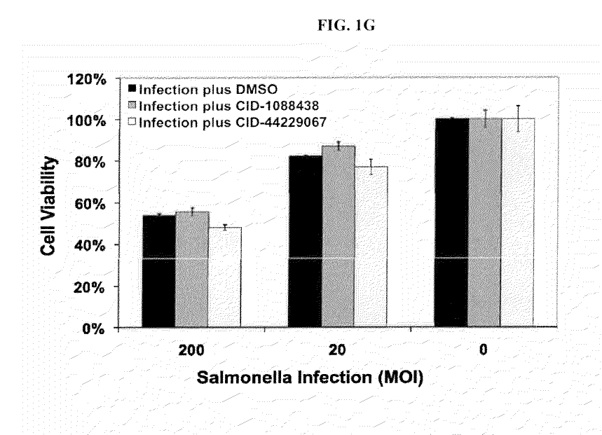

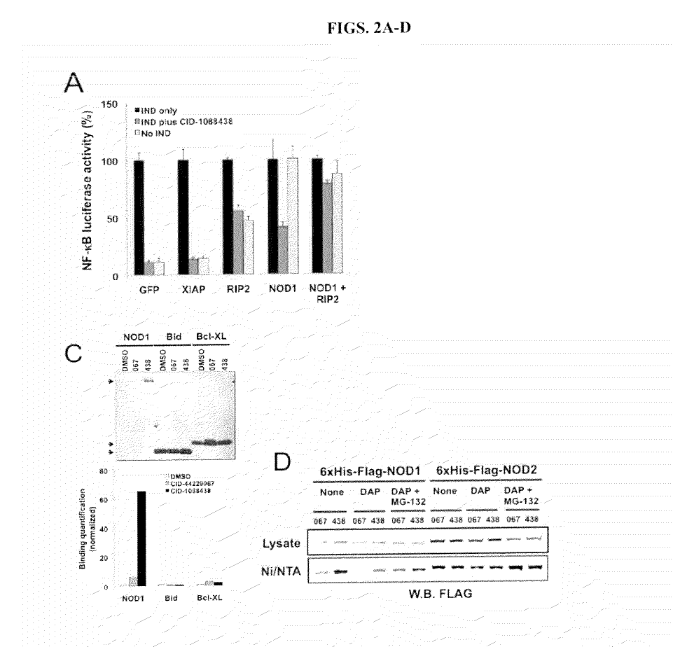

In addition to NLRs, Toll-like receptors (TLRs) and RIG-1-like receptors (RLRs) constitute important families of pathogen receptors (Creagh and O'Neill, 2006). Human myelomonocytic THP-1 cells containing a NF-κB/AP-1-inducible reporter gene encoding secreted alkaline phosphatase (SEAP) were employed for convenient monitoring of NF-κB activity. After inducing differentiation with PMA, THP.1 macrophages were treated for 24 hours with compound 1 or a negative control and various TLR agonists, assessing effects on NF-κB-inducible SEAP activity. No inhibitory effects or compound 1 were observed for any of the TLR agonists tested (TLR1, 2, 4, 5, 6 and 8) (FIG. 1A). While the NOD1 ligand γ-tri-DAP is a weak inducer of NF-κB activity in THP.1 macrophages, inhibition by compound 1 was highly reproducible.

Using 697 pre-B leukemia cells containing a NF-κB-luciferase reporter gene, it was verified that the noncanonical NF-κB activation induced by BAFF is not inhibited by CID-1088438 (FIG. 1B), whereas NF-κB activity induced by NOD1 ligand γ-tri-DAP is inhibited. Compound 1 also did not inhibit NF-κB activity induced by TLR9 ligand CpG DNA in these cells (FIG. 1B). The RIG-I like receptors (RLRs) comprise a family of cytoplasmic RNA helicases that include RIG-I (retinoic-acid-inducible protein I), and MDA-5 (melanoma differentiation-associated gene 5), implicated in viral double-strand RNA recognition. RIG-I and MDA-5 bind the mitochondrial membrane protein MAVS to initiate a signaling cascade that includes induction of the type I interferon response. In addition to stimulating NF-κB, NOD1 also binds MAVS to stimulate interferon (IFN) production by activating IRFs. Using HEK293T cells stably containing an IFN-sensitive response element (ISRE)-driven luciferase reporter gene, the effects of compound 1 on several IFN inducers, including NOD1 ligand γ-tri-DAP, poly(I:C), poly(dA:dT), and a RNA virus (Sendai virus) were tested. While compound 1 suppressed ISRE-driven reporter gene activity induced by γ-tri-DAP, no inhibition was observed for the other interferon response stimuli (FIG. 1C). In contrast, the negative control CID-44229067 did not inhibit γ-tri-DAP-induced ISRE reporter gene activity (FIG. 1C). These results further demonstrate the selectivity of the NOD1 inhibitory benzimidazole compounds utilized herein, and also indicate that they act upstream of the divergence of the NF-κB and IFN-dependent pathways activated by NOD1.

Many NLRs form complexes with caspase-1, creating so-called “inflammasomes” responsible for proteolytic processing of inflammatory cytokine interleukin 1-beta (IL-1β). Compound 1 did not inhibit IL-1β secretion induced by various inflammasome activators. (FIGS. 1D and 1G), indicating a lack of promiscuity towards other NLRs.

It was also discovered that compound 1 selectively inhibits responses of primary dendritic cells to NOD1 ligand. To extend the analysis of CID-1088438 beyond immortalized cell lines to primary cells, experiments using ex vivo cultures of human monocyte-derived dendritic cells (DC). DCs were activated with either γ-tri-DAP or lipopolysaccharide (LPS) were performed, in the presence or absence of active compound 1. Compound 1 reduced cell surface expression of co-stimulatory molecules CD83, CD86 and HLA-DR (FIG. 1E) and also inhibited expression of IL-1β, IL-6 and TNF-α. (FIG. 1F) elicited by γ-tri-DAP (but not by LPS), without causing cytoxicity. No significant changes in NOD1 expression levels were observed (FIG. 1F).

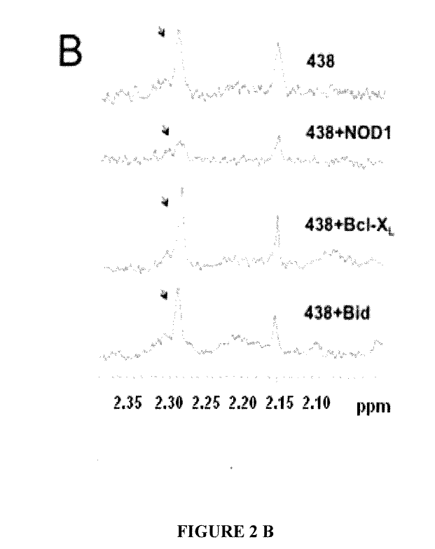

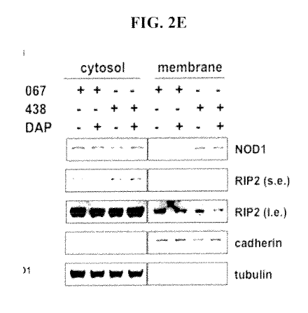

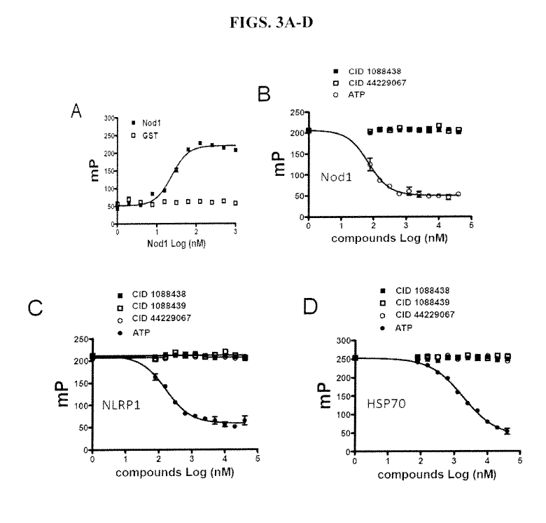

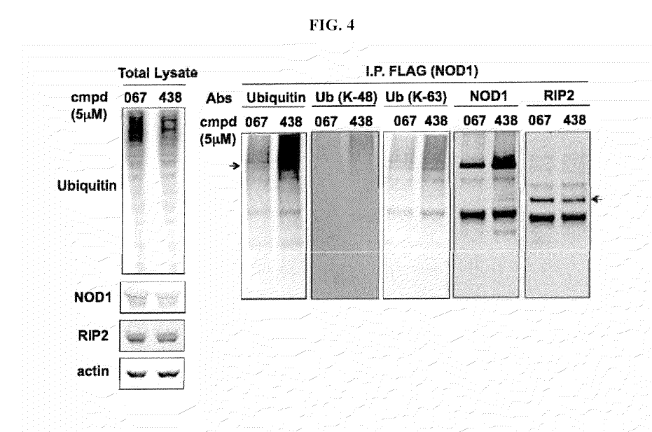

NOD1 activates NF-κB in partnership with various interacting proteins, particularly RIP2, IAPs, and IKKγ/NEMO, where NOD1 binds directly to RIP2, which in turn interacts with IAPs, forming a complex that stimulates IKK activation (Krieg and Reed, 2010). Gene transfection experiments indicated that compound 1 targets NOD1 signaling upstream of RIP2 (FIG. 2A). No significant impact of the compound was observed in cells over-expressing IKKγ/NEMO, MYD88, FLIP, CARD6, APAF1 or NLRC4, demonstrating specificity.

To examine whether compound 1 binds NOD1, recombinant NOD1 protein from human cells were expressed and purified and one-dimensional nuclear magnetic resonance (1D 1H-NMR) spectroscopy was performed as a means to examine ligand binding. The proton (1H) signal intensity derived from compound 1 was suppressed in the presence of NOD1 but not various control proteins such as Bcl-XL and Bid, thereby demonstrating direct interaction between this compound and NOD1 protein (FIG. 2B). However, the spectrum of the inactive negative control (CID 44229067) was also suppressed by NOD1 protein, which indicate that this compound may also bind NOD1, but fails to suppress its cellular activity. Similar results were obtained by affinity selection mass spectrometry. Interestingly, compound 1 did not interfere with ATP binding to recombinant NOD1 protein (FIG. 3).

It was discovered that compound 1 may alter the conformation of NOD1 protein in vitro. For example, in experiments using purified His6-tagged NOD1, addition of compound 1 but not a negative control, markedly increased the relative amount of His6-NOD1 protein that bound to nickel-chelating resin (Ni/NTA) without affecting the binding of other His6-tagged control proteins such as Bcl-XL and Bid (FIG. 2C). Similar results were obtained when compound 1 was added to living cells expressing His6-FLAG-NOD1 protein (FIG. 2D). In contrast, this compound had no observable effect on the efficiency that His6-myc-NOD2 protein was pulled down by Ni/NTA (FIG. 2D). Interestingly, NOD1 ligand γ-tri-DAP also impacted the efficiency with which His6-FLAG-NOD1 protein was pulled down with Ni/NTA, reducing the relative amount of NOD1 protein recovered from cells treated with either inactive or active compounds without changing total levels of His6-FLAG-NOD1 protein in lysates (FIG. 2D). Addition of proteasome-inhibitor MG132 largely negated the effects of both γ-tri-DAP and compounds on His6-FLAG-NOD1 pull-down by Ni/NTA, suggesting a role also for ubiquitination in controlling NOD1 protein conformation. It was also discovered that NOD1 undergoes both lysine 48 (K48) and K63-linked polyubiquitination (FIG. 4).

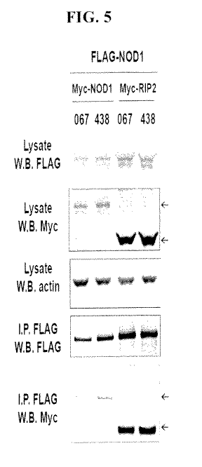

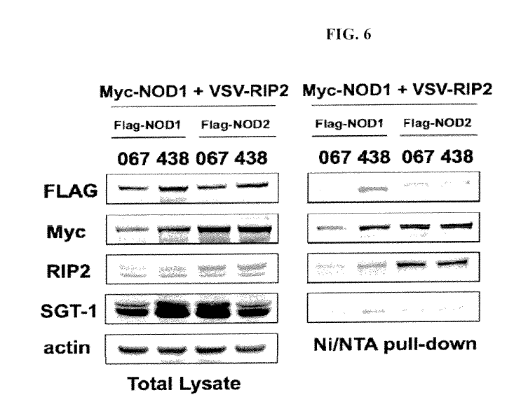

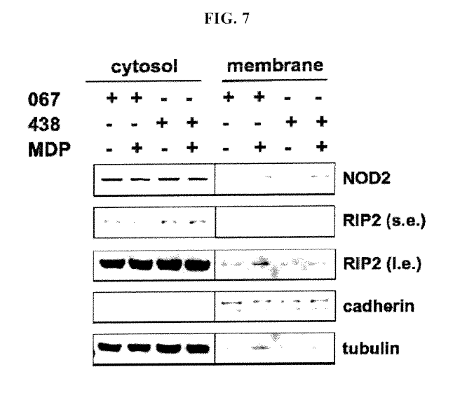

Compound 1 did not interfere with NOD1 association with RIP2, Bid, or SGT-1 under over-expressing conditions (FIGS. 5 and 6). Based on these results, it appears that direct interference with protein-protein interactions may not be responsible for the NOD1 inhibitory mechanism of the utilized compounds.

A subcellular fractionation analysis of MCF-7 cells stably expressing epitope tagged NOD1 (FIG. 2E) or NOD2 (FIG. 7) was performed, which are conveniently detected by immunoblotting using epitope-specific antibodies. Cells were treated with our without the respective NOD1 or NOD2 activators (γ-tri-DAP or MDP), in the presence or absence of compounds. Remarkably, compound 1 (Table 1) induced enrichment of NOD1 protein in the membrane fraction, independently of γ-tri-DAP induction. No changes of NOD2 compartmentalization were observed upon treatment with CID-1088438 (FIG. 7). In contrast, compound 1 reduced membrane localization of RIP2, a NOD1-binding partner required for NF-κB induction. These data are in concordance with reports that migration of RIP2 to the membrane is essential for stimulating NF-κB signaling. It is contemplated that compound 1 alters subcellular targeting of NOD1.

### Cells

Disclosed are cells, such as cells that can be used in the disclosed methods and compositions. For example, disclosed are NOD test cells. NOD test cells are cells that allow assessment of NOD1 and/or NOD2 activation. For example, cells containing an NF-κB-responsive reported construct are useful as NOD test cells. Also disclosed are IL-8 test cells. IL-8 test cells are cells that allow assessment of levels of Interleukin-8 (IL-8). For example, IL-8 test cells can allow assessment of levels of IL-8 affected by test compounds. Such cells can include, for example, a NOD expression construct. Numerous cells and cell lines are known and can be used in the disclosed methods. Useful cells include, for example, vertebrate cells, mammalian cells, rodent cells, primate cells, and human cells.

Also disclosed herein is a cultured cell comprising any of the nucleic acids disclosed herein operably linked to an expression control sequence. The cell can be any cell or cell line, including transformed cells and primary cell lines, that can be used to produce recombinant protein. In some aspects, the cell is a eukaryotic cell. For example, the cell can be a Chinese Hamster Ovary (CHO) cell. CHO cells are a cell line derived from Chinese Hamster ovary cells. The cell can be a HEK 293 cell. The cell can be a HEK 293T cell, HEK293 cells were generated by transformation of human embryonic kidney cell cultures (hence HEK) with sheared adenovirus 5 DNA. The cell can be a SF9 cell. SF9 cells are an insect cell line derived from Spodoptera frugiperda much used for production of recombinant protein. The cell can be a human breast cancer epithelial MCF-7 cell.

In some aspects, the cell is a stem cell. One category of stem cells is a pluripotent embryonic stem cell. A “pluripotent stem cell” as used herein means a cell which can give rise to many differentiated cell types in an embryo or adult, including the germ cells (sperm and eggs). Pluripotent stem cells are also capable of self-renewal. Thus, these cells not only populate the germ line and give rise to a plurality of terminally differentiated cells which comprise the adult specialized organs, but also are able to regenerate themselves. One category of stem cells are cells which are capable of self renewal and which can differentiate into cell types of the mesoderm, ectoderm, and endoderm, but which do not give rise to germ cells, sperm or egg.

Another category of stein cells is an adult stem cell which is any type of stem cell that is not derived from an embryo/fetus. For example, recent studies have indicated the presence of a more primitive cell population in the bone marrow capable of self-renewal as well as differentiation into a number of different tissue types other than blood cells. These multi-potential cells were discovered as a minor component in the CD34-plastic-adherent cell population of adult bone marrow, and are variously referred to as mesenchymal stem cells (MSC) (Pittenger, et al., Science 284:143-147 (1999)) or multi-potent adult progenitor cells (MAYO) cells (Furcht, L. T., et al., U.S. patent publication 20040107453 A1). MSC cells do not have a single specific identifying marker, but have been shown to be positive for a number of markers, including CD29, CD90, CD105, and CD73, and negative for other markers, including CD14, CD3, and CD34. Various groups have reported to differentiate MSC cells into myocytes, neurons, pancreatic beta-cells, liver cells, bone cells, and connective tissue. Another group (Wernet et al., U.S. patent publication 20020164794 A1) has described an unrestricted somatic stem cell (USSC) with multi-potential capacity that is derived from a CD45/CD34 population within cord blood. Typically, these stem cells have a limited capacity to generate new cell types and are committed to a particular lineage, although adult stem cells capable of generating all three cell types have been described (for example, United States Patent Application Publication No 20040107453 by Furcht, et al. published Jun. 3, 2004 and PCT/US02/04652, which are both incorporated by reference at least for material related to adult stem cells and culturing adult stem cells). An example of an adult stem cell is the multipotent hematopoietic stem cell, which forms all of the cells of the blood, such as erythrocytes, macrophages, T and B cells. Cells such as these are often referred to as “pluripotent hematopoietic stem cell” for its pluripotency within the hematopoietic lineage. A pluripotent adult stem cell is an adult stem cell having pluripotential capabilities (See for example, United States Patent Publication no. 20040107453, which is U.S. patent application Ser. No. 10/467,963).

Another category of stem cells is a blastocyst-derived stem cell which is a pluripotent stem cell which was derived from a cell which was obtained from a blastocyst prior to the, for example, 64, 100, or 150 cell stage. Blastocyst-derived stein cells can be derived from the inner cell mass of the blastocyst and are the cells commonly used in transgenic mouse work (Evans and Kaufman, (1981) Nature 292:154-156; Martin, (1981) Proc. Natl. Acad. Sci. 78:7634-7638). Blastocyst-derived stem cells isolated from cultured blastocysts can give rise to permanent cell lines that retain their undifferentiated characteristics indefinitely. Blastocyst-derived stem cells can be manipulated using any of the techniques of modern molecular biology, then re-implanted in a new blastocyst. This blastocyst can give rise to a full term animal carrying the genetic constitution of the blastocyst-derived stem cell. (Misra and Duncan, (2002) Endocrine 19:229-238). Such properties and manipulations are generally applicable to blastocyst-derived stem cells. It is understood blastocyst-derived stem cells can be obtained from pre or post implantation embryos and can be referred to as that there can be pre-implantation blastocyst-derived stem cells and post-implantation blastocyst-derived stem cells respectively.

Pluripotential stem cells can be isolated from fetal material, for example, from gonadal tissues, genital ridges, mesenteries or embryonic yolk sacs of embryos or fetal material. For example, such cells can be derived from primordial germ cells (PGCs). Pluripotential stem cells can also be derived from early embryos, such as blastocysts, testes (fetal and adult), and from other pluripotent stem cells such as ES and EG cells following the methods and using the compositions described herein.

The disclosed cells can lack the cell surface molecules required to substantially stimulate allogeneic lymphocytes in a mixed lymphocyte reaction. For example, the cells can lack the surface molecules required to substantially stimulate CD4+ T-cells in in vitro assessments, or in vivo in allogeneic, syngeneic, or autologous recipients. Preferably, the disclosed cells do not cause any substantial adverse immunological consequences for in vivo applications. For example, the therapeutic cell cultures can lack detectable amounts of at least two, or several, or all of the stimulating proteins HLA-DR, HLA-DP, HLA-DQ, CD80, CD86, and B7-H2, as determined by flow cytometry. Those lacking all of the foregoing are most preferred. Also preferred are therapeutic cell cultures which further lack detectable amounts of one or both of the immuno-modulating proteins HLA-G and CD178, as determined by flow cytometry. Also preferred are therapeutic cell cultures which express detectable amounts of the immuno-modulating protein PD-L2, as determined by flow cytometry. In one embodiment, the therapeutic cell culture does not substantially stimulate a lymphocyte mediated response in vitro, as compared to allogeneic controls in a mixed lymphocyte reaction.

### Constructs and Nucleic Acids

Disclosed are constructs, such as constructs for expression of genes and proteins in cells. Such constructs and cells can be used in the disclosed methods and compositions. For example, disclosed are NF-κB-responsive reporter constructs. NF-κB-responsive constructs are constructs that are expressed when activated by NF-κB. Such constructs can be designed and produced to be generally responsive to NF-κB and such constructs generally will be responsive to NF-κB in a variety of cells and in a variety of conditions. However, it is understood that whether a construct is an N F-κB-responsive construct can depend on the cell and conditions. NF-κB-responsive reporter constructs are NF-κB-responsive constructs that produce a detectable effect or product when expressed. For example, the NF-κB-responsive reported construct can encode a reporter protein.

NF-κB-responsive constructs generally will contain one or more NF-κB-responsive elements. Activated NF-κB is transported to the nucleus where it binds to NF-κB-responsive elements in genes and recruits other proteins that results expression of the NF-κB-responsive genes.

For use in the disclosed methods, the NF-κB-responsive reporter construct can be expressed, for example, under NOD-inducing conditions. NOD-inducing conditions are conditions under which NOD1, NOD2, or both are expressed and/or activated. Activation of NOD1 and/or NOD2 activates the NOD1 and/or NOD2 signaling cascades. NOD activation is one path by which the NF-κB pathway can be activated and by which an NF-κB response can be generated (via expression of NF-κB-responsive genes, for example). Thus, NOD-inducing conditions will also result in the activation of NF-κB, unless an inhibitor of NOD activation and/or NF-κB is present. NOD-inducing conditions include, for example, the presence of a NOD inducer and overexpression of NOD1, NOD2, or both. A NOD inducer is a compound, molecule, composition, etc. that can activate the NOD1 and/or NOD2 signaling pathway. For example, NOD1, NOD2, or both can be directly activated by a NOD inducer. Examples of NOD induciers include Ala-γ Glu-diaminopimelic acid (γ-tri-DAP) and muramyldipeptide (MDP). Overexpression of NOD proteins can overwhelm a cell's normal regulation of NOD activation, resulting in active NOD proteins and activation of one or both of the NOD signaling pathways.

NOD expression and overexpression can be accomplished, for example, by use of a NOD expression construct. A NOD expression construct is a construct encoding NOD1, NOD2, or both, and providing for expression of the NOD gene(s). Expression of the NOD gene(s) in a NOD expression construct can be constitutive, regulatable, inducible, or repressible. The form of expression can be chosen based on the use and needs of the use. NOD1, NOD2, or both can be expressed constitutively in NOD expression constructs. NOD1, NOD2, or both can be expressed via induction in NOD expression constructs.

Myriad vectors, genes, expression control elements, and the like are known and can be used in the disclosed constructs. Generally, expression control elements are nucleic acid sequences that control, affect, enable, specify, etc. expression of operably-linked genes and sequences. Those of skill in the art are aware of such materials and the numerous techniques that can be used to produce constructs, introduce constructs into cells, and assess expression and effects of constructs. For example, disclosed herein is an expression vector comprising an isolated nucleic acid disclosed herein operably linked to an expression control sequence. Thus, disclosed herein is an expression vector comprising the nucleic acid sequence that encodes any of the various genes, proteins, of peptides disclosed herein operably linked to an expression control sequence. Useful expression control elements in clued NF-κB-responsive elements, such as HIV NF-κB-responsive elements.

The expression control sequence can be a tissue specific promoter. Any tissues specific promoter can be used. For example, neural, tumor, and pancreatic specific promoters are disclosed. Examples of some tissue-specific promoters include but are not limited to MUC1, EHA, ACTB, WAP, bHLH-EC2, HOXA-1, Alpha-fetoprotein (AFP), opsin, CR1/2, Fc-γ-Receptor 1 (Fc-γ-R1), MMTVD-LTR, the human insulin promoter, Pdha-2. HOXA-1 is a neuronal tissue specific promoter, and as such, proteins expressed under the control of HOXA-1 are only expressed in neuronal tissue. Sequences for these and other tissue-spec promoters are known in the art and can be found, for example, in Genbank, at the web site pubmed.gov.

The expression control sequence can be an inducible promoter. For example, tetracycline controlled transcriptional activation is a method of inducible expression where transcription is reversibly turned on or off in the presence of the antibiotic tetracycline or one of its derivatives (etc. doxycycline nature, pTet promotes TetR, the repressor, and TetA, the protein that pumps tetracycline antibiotic out of the cell. Two systems named Tet-off and Tet-on are used.

The Tet-off system makes use of the tetracycline transactivator (tTA) protein created by fusing one protein, TetR (tetracycline repressor), found in Escherichia coli bacteria with another protein, VP16, produced by the Herpes Simplex Virus. The tTA protein binds on DNA at a ‘tet’ O operator. Once bound the ‘tet’ O operator will activate a promoter coupled to the ‘tet’ O operator, activating the transcription of nearby gene. Tetracycline derivatives bind tTA and render it incapable of binding to TRE sequences, therefore preventing transactivation of target genes. This expression system is also used in generation of transgenic mice, which conditionally express gene of interest.

The Tet-on system works in the opposite fashion. In that system the rtTA protein is only capable of binding the operator when bound by doxycycline. Thus the introduction of doxycycline to the system initiates the transcription of the genetic product. The tet-on system is sometimes preferred for the faster responsiveness.

Also disclosed for use in the provided compositions and methods are Cre, FRT and ER (estrogen receptor) conditional gene expression systems. In Cre and FRT systems, activation of knockout of the gene is irreversible once recombination is accomplished, while in Tet and ER systems it is reversible. Tet system has very tight control on expression, while ER system is somewhat leaky. However, Tet system, which depends on transcription and subsequent translation of target gene, is not as fast acting as ER system, which stabilizes the already expressed target protein upon hormone administration.

There are a variety of molecules disclosed herein that are nucleic acid based, including for example the nucleic acids that encode, for example genes, proteins, peptides, as well as various functional nucleic acids. The disclosed nucleic acids and constructs can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

i. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (C), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

ii. Sequences

There are a variety of sequences related to genes, peptides and proteins, all of which can be encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at the web site ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

iii. Primers and Probes

Disclosed are compositions including primers and probes, which are capable of interacting with the disclosed nucleic acids, such as the disclosed constructs. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

iv. Expression Systems

The nucleic acids and constructs that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

iv. Viral Promoters and Enhancers

Useful promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that specific regulatory elements can be cloned and used to construct expression vectors that, are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of the construct.

The constructs can comprise lentiviral vectors. Genomes of transgenic mammals comprise integrated transgenes transferred by inventive lentiviral vectors. Lentiviruses belong to the retrovirus family. Retroviruses comprise a diploid RNA genome that is reverse transcribed following infection of a cell to yield a double-stranded DNA intermediate that becomes stably integrated into the chromosomal DNA of the cell. The integrated DNA intermediate is referred to as a provirus and is inherited by the cell's progeny. Wild type retroviral genomes and proviral DNA include gag, pol, and env genes, flanked by two long terminal repeat sequences (LTRs). 5′ and 3′ LTRs comprise sequence elements that promote transcription (promoter-enhancer elements) and polyadenylation of viral RNA. LTRs also include additional cis-acting sequences required for viral replication. Retroviral genomes include sequences needed for reverse transcription and a packaging signal referred to as psi (T) that is necessary for encapsidation (packaging) of a retroviral genome.

The retroviral infective cycle begins when a virus attaches to the surface of a susceptible cell through interaction with cell surface receptor(s) and fuses with the cell membrane. The viral core is delivered to the cytoplasm, where viral matrix and capsid become dismantled, releasing the viral genome. Viral reverse transcriptase (RT) copies the RNA genome into DNA, which integrates into host cell DNA, a process that is catalyzed by the viral integrase (IN) enzyme. Transcription of proviral DNA produces new viral genomes and mRNA from which viral Gag and Gag-Pol polyproteins are synthesized. These polyproteins are processed into matrix (MA), capsid (CA), and nucleocapsid (NC) proteins (in the case of Gag), or the matrix, capsid, protease (PR), reverse transcriptase (RT), and integrase (INT) proteins (in the case of Gag-Pol). Transcripts for other viral proteins, including envelope glycoproteins, are produced via splicing events. Viral structural and replication-related proteins associate with one another, with viral genomes, and with envelope proteins at the cell membrane, eventually resulting in extrusion of a viral particle having a lipid-rich coat punctuated with envelope glycoproteins and comprising a viral genome packaged therein.

Retroviruses are widely used for in vitro and in vivo transfer and expression of heterologous nucleic acids, a process often referred to as gene transfer. For retroviral gene transfer, a nucleic acid sequence (e.g. all or part of a gene of interest), optionally including regulatory sequences such as a promoter, is inserted into a viral genome in place of some of the wild type viral sequences to produce a recombinant viral genome. The recombinant viral genome is delivered to a cell, where it is reverse transcribed and integrated into the cellular genome. Transcription from an integrated sequence may occur from the viral LTR promoter-enhancer and/or from an inserted promoter. If an inserted sequence includes a coding region and appropriate translational control elements, translation results in expression of the encoded polypeptide by the cell. Sequences that are present in the genome of a cell as a result of a process involving reverse transcription and integration of a nucleic acid delivered to the cell (or to an ancestor of the cell) by a retroviral vector are considered a “provirus.” It will be recognized that while such sequences comprise retrovirus derived nucleic acids (e.g., at least a portion of one or more LTRs, sequences required for integration, packaging sequences, etc.), they will typically lack genes for various essential viral proteins and may have mutations or deletions in those viral sequences that they do contain, relative to the corresponding wild type sequences.

Lentiviruses such as HIV differ from the simple retroviruses described above in that their genome encodes a variety of additional proteins such as Vif, Vpr, Vpu, Tat, Rev, and Nef and may also include regulatory elements not found in the simple retroviruses. The genes encoding these proteins overlap with the gag, pol, and env genes. Certain of these proteins are encoded in more than one exon, and their mRNAs are derived by alternative splicing of longer mRNAs. In contrast to simple retroviruses, lentiviruses are able to transduce and productively infect nondividing cells such as resting T cells, dendritic cells, and macrophages. Nondividing cell types of interest include, but are not limited to, cells found in the liver (e.g., hepatocytes), skeletal or cardiac muscle (e.g., myocytes), nervous system (e.g., neurons), retina, and various cells of the hematopoietic system. Lentiviral vectors can transfer genes to hematopoietic stem cells with superior gene transfer efficiency and without affecting the repopulating capacity of these cells (see, e.g., Mautino et al., 2002, AIDS Patient Care STDS 16:11; Somia et al., 2000, J. Virol., 74:4420; Miyoshi et al., 1999, Science, 283:682; and U.S. Pat. No. 6,013,516). Further discussion of retroviruses and lentiviruses is found in Coffin, J., et al. (eds.), Retroviruses, Cold Spring Harbor Laboratory Press, Cold Spring Harbor, 1997, and Fields, B., et al., Fields' Virology, 4.sup.th. ed., Philadelphia: Lippincott Williams & Wilkins, 2001. See also the web site ncbi.nlm.nih.gov/ICTVdb/ICTVdB, accessed Feb. 14, 2006. Useful lentiviral vectors are also described in U.S. Patent Application Publication Nos. 20100172871, 20100137412, 20100062534, 20100028382, 20090325284, 20090217399, 20090214589, 20090148425, 20080233639, 20080226675, 20080200663, 20080167256, 20080131400, 20080120732, 20070036761, 20070025970, 20060281180, 20060269518, 20060134137, 20060019393, 20050266565, 20050244806, and 20050019918.

As used herein, a retroviral vector is considered a “lentiviral vector” if at least approximately 50% of the retrovirus derived LTR and packaging sequences in the vector are derived from a lentivirus and/or if the LTR and packaging sequences are sufficient to allow an appropriately sized nucleic acid comprising the sequences to be reverse transcribed and packaged in a mammalian or avian cell that expresses the appropriate lentiviral proteins. Typically at least approximately 60%, approximately 70%, approximately 80%, approximately 90%, or more of retrovirus derived LTR and packaging sequences in a vector are derived from a lentivirus. For example, LTR and packaging sequences may be at least approximately 50%, approximately 60%, approximately 70%, approximately 80%, approximately 90%, or identical to lentiviral LTR and packaging sequences. In certain embodiments of the invention between approximately 90 and approximately 100% of the LTR and packaging sequences are derived from a lentivirus. For example, the LTR and packaging sequences may be between approximately 90% and approximately 100% identical to lentiviral LTR and packaging sequences.

v. Markers

The viral vectors can include nucleic acid sequence encoding a marker product. This marker product is used to determine if the gene has been delivered to the cell and once delivered is being expressed. Preferred marker genes are the E. coli lacZ gene, which encodes β-galactosidase, and green fluorescent protein. Markers can also serve as reporters.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival non-supplemented media.

The second category is dominant selection which refers to a selection scheme used in any cell type and does not require the use of a mutant cell line. These schemes typically use a drug to arrest growth of a host cell. Those cells which have an appropriate gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### Compositions Including Carriers

The disclosed compositions comprising the disclosed compounds can be combined, conjugated or coupled with or to carriers, including pharmaceutically acceptable carriers and other compositions to aid administration, delivery or other aspects of the inhibitors and their use. For convenience, such composition will be referred to herein as carriers. Carriers can, for example, be a small molecule, pharmaceutical drug, fatty acid, detectable marker, conjugating tag, nanoparticle, or enzyme.

The disclosed compositions can be used therapeutically in combination with a pharmaceutically acceptable carrier. By “pharmaceutically acceptable” is meant a material that is not biologically or otherwise undesirable, i.e., the material can be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds can be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, platonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The carrier molecule can be covalently linked to the disclosed compounds. The carrier molecule can be linked to the amino terminal end of proteins and peptides. The carrier molecule can be linked to the carboxy terminal end of proteins and peptides. The carrier molecule can be linked to an amino acid within proteins and peptides. The disclosed compositions can further comprise a linker connecting the carrier molecule and disclosed inhibitors. The disclosed compounds can also be conjugated to a coating molecule such as bovine serum albumin (BSA) (see Tkachenko et al., (2003) J Am Chem Soc., 125, 4700-4701) that can be used to coat microparticles, nanoparticles of nanoshells with the inhibitors.

i. Nanoparticles, Microparticles, and Microbubbles

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels. See, for example, Y. Lin, et al., Appl. Phys Lett. 2002, 81, 3134; D. Wang, et al., Chem. Mater. 2003, 15, 2724; X. Gao, et al., J. Biomed. Opt. 2002, 7, 532; M. Han, et al., Nature Biotechnology. 2001, 19, 631; V. M. Pai, et al., Mag. & Magnetic Mater. 1999, 194, 262, each of which is incorporated by reference in its entirety. Both the photostability of the chromophores and the monodispersity of the microspheres can be important.

Nanoparticles, such as, for example, silica nanoparticles, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

For example, the disclosed compositions comprising the disclosed compounds can be immobilized on silica nanoparticles (SNPs). SNPs have been widely used for biosensing and catalytic applications owing to their favorable surface area-to-volume ratio, straightforward manufacture and the possibility of attaching fluorescent labels, magnetic nanoparticles (Yang, H. H. et al. 2005) and semiconducting nanocrystals (Lin, Y. W., et al. 2006).

The nanoparticle can also be, for example, a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells.

Targeting molecules can be attached to the disclosed compositions and/or carriers. For example, the targeting molecules can be antibodies or fragments thereof, ligands for specific receptors, or other proteins specifically binding to the surface of the cells to be targeted.

ii. Liposomes

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through tilters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum, Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

Fatty acids (i.e., lipids) that can be conjugated to the provided compositions include those that allow the efficient incorporation of the proprotein convertase inhibitors into liposomes. Generally, the fatty acid is a polar lipid. Thus, the fatty acid can be a phospholipid. The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, diolcoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylscrine, palmitoylolcoylphosphatidylethanolamine, palmitoyloleoylphophatidylglycerol, palmitoylolcoylphosphatidic acid, palmitelaidoyloleoylphosphatidylcholine palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinolcoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

### Combination Therapeutics

Also disclosed herein are compositions that comprise the utilized compounds and any known or newly discovered agents that can be administered systemically or to the site of a inflammation, cardiovascular disease, cancer, neurodegeneration, or metabolic disregulation. For example, the disclosed compositions can further comprise one or more of classes of the following agents: antibiotics (e.g. Aminoglycosides, Cephalosporins, Chloramphenicol, Clindamycin, Erythromycins, Fluoroquinolones, Macrolides, Azolides, Metronidazole, Penicillin's, Tetracycline's, Trimethoprim-sulfamethoxazole, Vancomycin), steroids (e.g. Andranes (e.g. Testosterone). Cholestanes (e.g. Cholesterol), Cholic acids (e.g. Cholic acid), Corticosteroids (e.g. Dexamethasone), Estraenes (e.g. Estradiol), Pregnanes (e.g. Progesterone), narcotic and non-narcotic analgesics (e.g. Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g. Alclofenac; Alclometasone Dipropionate; Algestone Acetonide; alpha Amylase; Amcinafal, Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Decanoate; Deflazacort; Delatestryl; Depo-Testosterone; Desonide; Desoximetasone; Dexamcthasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lolemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Mesterolone; Methandrostenolone; Methenolone; Methenolone Acetate; Methylprednisolone Suleptanate; Momiflumate; Nabumetone; Nandrolone; Naproxen; Naproxen Sodium; Naproxol; Nimazone; Olsalazine Sodium; Orgotein; Orpanoxin; Oxandrolane; Oxaprozin; Oxyphenbutazone, Oxymetholone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic. Acid; Proquazone; Proxazole; Proxazole Citrate; Rimexolone; Romazarit; Salcolex; Salnacedin; Salsalate; Sanguinarium Chloride; Seclazone; Sermetacin; Stanozolol; Sudoxicam; Sulindac; Suprofen; Talmetacin; Talniflumate; Talosalate; Tebufelone; Tenidap; Tenidap Sodium; Tenoxicam; Tesicam; Tesimide; Testosterone; Testosterone Blends; Tetrydamine; Tiopinac; Tixocortol Pivalate; Tolmetin; Tolmetin Sodium; Triclonide; Triflumidate; Zidometacin; Zomepirac Sodium), or anti-histaminic agents Ethanolamines (like diphenhydramine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

The disclosed composition can further comprise one or more additional radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al. 2003; Zhang et al., 2004).

The disclosed composition can further comprise Levodopa. The most widely used form of treatment is L-dopa in various forms. L-dopa is transformed into dopamine in the dopaminergic neurons by L-aromatic amino acid decarboxylase (often known by its former name dopa-decarboxylase). However, only 1-5% of L-DOPA enters the dopaminergic neurons. The remaining L-DOPA is often metabolised to dopamine elsewhere, causing a wide variety of side effects. Due to feedback inhibition, L-dopa results in a reduction in the endogenous formation of L-dopa, and so eventually becomes counterproductive.

The disclosed composition can further comprise Carbidopa or Benserazide. Carbidopa or Benserazide are dopa decarboxylase inhibitors. They help to prevent the metabolism of L-dopa before it reaches the dopaminergic neurons and are general given as combination preparations of carbidopa/levodopa (co-careldopa BAN) co-careldopa combined L-dopa and carbidopa in fixed ratios in such branded products of Sinemetand Parcopa and Benserazide/levodopa (co-beneldopa BAN) as Madopar. There are also controlled release versions of Sinemet and Madopar that spread out the effect of the L-dopa. Duodopa is a combination of levodopa and carbidopa, dispersed as a viscous gel. Using a patient-operated portable pump, the drug is continuously delivered via a tube directly into the upper small intestine, where it is rapidly absorbed.

The disclosed composition can further comprise Talcopone. Talcopone inhibits the COMT enzyme, thereby prolonging the effects of L-dopa, and so has been used to complement L-dopa. A similar drug, entacapone, has similar efficacy and has not been shown to cause significant alterations of liver function. Stalevo contains Levodopa, Carbidopa and Entacopone.

The disclosed composition can further comprise the dopamine-agonists bromocriptine (Parlodel), pergolidc (Permax), pramipexole (Mirapex), ropinirole (Requip), cabergoline (Cabaser), apomorphine (Apokyn), or lisuride (Revanil). Dopamine agonists initially act by stimulating some of the dopamine receptors.

The disclosed composition can further comprise an MAO-B inhibitor. For example, selegiline (Eldepryl) and rasagiline (Azilect) reduce the symptoms by inhibiting monoamine oxidase-B (MAO-B), which inhibits the breakdown of dopamine secreted by the dopaminergic neurons. By-products of selegiline include amphetamine and methamphetamine, which can cause side effects such as insomnia.

The disclosed composition can further comprise a nucleic acid encoding glutamic acid decarboxylase (GAD), which catalyses the production of a neurotransmitter called GABA. GABA acts as a direct inhibitor on the overactive cells in the STN.

The disclosed compositions can further comprise glial-derived neurotrophic factor (GDNF). Via a series of biochemical reactions, GDNF stimulates the formation of L-dopa.

The disclosed compositions can further comprise an acetylcholinesterase inhibitor. Acetylcholinesterase inhibitors reduce the rate at which acetylcholine (ACh) is broken down and hence increase the concentration of ACh in the brain (combatting the loss of ACh caused by the death of the cholinergin neurons). Examples currently marketed include donepezil (Aricept, Eisai and Pfizer), galantamine (Razadyne, Ortho-McNeil Neurologics, US) and rivastigmine (Exelon and Exelon Patch, Novartis). Donepezil and galantamine are taken orally. Rivastigmine has oral forms and a once-daily transdermal patch.

The disclosed compositions can further comprise memantine (Namenda, Forest Pharmaceuticals, Axura, Merz GMBh, Ebixa, H. Lundbeck, and Akatinol). Memantine is a novel NMDA receptor antagonist, and has been shown to be moderately clinically efficacious.

The disclosed compositions can further comprise one or more cells. The cell can be a stem cell. The stem cell can be a pluripotent stem cell. The cell can be a progenitor cell. The cell can be a neural progenitor cell. The cell can be a stent cell capable of differentiating into a neural cell. Thus, the disclosed compositions can further comprise stem cells treated with factors to induce differentiation into neural cells. Other such cells known in the art for treating neurodegenerative disease or delivery of compositions to the brain are contemplated herein.

### Administration and Treatment

The disclosed compounds and compositions can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

Oral compositions can include as excipients or carriers, binders, lubricants, tillers, disintegrates, and the like that are well known to the skilled artisan.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the severity of the allergic disorder being treated, the particular nucleic acid or vector used, its mode of administration and the like. An appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of a composition comprising the disclosed compound used alone might range from about 1 μg/kg to up to 100 mg/kg of body weight or more per day, depending on the factors mentioned above.

Following administration of a disclosed composition, the efficacy of the therapeutic compound can be assessed in various ways well known to the skilled practitioner. For instance, one of ordinary skill in the art will understand that a composition disclosed herein is efficacious in treating or inhibiting Alzheimer's disease in a subject by observing that the composition reduces amyloid beta or prevents a further increase in plaque formation. Other indicators of therapeutic efficacy disclosed herein can be measured by methods that are known in the art, for example, using polymerase chain reaction assays to detect the presence of nucleic acid or antibody assays to detect the presence of protein in a sample (e.g., but not limited to, blood) from a subject or patient, or by measuring the level of circulating levels in the patient. Efficacy of the administration of the disclosed composition may also be determined by routine diagnostic means. For example, efficacy of the disclosed compositions for treating diabetes can be determined by monitoring blood sugar.

The compositions disclosed herein may be administered prophylactically to patients or subjects who are at risk for inflammation, neurodegenerative disease, cardiovascular disease, or diabetes or who have been newly diagnosed with inflammation, neurodegenerative disease, cardiovascular disease, or diabetes.

The disclosed compositions and methods can also be used for example as tools to isolate and test new drug candidates for a variety of inflammation related diseases, neurodegenerative disease, cardiovascular disease, or diabetes related diseases.

In some embodiments, the disease treated according to the methods provided here, include without limitation asthma, inflammatory bowel disease, and other inflammatory disorders. In some embodiments, the other inflammatory disorder includes without limitation, Crohn's disease, sarcoidosis, allergy, and multiple sclerosis. In another embodiment, the disease treated is an infectious disease.

Also provided herein are treatment methods wherein the compounds utilized herein are administered in combination with another agent. Administration in “combination” refers to the administration of the two agents (i.e. a an agent utilized herein, for example, of Formula A, I, II, III, and IV and a second agent) in any manner in which the pharmacological effects of both are manifest in the patient at the same time. Thus, administration in combination does not require that a single pharmaceutical composition, the same dosage form, or even the same route of administration be used for administration of both the agents or that the two agents be administered at precisely the same time.

i. Inflammation

Examples of inflammatory diseases include, but are not limited to, chronic inflammatory diseases and acute inflammatory diseases. According to some aspects, the disclosed compounds can be used to treat chronic inflammatory disease, such as colitis. According to some aspects, the disclosed compounds can be used to treat acute inflammatory disease, such as asthma. According to some aspects, the disclosed compounds can be used to treat an inflammation associated with hypersensitivity. Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH. According to some aspects of the disclosed compounds, the disclosed compounds can be used to treat Type I or immediate hypersensitivity, such as asthma.

Examples of Type II hypersensitivity include, but are not limited to, rheumatoid diseases, rheumatoid autoimmune diseases, rheumatoid arthritis (Krenn V. et al., Histol Histopathol July 2000; 15 (3): 791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2): 49), sclerosis, systemic sclerosis (Renaudineau Y. et al., Clin Diagn Lab Immunol. March 1999; 6 (2): 156); Chan O T. et al., Immunol Rev June 1999; 169: 107), glandular diseases, glandular autoimmune diseases, pancreatic autoimmune diseases, diabetes, Type I diabetes (Zimmet P. Diabetes Res Clin Pract October 1996; 34 Suppl:S125), thyroid diseases, autoimmune thyroid diseases, Graves' disease (Orgiazzi J. Endocrinol Metab Clin North Am June 2000; 29 (2): 339), thyroiditis, spontaneous autoimmune thyroiditis (Braley-Mullen H. and Yu S, J Immunol Dec. 15, 2000; 165 (12): 7262), Hashimoto's thyroiditis (Toyoda N. et al. Nippon Rinsho August 1999; 57 (8): 1810), myxedema, idiopathic myxedema (Mitsuma T. Nippon Rinsho. August 1999; 57 (8): 1759); pancreatitis, autoimmune reproductive diseases, ovarian diseases, ovarian autoimmunity (Garza K M. et al., J Reprod Immunol. February 1998; 37 (2). 87), autoimmune anti-sperm infertility (Diekman A B. et al., Am J Reprod Immunol. March 2000; 43 (3): 134), repeated fetal loss (Tincani A. et al., Lupus 1998; 7 Suppl 2: S107-9), neurodegenerative diseases, neurological diseases, neurological autoimmune diseases, multiple sclerosis (Cross A H. et al., J Neuroimmunol Jan. 1, 2001; 112 (1-2): 1), Alzheimer's disease (Oron L. et al. J Neural Transm Suppl. 1997; 49: 77), myasthenia gravis (Infante A J. And Kraig E, Int Rev Immunol 1999; 18 (1-2): 83), motor neuropathies (Kornberg A J. J Clin Neurosci. May 2000; 7 (3): 191), Guillain-Barre syndrome, neuropathies and autoimmune neuropathies (Kusunoki S. Am J Med Sci. April 2000; 319 (4): 234), myasthenic diseases, Lambert-Eaton myasthenic syndrome (Takamori M. Am J Med Sci. April 2000; 319 (4): 204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) January 2000; 156 (1): 23); neuropathies, dysimmune neuropathies (Nobile-Orazio E. et al., Electroencephalogr Clin Neurophysiol Suppl 1999; 50: 419); neuromyotonia, acquired neuromyotonia, arthrogryposis multiplex congenita (Vincent A. et al., Ann N Y Acad Sci. May 13, 1998; 841: 482), cardiovascular diseases, cardiovascular autoimmune diseases, atherosclerosis (Matsuura E. et al., Lupus. 1998; 7 Suppl 2: S135), myocardial infarction (Vaarala O. Lupus. 1998; 7 Suppl 2: S132), thrombosis (Tincani A. et al., Lupus 1998; 7 Suppl 2: S107-9), granulomatosis, Wegener's granulomatosis, arteritis, Takayasu's arteritis and Kawasaki syndrome (Praprotnik S. et al., Wien Klin Wochenschr Aug. 25, 2000; 112 (15-16): 660); anti-factor VIII autoimmune disease (Lacroix-Desmazes S. et al., Semin Thromb Hemost. 2000; 26 (2): 157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). May 2000; 151 (3): 178); antiphospholipid syndrome (Flamholz R. et al., J Clin Apheresis 1999; 14 (4): 171); heart failure, agonist-like beta-adrenoceptor antibodies in heart failure (Wallukat G. et al., Am J Cardiol. Jun. 17, 1999; 83 (12A): 75H), thrombocytopenic purpura (Moccia F. Ann Ital Med Int. April-June 1999; 14 (2): 114); hemolytic anemia, autoimmune hemolytic anemia (Efremov D G. et al., Leuk Lymphoma January 1998; 28 (3-4): 285), gastrointestinal diseases, autoimmune diseases of the gastrointestinal tract, intestinal diseases, chronic inflammatory intestinal disease (Garcia Herola A. et al., Gastroenterol Hepatol. January 2000; 23 (1): 16), celiac disease (Landau Y E. and Shoenfeld Y. Harefuah Jan. 16, 2000; 138 (2): 122), autoimmune diseases of the musculature, myositis, autoimmune myositis, Sjogren's syndrome (Feist E. et al., Int Arch Allergy Immunol September 2000; 123 (1): 92); smooth muscle autoimmune disease (Zauli D. et al., Biomed Pharmacother June 1999; 53 (5-6): 234), hepatic diseases, hepatic autoimmune diseases, autoimmune hepatitis (Manns M P. J Hepatol August 2000; 33 (2): 326) and primary biliary cirrhosis (Strassburg C P. et al., Eur J Gastroenterol Hepatol. June 1999; 11 (6): 595).

According to some aspects, the disclosed method can be used to treat Type IV or T lymphocyte mediated hypersensitivity. For example, the disclosed method can be used to treat Type IV or T lymphocyte mediated hypersensitivity such as DTH.

Examples of Type IV or T cell mediated hypersensitivity, include, but are not limited to, rheumatoid diseases, rheumatoid arthritis (Tisch R, McDevitt H O. Proc Natl Acad Sci USA Jan. 18, 1994; 91 (2): 437), systemic diseases, systemic autoimmune diseases, systemic lupus erythematosus (Datta S K., Lupus 1998; 7 (9): 591), glandular diseases, glandular autoimmune diseases, pancreatic diseases, pancreatic autoimmune diseases, Type 1 diabetes (Castano L. and Eisenbarth G S. Ann. Rev. Immunol. 8: 647); thyroid diseases, autoimmune thyroid diseases, Graves' disease (Sakata S. et al., Mol Cell Endocrinol March 1993; 92 (1): 77); ovarian diseases (Garza K M. et al., J Reprod Immunol February 1998; 37 (2): 87), prostatitis, autoimmune prostatitis (Alexander R B. et al., Urology December 1997; 50 (6): 893), polyglandular syndrome, autoimmune polyglandular syndrome, Type I autoimmune polyglandular syndrome (Hara T. et al., Blood. Mar. 1, 1991; 77 (5): 1127), neurological diseases, autoimmune neurological diseases, multiple sclerosis, neuritis, optic neuritis (Soderstrom M. et al., J Neurol Neurosurg Psychiatry May 1994; 57 (5): 544), myasthenia gravis (Oshima M. et al., Eur J Immunol December 1990; 20 (12): 2563), stiffman syndrome (Hiemstra H S. et al., Proc Natl Acad Sci USA Mar. 27, 2001; 98 (7): 3988), cardiovascular diseases, cardiac autoimmunity in Chagas' disease (Cunha-Neto E. et al., J Clin Invest Oct. 15, 1996; 98 (8): 1709), autoimmune thrombocytopenic purpura (Semple J W. et al., Blood May 15, 1996; 87 (10): 4245), anti-helper T lymphocyte autoimmunity (Caporossi A P. et al., Viral Immunol 1998; 11 (I): 9), hemolytic anemia (Sallah S. et al., Ann Hematol March 1997; 74 (3): 139), hepatic diseases, hepatic autoimmune diseases, hepatitis, chronic active hepatitis (Franco A. et al., Clin Immunol Immunopathol March 1990; 54 (3): 382), biliary cirrhosis, primary biliary cirrhosis (Jones D E. Clin Sci (Colch) November 1996; 91 (5): 551), nephric diseases, nephric autoimmune diseases, nephritis, interstitial nephritis (Kelly C J. J Am Soc Nephrol August 1990; 1 (2): 140), connective tissue diseases, ear diseases, autoimmune connective tissue diseases, autoimmune ear disease (Yoo T J. et al., Cell Immunol August 1994; 157 (1): 249), disease of the inner ear (Gloddek B. et al., Ann N Y Acad Sci Dec. 29, 1997; 830: 266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption. Examples of types of T lymphocyte mediating hypersensitivity include, but are not limited to, helper T lymphocytes and cytotoxic T lymphocytes. Examples of helper T lymphocyte-mediated hypersensitivity include, but are not limited to, T.sub.h1 lymphocyte mediated hypersensitivity and T.sub.h2 lymphocyte mediated hypersensitivity.

According to some aspects, the disclosed compounds can be used to treat an inflammation associated with an autoimmune disease. Examples of autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases. According to some aspects, the disclosed compounds can be used to treat autoimmune gastrointestinal diseases, such as colitis.

Examples of autoimmune cardiovascular diseases include, but are not limited to atherosclerosis (Matsuura E. et al., Lupus. 1998; 7 Suppl 2: S135), myocardial infarction (Vaarala O. Lupus. 1998; 7 Suppl 2: S132), thrombosis (Tincani A. et al., Lupus 1998; 7 Suppl 2: S107-9), Wegener's granulomatosis, Takayasu's arteritis, Kawasaki syndrome (Praprotnik S. et al., Wien Klin Wochenschr Aug. 25, 2000; 112 (15-16): 660), anti-factor VIII autoimmune disease (Lacroix-Desmazes S. et al., Semin Thromb Hemost. 2000; 26 (2): 157), necrotizing small vessel vasculitis, microscopic polyangiitis, Churg and Strauss syndrome, pauci-immune focal necrotizing and crescentic glomerulonephritis (Noel L H. Ann Med Interne (Paris). May 2000; 151 (3): 178), antiphospholipid syndrome (Flamholz R. et al., J Clin Apheresis 1999; 14 (4): 171), antibody-induced heart failure (Wallukat G. et al., Am J Cardiol. Jun. 17, 1999; 83 (12A): 7511), thrombocytopenic purpura (Moccia F. Ann Ital Med Int. April-June 1999; 14 (2): 114; Semple J W. et al., Blood May 15, 1996; 87 (10): 4245), autoimmune hemolytic anemia (Efremov D G. et al., Leuk Lymphoma January 1998; 28 (3-4): 285; Sallah S. et al., Ann Hematol March 1997; 74 (3): 139), cardiac autoimmunity in Chagas' disease (Cunha-Neto E. et al., J Clin Invest Oct. 15, 1996; 98 (8): 1709) and anti-helper T lymphocyte autoimmunity (Caporossi A P. et al., Viral Immunol 1998; 11 (1): 9).

Examples of autoimmune rheumatoid diseases include, but are not limited to rheumatoid arthritis (Krenn V. et al., Histol Histopathol July 2000; 15 (3): 791: Tisch R, McDevitt H O. Proc Natl Acad Sci units S A Jan. 18, 1994; 91 (2): 437) and ankylosing spondylitis (Jan Voswinkel. et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome, diseases include, but are not limited to autoimmune diseases of the pancreas, Type 1 diabetes (Castano L. and Eisenbarth G S. Ann. Rev. Immunol. 8: 647; Zimmet P. Diabetes Res Clin Pract October 1996; 34 Suppl: S125), autoimmune thyroid diseases. Graves' disease (Orgiazzi J. Endocrinol Metab Clin North Am June 2000; 29 (2): 339; Sakata S. et al., Mol Cell Endocrinol March 1993; 92 (1): 77), spontaneous autoimmune thyroiditis (Braley-Mullen H. and Yu S, J Immunol Dec. 15, 2000; 165 (12): 7262). Hashimoto's thyroiditis (Toyoda N. et al., Nippon Rinsho August 1999; 57 (8): 1810), idiopathic myxedema (Mitsuma T. Nippon Rinsho. August 1999; 57 (8): 1759), ovarian autoimmunity (Garza K M. et al., J Reprod Immunol February 1998; 37 (2): 87), autoimmune anti-sperm infertility (Diekman A B. et al., Am J Reprod Immunol. March 2000; 43 (3): 134), autoimmune prostatitis (Alexander R B. et al., Urology December 1997; 50 (6): 893) and Type I autoimmune polyglandular syndrome (Hara T. et al., Blood. Mar. 1, 1991; 77 (5): 1127).

Examples of autoimmune gastrointestinal diseases include, but are not limited to, chronic inflammatory intestinal diseases (Garcia Herola A. et al., Gastroenterol Hepatol. January 2000; 23 (1): 16), celiac disease (Landau Y E. and Shoenfeld Y. Harefuah Jan. 16, 2000; 138 (2): 122), pancreatitis, colitis, ileitis, Crohn's disease, and ulcerative colitis.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol March 1990; 54 (3) 382), primary biliary cirrhosis (Jones D E. Clin Sci (Colch) November 1996; 91 (5): 551; Strassburg C P. et al., Eur J Gastroenterol Hepatol. June 1999; 11 (6): 595) and autoimmune hepatitis (Manns M P. J Hepatol August 2000; 33 (2): 326).

Examples of autoimmune neurological diseases include, but are not limited to, multiple sclerosis (Cross A H. et al., J Neuroimmunol Jan. 1, 2001; 112 (1-2): 1), Alzheimer's disease (Oron L. et al., J Neural Transm Suppl. 1997; 49: 77), myasthenia gravis (Infante A J. And Kraig F, Int Rev Immunol 1999; 18 (1-2): 83: Oshima M. et al., Eur J Immunol December 1990; 20 (12): 7563), neuropathies, motor neuropathies (Komberg A J. J Clin Neurosci. May 2000; 7 (3): 191); Guillain-Barre syndrome and autoimmune neuropathies (Kusunoki S. Am J Med Sci. April 2000; 319 (4): 234), myasthenia, Lambert-Eaton myasthenic syndrome (Takamori M. Am J Med Sci. April 2000; 319 (4): 204); paraneoplastic neurological diseases, cerebellar atrophy, paraneoplastic cerebellar atrophy and stiff-man syndrome (Hiemstra H S. et al., Proc Natl Acad Sci units SA Mar. 27, 2001; 98 (7): 3988); non-paraneoplastic stiff man syndrome, progressive cerebellar atrophies, encephalitis, Rasmussen's encephalitis, amyotrophic lateral sclerosis, Sydeham chorea. Gilles de la Tourette syndrome and autoimmune polyendocrinopathies (Antoine J C. and Honnorat J. Rev Neurol (Paris) January 2000; 156 (1): 23); dysimmune neuropathies (Nobile-Orazio E. et al., Electroencephalogr Clin Neurophysiol Suppl 1999; 50: 419); acquired neuromyotonia, arthrogryposis multiplex congenita (Vincent A. et al., Ann N Y Acad Sci. May 13, 1998; 841: 482), neuritis, optic neuritis (Soderstrom M. et al., J Neurol Neurosurg Psychiatry May 1994; 57 (5): 544) and neurodegenerative diseases.

Examples of autoimmune muscular diseases include, but are not limited to, myositis, autoimmune myositis and primary Sjogren's syndrome (Feist E. et al., Int Arch Allergy Immunol September 2000; 123 (1): 92) and smooth muscle autoimmune disease (Zauli D. et al., Biomed Pharmacother June 1999; 53 (5-6): 234).

Examples of autoimmune nephric diseases include, but are not limited to, nephritis and autoimmune interstitial nephritis (Kelly C J. J Am Soc Nephrol August 1990; 1 (2): 140).

Examples of autoimmune diseases related to reproduction include, but are not limited to, repeated fetal loss (Tincani A. et al., Lupus 1998; 7 Suppl 2: S107-9).

Examples of autoimmune connective tissue diseases include, but are not limited to, ear diseases, autoimmune ear diseases (Yoo T J. et al., Cell Immunol August 1994; 157 (1): 249) and autoimmune diseases of the inner ear (Gloddek B. et al., Ann N Y Acad Sci Dec. 29, 1997; 830: 266).

Examples of autoimmune systemic diseases include, but are not limited to, systemic lupus erythematosus (Erikson J. et al., Immunol Res 1998; 17 (1-2): 49) and systemic sclerosis (Renaudineau Y. et al., Clin Diagn Lab Immunol. March 1999; 6(2): 156); Chan O T. et al., Immunol Rev June 1999; 169: 107).

According to some aspects of the disclosed method, the disclosed compounds can be used to treat an inflammation associated with infectious diseases. Examples of infectious diseases include, hut are not limited to, chronic infectious diseases, subacute infectious diseases, acute infectious diseases, viral diseases, bacterial diseases, protozoan diseases, parasitic diseases, fungal diseases, mycoplasma diseases and prion diseases.

According to some aspects of the disclosed method, the disclosed compounds can be used to treat an inflammation associated with a disease associated with transplantation of a graft. Examples of diseases associated with transplantation of a graft include, but are not limited to, graft rejection, chronic graft rejection, subacute graft rejection, hyperacute graft rejection, acute graft rejection and graft versus host disease. Types of grafts whose rejection can be treated by the disclosed method include, but are not limited to, syngeneic grafts, allografts and xenografts. According to some aspects, the disclosed compounds can be used to treat allograft rejection. Examples of grafts include cellular grafts, tissue grafts, organ grafts and appendage grafts. Examples of cellular grafts include, but are not limited to, stem cell grafts, progenitor cell grafts, hematopoietic cell grafts, embryonic cell grafts and a nerve cell grafts. Examples of tissue grafts include, but are not limited to, skin grafts, bone grafts, nerve grafts, intestine grafts, corneal grafts, cartilage grafts, cardiac tissue grafts, cardiac valve grafts, dental grafts, hair follicle grafts and muscle grafts. Examples of organ grafts include, but are not limited to, kidney grafts, heart grafts, skin grafts, liver grafts, pancreatic grafts, lung grafts and intestine grafts. Examples of appendage grafts include, but are not limited to, arm grafts, leg grafts, hand grafts, foot grafts, finger grafts, toe grafts and sexual organ grafts. According to some aspects, the disclosed compounds can be used to treat kidney allograft rejection.

According to some aspects of the disclosed method, the disclosed compounds can be used to treat inflammation associated with allergic diseases. Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy. For example, the disclosed compounds can be used, according to the disclosed method, to treat asthma.

According to some aspects of the disclosed method, the disclosed compounds can be used to treat inflammations associated with neurodegenerative diseases. According to some aspects of the disclosed method, the disclosed compounds can be used to treat inflammations associated with cardiovascular diseases. According to some aspects of the disclosed method, the disclosed compounds can be used to treat inflammations associated with gastrointestinal diseases. Examples of gastrointestinal diseases include, but are not limited to, the examples of antibody-mediated gastrointestinal diseases listed hereinabove, the examples of T lymphocyte-mediated gastrointestinal diseases listed hereinabove, the examples of autoimmune gastrointestinal diseases listed hereinabove and hemorrhoids. According to some aspects of the disclosed method, the disclosed compounds can be used to treat colitis.

According, to some aspects of the disclosed method, the disclosed compounds can be used to treat inflammations associated with neurodegenerative diseases. According to some aspects of the disclosed method, the disclosed compounds can be used to treat inflammations associated with tumors. Examples of tumors include, but are not limited to, malignant tumors, benign tumors, solid tumors, metastatic tumors and non-solid tumors. According to some aspects of the disclosed method, the disclosed compounds can be used to treat inflammation associated with septic shock.

According to some aspects of the disclosed method, the disclosed compounds can be used to treat inflammation associated with anaphylactic shock. According to some aspects of the disclosed method, the disclosed compounds can be used to treat inflammation associated with toxic shock syndrome. According to some aspects of the disclosed method, the disclosed compounds can be used to treat inflammation associated with cachexia. According to some aspects of the disclosed method, the disclosed compounds can be used to treat inflammation associated with necrosis. According to some aspects of the disclosed method, the disclosed compounds can be used to treat inflammation associated with gangrene.

According to some aspects of the disclosed method, the disclosed compounds can be used to treat inflammations associated with prosthetic implants. Examples of prosthetic implants include, but are not limited to, breast implants, silicone implants, dental implants, penile implants, cardiac implants, artificial joints, bone fracture repair devices, bone replacement implants, drug delivery implants, catheters, pacemakers, respirator tubes and stents.

According to some aspects of the disclosed method, the disclosed compounds can be used to treat inflammation associated with menstruation. According to some aspects of the disclosed method, the disclosed compounds can be used to treat inflammations associated with ulcers. Examples of ulcers include, but are not limited to, skin ulcers, bed sores, gastric ulcers, peptic ulcers, buccal ulcers, nasopharyngeal ulcers, esophageal ulcers, duodenal ulcers, ulcerative colitis and gastrointestinal ulcers.

According to some aspects of the disclosed method, the disclosed compounds can be used to treat inflammations associated with injuries. Examples of injuries include, but are not limited to, abrasions, bruises, cuts, puncture wounds, lacerations, impact wounds, concussions, contusions, thermal burns, frostbite, chemical burns, sunburns, dessications, radiation burns, radioactivity burns, smoke inhalation, torn muscles, pulled muscles, torn tendons, pulled tendons, pulled ligaments, torn ligaments, hyperextensions, torn cartilage, bone fractures, pinched nerves and a gunshot wounds.

According to some aspects of the disclosed method, the disclosed compounds can be used to treat musculo-skeletal inflammations. Examples of musculo-skeletal inflammations include, but are not limited to, muscle inflammations, myositis, tendon inflammations, tendinitis, ligament inflammations, cartilage inflammation, joint inflammations, synovial inflammations, carpal tunnel syndrome and bone inflammations.

According to some aspects of the disclosed method, the disclosed compounds can be used to treat idiopathic inflammations. According to some aspects of the disclosed method, the disclosed compounds can be used to treat inflammations of unknown etiology. The inflammation can be acute and/or chronic. The inflammation can be caused or exacerbated by IL-1β secretion. The IL-1β secretion can be activated by inflammasome-mediated caspase-1 activation. The inflammation can be caused or exacerbated by Vitiligo.

ii. Neurodegeneration

The condition or disease can in some aspects be a neurodegenerative disease. Thus, provided is a method of treating, preventing, or reducing the risk of developing a neurodegenerative disorder, such as Alzheimer's disease, in a subject comprising administering to the subject a therapeutically effective amount of a composition comprising the disclosed compound. Also provided is a method of treating a subject at risk for a neurodegenerative disorder, such as Alzheimer's disease, comprising administering to the subject a composition comprising the disclosed compound. As used herein, the terms “disorder” and “disease” are used interchangeably to refer to a condition in a subject.

As used herein, the term “Aβ-related disorder” or an “Aβ disorder” is a disease (e.g., Alzheimer's disease) or a condition (e.g., senile dementia) that involves an aberration or dysregulation of Aβ levels. An Aβ-related disorder includes, but is not limited to Alzheimer's disease, Down's syndrome and inclusion body myositis. Thus, the Aβ related disorder can be Alzheimer's disease. The progression of the Aβ related disorder can be slowed or reversed.

Also provided is a method for modulating amyloid-β peptide (Aβ) levels exhibited by a cell or tissue comprising contacting said cell or tissue with an amount of a composition comprising the disclosed compound, sufficient to modulate said Aβ levels.

As used herein, a cell or tissue may include, but not be limited to: an excitable cell, e.g., a sensory neuron, motomeuron, or interneuron; a glial cell; a primary culture of cells, e.g., a primary culture of neuronal or glial cells; cell(s) derived from a neuronal or glial cell line; dissociated cell(s); whole cell(s) or intact cell(s); permeabilized cell(s); a broken cell preparation; an isolated and/or purified cell preparation; a cellular extract or purified enzyme preparation; a tissue or organ, e.g., brain, brain structure, brain slice, spinal cord, spinal cord slice, central nervous system, peripheral nervous system, or nerve; tissue slices, and a whole animal. In certain embodiments, the brain structure is cerebral cortex, the hippocampus, or their anatomical and/or functional counterparts in other mammalian species. In certain embodiments, the cell or tissue is an N2a cell, a primary neuronal culture or a hippocampal tissue explant.

Also provided is a method for prevention, treatment, e.g., management, of an Aβ-related disorder, or amelioration of a symptom of an Aβ-related disorder such as Alzheimer's disease. It is understood that the methods described herein in the context of treating and/or ameliorating a symptom can also routinely be utilized as part of a prevention protocol.

Also provided is a method of treating, or ameliorating a symptom of, an Aβ-related disorder comprising administering to a subject in need of such treating or ameliorating an amount of a composition comprising the disclosed compound sufficient to reduce Aβ levels in the subject such that the Aβ-related disorder is treated or a symptom of the AP related disorder is ameliorated.

Examples of neurodegenerative disorders include Alexander disease, Alper's disease, Alzheimer disease, Amyotrophic lateral sclerosis, Ataxia telangiectasia. Batten disease (also known as Spielmeyer-Vogt-Sjogren-Batten disease), Canavan disease, Cockayne syndrome, Corticobasal degeneration, Creutzfeldt-Jakob disease, Huntington disease, Kennedy's disease, Krabbe disease, Lewy body dementia, Machado-Joseph disease, Spinocerebellar ataxia type 3, Multiple sclerosis, Multiple System Atrophy, Parkinson's disease, Pelizaeus-Merzbacher Disease, Pick's disease, Primary lateral sclerosis, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease. Transmissible spongiform encephalopathies (TSE), and Tabes dorsalis.

The condition or disease can in some aspects be Alzheimer's disease. Alzheimer's disease is a progressive neurodegenerative disorder that is characterized by the formation of senile plaques and neurofibrillary tangles containing amyloid β (Aβ) peptide. These plaques are found in limbic and association cortices of the brain. The hippocampus is part of the limbic system and plays an important role in learning and memory. In subjects with Alzheimer's disease, accumulating plaques damage the neuronal architecture in limbic areas and eventually cripple the memory process.

iii. Metabolic Disease

The condition or disease can in some aspects be a metabolic disease. Metabolic disease refers to diabetes and disorders of carbohydrate metabolism, amino acid metabolism, organic acid metabolism (organic acidurias), fatty acid oxidation and mitochondrial metabolism, porphyrin metabolism, purine or pyrimidine metabolism, steroid metabolism, mitochondrial function, peroxisomal function. Lysosomal storage disorders, Acromegaly, Addison's Disease, Cushing's Syndrome, Cystic Fibrosis, Endocrine Diseases, Human Growth Hormone related diseases, Hyperparathyroidism, Multiple Endocrine Neoplasia Type 1, Prolactinoma, Turner Syndrome.

Thus, the condition or disease can in some aspects be diabetes. The World Health Organization recognizes three main forms of diabetes: type 1, type 2, and gestational diabetes (occurring during pregnancy), which have similar signs, symptoms, and consequences, but different causes and population distributions. Type 1 is usually due to autoimmune destruction of the pancreatic beta cells which produce insulin. Type 2 is characterized by tissue-wide insulin resistance and varies widely; it sometimes progresses to loss of beta cell function. Gestational diabetes is similar to type 2 diabetes, in that it involves insulin resistance. The hormones of pregnancy cause insulin resistance in those women genetically predisposed to developing this condition. Types 1 and 2 are incurable chronic conditions, but have been treatable since insulin became medically available in 1921. Gestational diabetes typically resolves with delivery. Thus, in some aspects of the disclosed method, the subject has been diagnosed with or is at risk for type 1 diabetes mellitus.

Thus, provided is a method of treating or preventing diabetes in a subject, comprising administering to the subject a composition comprising the disclosed compound.

iv. Stress

The NOD proteins are also involved in the AP-1 pathway (including the stress kinase pathway). Thus, the disclosed modulators and methods can be used to modulate these pathways, their activities, and their effects. For example, subjects that have been or may be subjected to stress can be treated according to the disclosed methods. The NOD modulators would serve to affect the stress signaling pathway. Many sources of stress invoke the stress signaling pathway and so any or a combination of such stresses can be treated using the disclosed NOD modulators and the disclosed methods.

v. Interferon Response Factor Related Disorders

The NOD proteins are also involved in the Interferon Response Factor (IRE) pathways. Thus, the disclosed modulators and methods can be used to modulate these pathways, their activities, and their effects. For example, subjects that have been or may be subjected to pathogens or that suffer disease conditions implicating interferon can be treated according to the disclosed methods. The NOD modulators would serve to affect the Interferon Response Factor pathways. Many sources invoke the Interferon Response Factor pathways and so any or a combination of such stresses can be treated using the disclosed NOD modulators and the disclosed methods.

### Kits

The compositions and other materials described above as well as other materials can be packaged together in any suitable combination as a kit useful for performing, or aiding in the performance of, the disclosed methods. It is useful if the kit components in a given kit are designed and adapted for use together in the disclosed method. For example disclosed are kits suitable for identifying potential modulators of NOD1, NOD2, or both, the kit comprising constructs and/or cells. For example disclosed are kits suitable for treating diseases modulated by NOD1, NOD2, or both.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Materials and Methods

Reagents: Ala-γ-D-Glu-DAP (γtri-DAP) was obtained from Anaspec and Invivogen. Muramyl dipeptide (L-isoform), Poly(dA:dT), Poly(LC) and TLR agonist panel (Human TLR1-9 Agonist kit) were purchased from Invivogen. Recombinant human TNF-α was acquired from R&D Systems. Phorbol-12-myristate-13-acetate (PMA), ionomycin, ATP and the proteosome inhibitor MG-132 were from Calbiochem (EMD Chemicals). Doxorubicin and monosodium urate (MSU) were obtained from Sigma-Aldrich. Recombinant human BAFF soluble protein (catalog no. ALX-522-025-0010) was from Alexis Biochemicals (Enzo Life Sciences). Lipopolysaccharides (LPS) were purchased from Alexis Biochemicals (Enzo Life Sciences) and Sigma-Aldrich. Wild type serovar Enteritidis LK5 was used for infection experiments.

Antibodies: Monoclonal RIP2/RICK (catalog no. 612348) and mouse anti-human SGT-1 (catalog no. 612104) antibodies were from BD Transduction Laboratories. Rabbit and rat anti-NOD1 antibodies were respectively purchased from Imgenex (catalog no. IMG-5739) and obtained from University of Cologne, Germany. Mouse anti-FLAGM2 (catalog no. F3165), monoclonal anti-β-actin (catalog no. A5441) and anti-α-tubulin (catalog no. T9026) antibodies were from Sigma-Aldrich. Monoclonal anti-c-myc (9E10) antibody was supplied by Roche. Anti-ubiquitin P4DI mouse antibody (catalog no. 3936), rabbit anti-PARP (catalog no. 9542) and pan-cadherin (catalog no. 4068) antibodies were from Cell Signaling Technology. Lysine-specific rabbit antibodies against Lys48 (catalog no. 05-1307) and Lys63 (catalog no. 05-1308) ubiquitin were from Millipore.

Cell culture: Human embryonic kidney (HEK) 293T cells and MCF-7 breast cancer cells were maintained in Dulbecco modified Eagle medium (DMEM) (CellGro, Mediatech) supplemented with 10% heat-inactivated fetal bovine serum (FBS) and 1% antibiotic/antimycotic solution (Omega Scientific) at 37° C. in an atmosphere of 10% and 5% CO₂, respectively. THP-1 monocytic cells and pre-B acute lymphocytic leukemia (ALL) 697 cells were cultured in RPMI 1640 medium (CellGro, Mediatech) with the same supplements, at 37° C. in an atmosphere of 5% CO₂. HCT-116 colon carcinoma cells were cultured in McCoy's 5A medium (Invitrogen) with the same supplements, at 37° C. in an atmosphere of 5% CO₂. RAW 264.7 cells (Mouse leukaemic monocyte macrophage cell line) were maintained in DMEM supplemented with 10% heat-inactivated FBS and 1% antibiotic/antimycotic solution at 37° C. in an atmosphere of 5% CO₂. For isolation and culturing of human dendritic cells, CD14+ monocytes were isolated as the adherent fraction of human peripheral blood mononuclear cells from healthy donors after incubation for 1 hr in RPMI 1640 (BioWhittaker, Inc.) supplemented with 10% fetal calf serum and 100 U/ml penicillin-streptomycin (Bristol-Myers Squibb) at 37° C. After extensive washing, adherent monocytes were differentiated into monocyte derived dendritic cell by culture in complete medium with the addition of 10 ng/ml recombinant IL-4 (Peprotech) and 50 ng/ml recombinant granulocyte macrophage colony-stimulating factor (GM-CSF) (Peprotech).

Flow Cytometry: After 6-day differentiation, DC were washed and either left unstimulated or stimulated with 5 μg/ml γ-tri-DAP or with 100 ng/ml LPS, in the absence or presence of CID-1088438 (15 μM), for 24 hr before FACS analysis. CID-1088438 was added to the culture 30 min prior to stimulation. DC activation was checked by flow cytometry (BD FACSAria™ II, BD Biosciences) to determine the expression of CD83, HLA-DR and CD 86 using fluorochrome-conjugated monoclonal antibodies (BD Biosciences).

Lentiviral production and purification: Lentivirus Production. Vesicular stomatitis virusGenvelope protein-pseudotyped lentiviruses were produced in HEK293T cells and purified as described (Tiscornia et al. (2003). Naldini et al. (1996), Pfeifer et al. (2001)). In other examples, full-length human cDNA sequences encoding NOD1 and NOD2 were modified by PCR (Advantage 2 kit. Clontech) to encode 6×His and flag-epitopes tags at their N-terminal, and respectively subcloned into XbaI and XhoI sites of a CMV-driven lentiviral construct pCSC-SP-PW. For functional assays, a third generation of lentiviral vector was also utilized to introduce a 5×κB-responsive firefly luciferase cassette (Tergaonkar et al., 2003), allowing the expression of the reporter gene under the control of five tandem HIV NF-κB response elements (obtained from Salk Institute, CA, USA). Vesicular stomatitis virus G envelope protein-pseudotyped lentiviruses were prepared and purified as described (Naldini et al., 1996; Pfeifer et al., 2002; Pfeifer and Verma, 2001). Vector concentrations were estimated according to biological titer provided by GFP expressing lentiviruses (control).

Gene reporter assays: HEK293T or 697 cells containing integrated 5×NFκB-driven luciferase reporter gene were seeded into 96-well plates at 10⁴ to 10⁵ cells per well, and treated with respective inducers for 16 to 24 hours. Luciferase activity was measured as suggested by manufacturer's protocol (Steady-Glo™ Luciferase Assay System, Promega), using a FlexStation 3 Microplate Reader (Molecular Devices). Same experimental procedures were applied for HEK293T cells stably expressing luciferase reporter gene driven by interferon responsive elements (ISRE). THP.1-Blue™ monocytic cells (Invivogen), stably containing NF-κB-driven secreted alkaline phosphatase reporter (SEAP), were plated and further treated as indicated. Stimulation was accessed colorimetrically measuring SEAP secreted into culture supernatants (QUANTI-Blue'm, Invivogen), using SpectraMax 190 plate reader (Molecular Devices). Wild-type and XIAP deficient HCT116 cells were seeded at a density of 2×10⁴ cells per well in 96-well plates. The next day, cells were transfected with 50 ng pNF-κB-LUC (Clontech) and 5 ng Renilla luciferase gene driven by a constitutive TK promoter (pRL-TK; Promega) along with indicated plasmids. After 24 h of transfection in some experiments, cells were stimulated with various agents for 24 h or directly lysed and luciferase activities were assayed using the Dual Luciferase kit (Promega). The results for firefly luciferase activity were normalized to renilla luciferase activity. In experiments with wild-type or transduced HEK 293T cells with stably integrated 5×NFκB-mediated luciferase reporter gene, cells were seeded into 96-well plates at 10⁴ to 10⁵ cells per well, and treated with respective inducers for 16 to 24 h. Luciferase activity was measured as suggested by manufacturer's protocol (BriteLite reagent, Perkin-Elmer). The mean results were obtained from triplicates.

High throughput screening (HTS): HEK293T cells containing stably integrated NF-κB luciferase (Luc) reporter were harvested as described, with the exception that after removing supernatant, cell pellets were washed once in Dulbecco's phosphate buffered saline and further re-suspended in assay medium supplemented with 0.62% DMSO to a density of 2×106 cell per ml. Cell suspension was then plated into white 1536 well tissue culture treated assay plates (model 3727, Corning) at 3 μl per well (6×10³ cells per well) using a Multidrop Combi (Thermo Scientific). Plates were centrifuged for 5 minutes at 500 rpm on an Eppendorf 5810 centrifuge. Ten nanoliter (n1) test compounds solvated to 2 mM in DMSO and stored in 1536 well Cyclic Olefin Co-polymer (COC) source plates (model 3730, Corning) were delivered to columns 5-48 and 10 n1 DMSO control to columns 1-4 using a HighRes Biosolutions pin-tool equipped with V&P Scientific pins. Plates were then incubated for 60 minutes at room temperature. Next, 2 μl of γ-tri-DAP at 1.874 μg/ml (Anaspec, Freemont, Calif.) in assay medium (for a final concentration of 0.75 μg/ml γ-tri-DAP) was added to columns 3-48 and 2 μl assay medium added to columns 1-2 using a Multidrop Combi. Final concentration of test compounds in assay was 4 μM. Final DMSO concentration was 1%. Plates were centrifuged for 30 seconds at 1000 rpm (200×G) on an Eppendorf 5810 centrifuge and incubated 16 hours at 37° C. in 5% CO₂. After incubation, plates were removed from incubator and allowed to equilibrate to room temperature (10-15 minutes). Three microliters of Steady-Glo® luciferase assay detection reagent (Promega) was added to entire plate using Multidrop Combi and immediately centrifuged for 30 seconds on a VSpin™ integrated microplate centrifuge at 1500 rpm (Velocity11/Agilent Technologies) and incubated for 20 minutes at room temperature. Luminescence was read on a Viewlux microplate imager (PerkinElmer). For the purpose of a single concentration primary NOD2 counter-screen, a stable line based on the HEK293T-NF-κB-Luc constitutively over-expressing NOD2 was developed as a surrogate. Experimental procedures were followed but in the absence of any inducer.

ELISA: For IL-8 ELISA, MCF7 cells stably expressing His6-FLAG-tagged NOD1 or NOD2 were produced by lentivirus infection. Cells were cultured up to 90% confluency. Assays were initiated using 5,000 cells per well (in 100 μl volume) of a 96-well transparent culture plate. After cell attachment, the culture medium was replaced with Phenol Red-free medium containing 1 μg/ml γ-Tri-DAP plus 1.5 cycloheximide (100 microliters final) and incubated for 16 to 20 hours at 37° C. in an atmosphere of 5% CO₂. Supernatants were collected into a new 96-well plate and stored at −80° C. IL-8 levels were measured using a human IL-8 ELISA (BD OptEIA™ Human IL-8 ELISA Set, BD Biosciences). For IL-1β ELISA, assays were initiated using 50,000 RAW264.7 cells per well (in 100 μl of Opti-Mem medium, Invitrogen) of a 96-well transparent culture plate. Cell treatments were performed as indicated. IL-1β levels were measured using mouse IL-1 beta ELISA Ready-Set-Go!™ Kit (E-Biosciences).

Protein analysis: For immunoprecipitation (IP) assays, cells were lysed with IP buffer (20 mM Tris-HCl [pH 8.0], 250 mM NaCl, 0.05% NP-40, 3 mM EDTA, 3 n3M EGTA) supplemented with 1 mM dithiothreitol (DTT) and protease inhibitor cocktail (Roche). Equal amounts of clarified protein lysates (1-2 mg) were respectively incubated with 1 μl of rabbit anti-NOD1 antibody (Imgenex) plus protein A-Sepharose CL-4B (Amersham) or 20 μl of anti-FLAG™ M2 affinity gel (Sigma), in 1 ml of IP buffer for 12 to 16 hours at 4° C. For Ni/NTA pull-down, cells were lysed with phosphate lysis buffer (50 mM sodium phosphate [pH 8.0], 1.50 mM NaCl, 5 mM imidazole, 0.25% Triton X-100) supplemented with 5 mM 2-mercaptoethanol and EDTA-free protease inhibitor cocktail (Roche). Equal amounts of clarified protein lysates (1-2 mg) were incubated with 30-40 μl of Ni-NTA agarose beads (Qiagen) in 1 ml of buffer for 4 to 12 hours at 4° C. Immunoprecipitates and Ni/NTA-bound proteins were then washed three times with respective lysis buffer and eluted with 1% acetic acid. After vacuum drying (Vacufuge™, Eppendorf), pellets were resuspended with 1× sample buffer (50 mM Tris [pH 6.8], 2% SDS, 10% glycerol, 0.01% bromophenol blue, 5% 2-mercaptoethanol) and analyzed by SDS-PAGE. For subcellular fractionation, cytosolic fractions were obtained by extensive resuspension of cell pellets into buffer A (10 mM Hepes 7.5, 1.5 mM MgCl₂, 10 mM KCl, 0.5 mM DTT and protease inhibitors). Remaining pellet was extensively resuspended into buffer C (20 mM Hepes 7.5, 25% glycerol, 450 mM NaCl, 1.5 mM MgCl₂, 0.2 n3M EDTA and protease inhibitors) to isolate nuclear sub-fraction, followed by resuspension of final pellet (cell membrane sub-fraction) into RIPA extraction buffer (Thermo Scientific). Immunoblotting was performed using respective antibodies. Total lysates (50-60 μg) and subcellular fractions (10 μg) were also immunoblotted after protein quantification (Bio-Rad Protein Assay).

In another set of experiments, for immunoprecipitation (IP), cells were lysed in IP buffer [20 mM iris pH 7.5, 135 mM NaCl, 1 mM EDTA or 1 mM EGTA (for binding assays involving NODS), 0.5% Nonidet P-40, 10% glycerol, 10 mM NaF, 1 mM DTT, 2 mM Na₃VO₄, 20 μM leupeptin, 1 mM PMSF, 20 mM N-ethylmaleimide, 0.5 μM iodoacetic acid, 1× protease inhibitor mix (Roche Applied Science)]. Clarified protein lysates (1-2 mg) were incubated with 2 μg monoclonal anti-FLAG antibody (Sigma Aldirch), 2 μg monoclonal anti-GFP antibody (Santa Cruz Biotechnology), or 8 μg monoclonal anti-RIP2 antibody (Alexis Biochemicals) prelinked to 25-50 μg recombinant protein G Sepharose (Invitrogen) at 4° C. For GST pulldown experiments, recombinant GST-fusion proteins were preincubated with 25 μg glutathione-Sepharose 4B (GE Healthcare) at 4° C. and mild rotation for 1 h. Beads were centrifuged at 3,400 rpm for 5 min, supernatants removed and incubated with 1 mg cell lysates in IP buffer at 4° C. with rotation. After incubation overnight bound immune complexes were washed four times in IP buffer, boiled in 2× Laemmli buffer and analyzed by SDS/PAGE and immunoblotted using various antibodies as specifically indicated. Lysates (50 μg) were also directly analyzed by immunoblotting after normalization for total protein content.

Protein expression and purification: NOD1 protein was purified from 293 Freestyle™ cells (Invitrogen) stably expressing 6×His-FLAG-NOD1 transgene. Purification was performed using the FLAG M Purification Kit (Sigma). Briefly, cells were grown in final volume of 2 liters, under agitation at 37° C. in an atmosphere of 8% CO₂. Cells lysates were produced by sonication and further cleared by centrifugation of 20,000×g for 30 minutes. Cleared lysates were incubated with anti-Flag beads in batch mode, then beads were loaded on a column and a wash step of 500×CV (column volume) was performed, followed by elution using 0.1 M glycine pH 3.5. Protein concentration was determined using BCA Protein Assay (Thermo Scientific).

Fluorescent polarization assay (FPA): FPAs were performed as described previously using various purified recombinant NOD1, NLRP1ΔLRR, or HSP70 ATP binding domain proteins and FITC-conjugated ATP (Fluorescein-12-ATP, PerkinElmer) (Zhai et al., 2006). Briefly, recombinant proteins were incubated in 384-well black round-bottom plates with 10 nM of FITC-ATP in a total volume of 20 μl in the dark. Fluorescence polarization was measured using a LJL Analyst HT plate reader (Molecular Devices) in PBS, 0.005% Tween-20. IC₅₀ determinations were performed using GraphPad Prism software.

NMR spectroscopy: ¹H-NMR experiments were performed at 25° C. on a 500-MHz Bruker Avance spectrometer (Bruker, Madison, Wis.) equipped with a 5-mm TXI probe. Compounds were dissolved in fully deuterated DMSO (d6-methylsulfoxide; Sigma-Aldrich) to a concentration of 10 mM. 1H NMR reference spectra were collected at a final concentration of 50 μM in 50 mM Tris-HCl, pH7.5, 150 mM NaCl buffer with or without 5 μM of 6×His-Flag-NOD1, 6×His-Bcl-XL, or 6×His-Bid proteins. All 1H-NMR spectra were obtained with the carrier position set to the water peak signal using WATERGATE™. NMR data were processed and analyzed with xwinplot.

RNA analysis: Total RNA was extracted using RNeasy™ Plus Mini kit (Qiagen). After isolation, 1-5 μg total RNA was reverse-transcribed, in the presence of oligo (dT) primer, according to the manufacturer (SuperScript First-Strand Synthesis, Invitrogen). First strand cDNA was diluted and analyzed in triplicates with gene-specific primers by realtime PCR, using a Stratagene Mx3000p sequence detection system with SYBR Green PCR master mix (Applied Biosystems). The gene expression (fold induction) was normalized with the respective levels of beta-actin or cyclophilin (CPH) expression. For TaqMan™-based quantitative PCR, assays were performed using TaqMan™ Gene Expression Master Mix with validated primers and probes (TNF: Hs00174128_ml, IL1B: Hs01555410_ml, IL6: Hs00985639_ml, NOD1: Hs00196075_ml, ACTB: hs999999903_ml) from Applied Biosystems.

Expression Plasmids: Plasmids encoding human FLAG-XIAP, FLAG-SIP, GFPRIP2WT, GFP-RIP2ΔCARD, GFP-RIP2Δkinase domain, human Myc-NOD1, Myc-NOD1ΔCARD, Myc-NOD2, Myc-NOD2ΔCARD1 and Myc-NOD2ΔCARDs have been recently described (Matsuzawa et al. (2001), Lu et al. (2007), Krieg et al. (2009)). XIAP-targeting shRNA vector was created by designing a 83-mer oligonucleotide containing an XbaI site at the 5′ end and sense and antisense shRNA strands separated by a short spacer, plus a partial sequence of the H1-RNA promoter at the 3′ end. Standard PCR procedures (Advantage 2 PCR kit, Clontech) were performed by using specific shRNA oligonucleotides and T3 primer plus pSuper-like plasmid (Tiscornia et al. (2003)) as a template to provide H1-mediated shRNA cassettes with an additional XbaI site at the 3′ end. The following shRNA oligonucleotides were used: 5′-CTGTCTAGACAAAAAGTGGTAGTCCTGTTTCAGCTCTCTTGAA GCTGAAACAGGACTACCACGGGGATCTGTGGTCTCATACA-3′ (SEQ ID NO: 1) for XIAP, and 5′-CTGTCTAGACAAAAAGCTTCTGCTCGCCAATAAATCTCTTGAATTTA TTGGCGAGCAGAAGCGGGGATCTGTCiGTCTCATACA-3′ (SEQ ID NO as scrambled control. PCR products were purified (Qiagen), digested with XbaI, and cloned into the 3′ LTR NheI site of a CMV-GFP lentiviral vector as described (Tiscornia et al. (2003)).

### Example 1

### High Throughput Screening Assays for NOD1 and NOD2 Inhibitors

The cell-based HTS assays utilize NF-κB-mediated luciferase reporter gene activity as a measure of NOD1 and NOD2 modulation. A secondary assay to confirm compound selectivity towards NOD activity, by measuring secretion into culture supernatants of interleukin-8 (IL-8), an endogenous NF-κB target gene is also performed. When combined with insights provided by cheminformatics analysis, and a variety of additional downstream assays provided by the assay provider for deconvoluting hits, thus, the assays can identify candidate compounds for NOD1 and NOD2 modulation which can be further optimized using medicinal chemistry.

One assay is a cell-based, Luciferase reporter gene assay used in high throughput screening (HTS) to identify chemical inhibitors of NOD-dependent NF-κB activation. The primary HTS assays use NOD1 or NOD2 to drive NF-κB-responsive luciferase reporter genes. Various downstream counter-screens and secondary assays can be employed to further characterize the selectivity of the hits, setting the stage for subsequent compound structure/activity relation (SAR) studies and laying a foundation for chemical probe optimization.

Lentiviral vectors. Third-generation of lentiviral vectors (Pfeifer, A. and Verma, I. M. Annu Rev Genomics Hum Genet, 2: 177-211, 2001; Pfeifer, A. et al., PNAS, 99: 2140-2145, 2002) were designed to introduce a 5×κB-responsive firefly luciferase cassette, allowing the expression of the reporter gene under the control of five tandem HIV NF-κB response elements, and to also express 6×His-FLAG-NOD1 or -NOD2, in the cells of interest. This cell line was chosen for the assay based on the current literature supporting the use of human embryonic kidney (HEK) 293T cells to perform biochemical studies of NLR proteins.

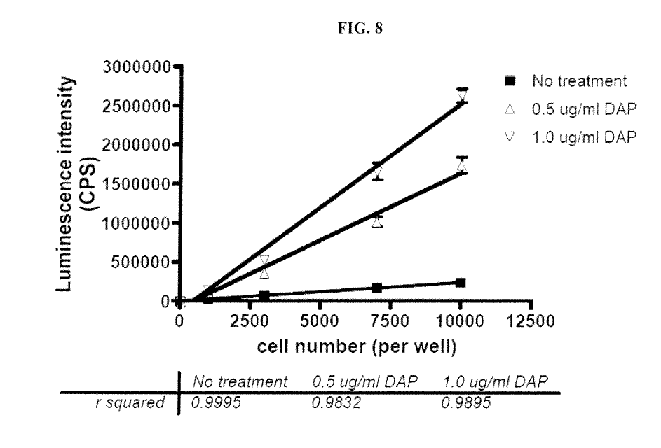

NOD1 cell-based luciferase assay. Various numbers of transduced 5×κB-Luciferase 293T cells were seeded into 384-well plates, to determine over what range of cell densities the luciferase signal from the NF-κB reporter gene is linear, and two different concentrations of the NOD1-specific inducer Ala-γ-Glu-diaminopimelic acid (γ-tri-DAP) (0.5 and 1 μg/ml) were compared. An r²>0.98 value was obtained for all experimental conditions, ranging from 10³ to 10⁴ cells per well, demonstrating linearity of the signal over this range of cell densities, without reaching a plateau in the assay (see FIG. 8).

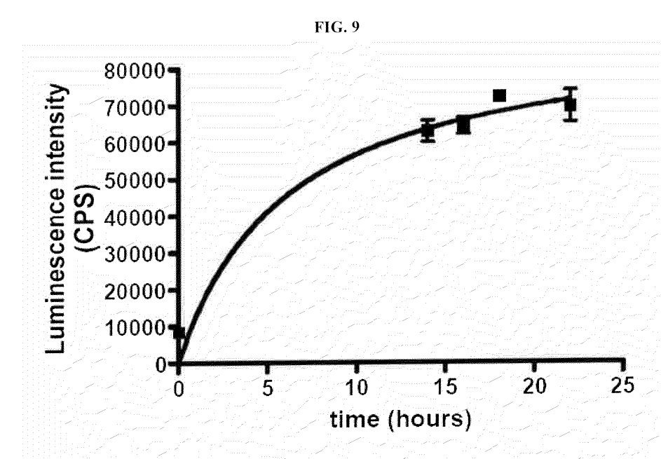

The optimal time to measure luciferase activity was determined after induction, a time-course assay was performed using fixed amounts of the NOD1-activating ligand γ-tri-DAP. Increased luciferase signal was clearly evident after 14 hours of treatment, reaching its highest value at 18 hours post-treatment (see FIG. 9). This assay was repeated to confirm that luciferase signal was higher after 20 hours of incubation (data not shown). Though more extensive time-course studies should be performed, these data show that the assay is stable at least within the assay windows of 14-22 hrs, an 8 hr interval.

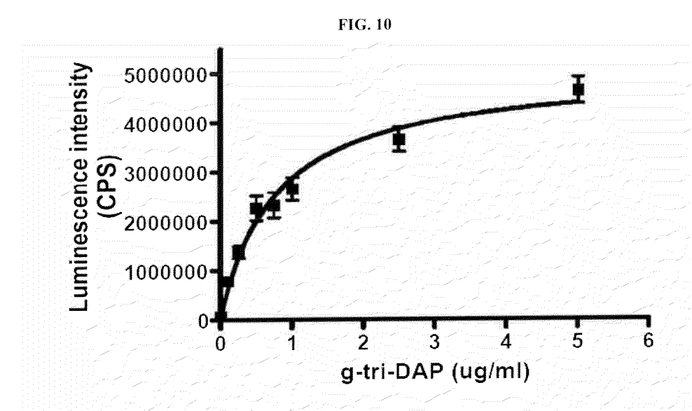

The optimal concentration of γ-tri-DAP for the assay was determined by titrating various concentrations of the NOD1-activating ligand into the luciferase reporter cell lines, using 10⁴ cells per well. Increasing concentrations of γ-tri-DAP resulted in dose-dependent induction of the NF-κB-driven luciferase reporter gene, which was linear at concentrations <1 μg/mL, approaching saturation at >3 μg/ml γ-tri-DAP (see FIG. 10). Half-maximal κB-dependent luciferase activity was achieved with ˜0.75 μg/ml γ-tri-DAP (highest assay sensitivity).

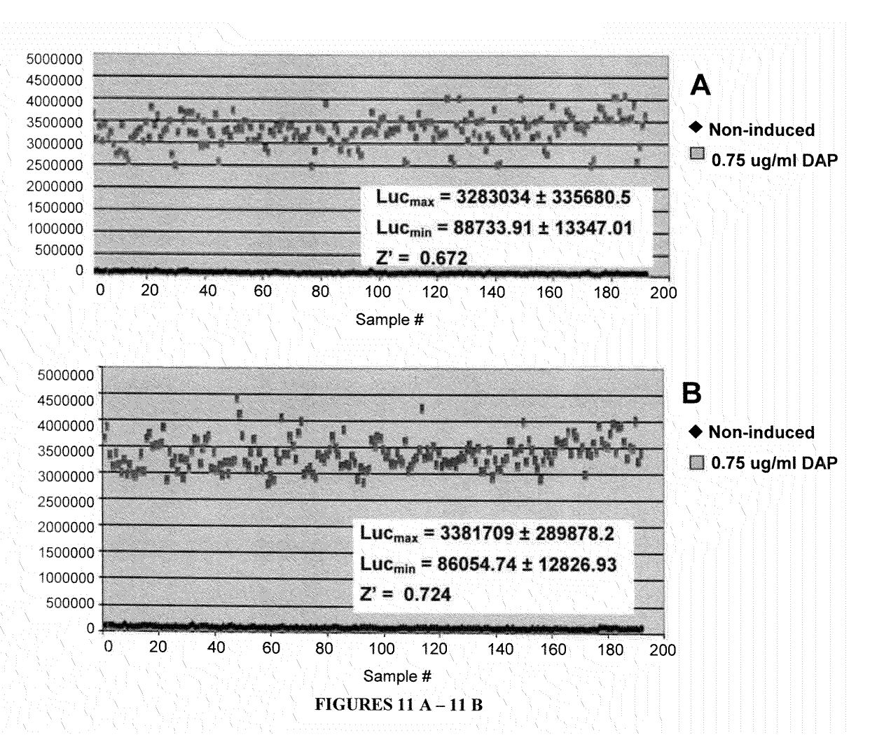

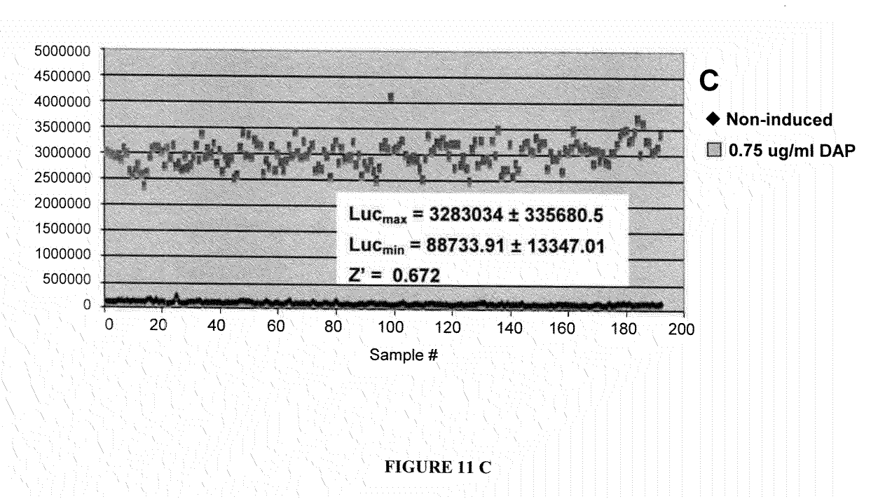

Next, the Z′ factor of the assay was determined to evaluate its suitability and reproducibility for HTS. The assay was conducted in 384 well-format using LJL Analyst at different days and times (see FIG. 11). Despite of the decrease of luciferase signal after longer periods of incubation with substrate (20-30% decrease after 30 minutes of incubation), the signal to noise ratio was essentially constant (data not shown). Luminescence values were plotted versus well number. The Z′ values were consistently in the range of 0.67 to 0.73 (see FIG. 11).

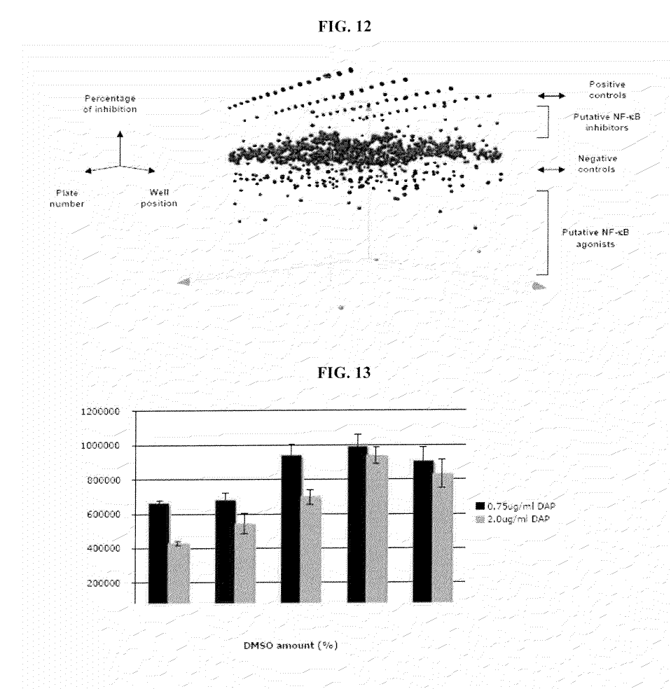

Based on the assay configuration as described above, a screen of a small chemical library (LOPAC, Sigma-Aldrich) was performed to test the performance of the HTS in 384-well format. The screening data are plotted in FIG. 12. The first and the last two lanes of each plate were used as negative (0% inhibition, induction only) and positive (100% inhibition, no induction) controls, respectively. A DMSO-induced shift in the data was present, with a decrease in luciferase signal for the entire data set of ˜20-25%, thus accounting for the difference between the negative control and compound-treated wells. Under our experimental conditions, increasing amounts of DMSO (up to 1%) augment the luminescence signal after γ-tri-DAP treatment (see FIG. 13). This variation can be corrected by adjusting the concentration to a range of 0.5% to 1% DMSO, since changes are less noticeable (FIG. 13). Nevertheless, this DMSO-induced variation did not affect the HTS performance.

From a screen of 1280 chemical compounds, 29 hits was observed (cut-off of 50% inhibition), including known NF-κB inhibitors such as sanguinarine, parthenolide and N-tosyl-L-phenylalanine chloromethyl ketone (TPCK) (see FIG. 12, top). Three hits were also identified (cut-off of 90% induction) as NF-κB agonists: the anthracycline idarubicin, phorbol 12-myristate 13-acetate (PMA), and the glutamate receptor agonist 1-aminocyclopropanecarboxylate (ACPC) (FIG. 12, bottom). Taken together, the performance of the NOD1 cell-based assay was suitable for HTS screening.

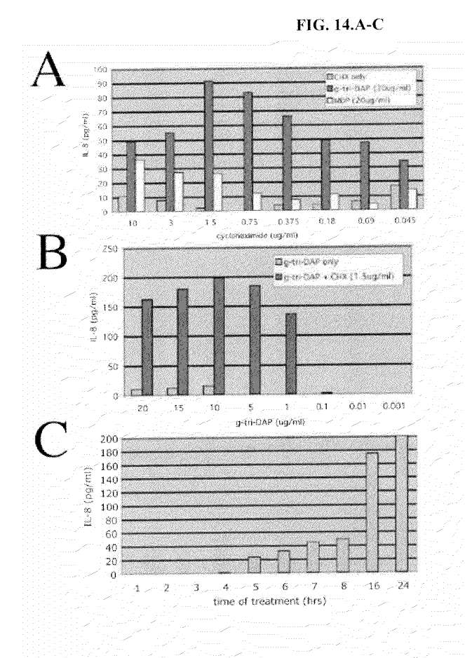

NOD1 secondary assay. Interleukin-8 (IL-8) is an important mediator of the immune reaction and a major chemokine involved in inflammatory responses. Recent studies have indicated that γ-tri-DAP induction of human breast cancer epithelial cell lines MCF-7 over-expressing NOD1, combined with small doses of cycloheximide (CHX), specifically induces IL-8 production and release (da Silva Correia, et al., Cell Death Differ, 14: 830-839, 2007; da Silva Correia, et al., PNAS, 103: 1840-1845, 2006). Therefore, a biochemical-based assay to measure IL-8 levels by ELISA was devised (BD Biosciences), using stably transfected MCF-7 cells over-expressing NOD1. Using 96-well plates, 5000 cells per well were seeded and preliminary assays were performed to optimize the cycloheximide and γ-Tri-DAP concentrations, and the time of drug treatment (see FIG. 14A to C). The data indicates that the use of 1 to 5 μg/ml γ-tri-DAP plus 1.5 μg/ml cycloheximide for 24 hours induces robust IL-8 production. This secondary assay is used to validate the hits resulting from the primary luciferase-based assay, thus using an orthogonal read-out relying on an endogenous NF-κB target gene (IL-8 production) and a different cell line (MCF-7 vs. 293T), thereby avoiding further work on compounds that show activity only in reporter gene assays or only in 293T cells.

Development of NOD2 cell-based luciferase assay. The NF-κB-luciferase 293T cells described above were stably infected with a lentivirus to deliver cytomegalovirus (CMV) promoter-driven expression of 6×His-FLAG-NOD2. Since these viruses were simultaneously able to deliver IRES-mediated EGFP, transduced cells were sorted by flow cytometry (FACSDiVa), resulting in a population of NOD2-over-expressing cells. An NF-κB-luciferase-based assay was devised that could be performed independently of any NOD2-specific inducer; relying on the observation that over-expression of NOD2 is sufficient to lead to its activation. Similar procedures were also performed using 6×His-FLAG-NOD1 to provide NOD1-over-expressing cells (data not shown). Using experimental conditions as described for NOD1 above, the performance of the HTS assay was tested in 384 well plate format, comparing EGFP over-expressing cells (positive control, 100% inhibition) and NOD2-expressing cells (negative control, 0% inhibition). The Z′ factor was determined to be 0.71, which is sufficiently robust for HTS (see FIG. 15). A negative control for these ISIS assays can also be established, as an alternative to using cells lacking NOD1 or NOD2, so that a separate cell line need not be prepared and separately dispensed into some wells. In this regard, it was shown that compounds inhibiting IKK suppress NF-κB-luciferase reporter gene activity in the cell-based assays, providing one option. It was also confirmed from recent reports that Hsp90 is required for stabilization of NOD1 (Hahn, J. S. FEBS Lett, 579: 4513-4519, 2005; Goetz, M. P. et al., Ann Oncol, 14: 1169-1176, 2003), by showing that Hsp90 inhibiting compounds geldanomycin and 17-AGG also suppress NOD-driven NF-κB reporter gene activity (not shown). Thus, either IKK or Hsp90 inhibitors may be acceptable alternatives that simplify the HTS procedures.

A screen using the LOPAC chemical library was performed to test the performance of the NOD2 FITS in 384-well format (see FIG. 16). From a screen of 1280 chemical compounds, 6 inhibitory hits were obtained (cut-off of 50% inhibition), where five of them were consistently present among the NOD1 hits. Interestingly, 11 agonists were identified (cut-off of 50% induction) that included known NF-κB inducers, such as brefeldin A, etoposide, idarubicin and colchicine. These are all cytotoxic anti-cancer drugs, which are known to induce NF-κB activity. Thus, the performance of the NOD2 cell-based assay was also suitable for HTS screening.

Luciferase-based primary assays. HEK-293T cells stably transduced with the lentiviral vectors expressing 5×B-Luc were cultured in 15-cm dishes at 37° C., 10% CO₂ and 90% relative humidity. The growth media consisted of Dulbecco's Modified Eagle's Media (DMEM) supplemented with 10% v/v heat inactivated fetal bovine serum and 1% v/v penicillin-streptomycin mix. Prior to the assay, cells were suspended to a concentration of 263,000 cells per milliliter in phenol red free DMEM without serum. The assay began by dispensing 38 microliters of cell suspension to each well (i.e. 10,000 cells/well) of a white solid-bottom 384-well plate, pre-loaded with 2 microliters of chemical compounds at 100 in 10% DMSO. Plates were pre-incubated at room temperature for one hour. For NOD1 assays, ten microliters of γ-tri-DAP were then dispensed to each plate well at 0.75 μg/ml final concentration, and plates were incubated for 16 hours at 37° C., 10% CO₂ and 90% relative humidity. Positive controls (100% inhibition, medium only) were loaded onto first two rows, and negative controls (0% inhibition, inducer only) were loaded on the last two rows of each plate, containing 2 microliters of 10% DMSO each. For NOD2 assays, no treatment with inducer was performed but followed the same incubation period. Positive controls (100% inhibition, EGFP-expressing cells) were loaded into the first two rows, and negative controls (0% inhibition, NOD2-expressing cells) were loaded into the last two rows of each plate, containing 2 microliters of 10% DMSO each. After incubation, plates were equilibrated to room temperature for 15 minutes. A luciferase assay was then performed by adding 20 microliters per well of the BriteLite™ Assay System reagent (Perkin Elmer). After a ten minute incubation at room temperature, light emission was measured with the Analyst™ Microplate reader (LJL Biosystems). Alternatively, compounds obtained as potential hits on the preliminary NOD1 and NOD2 screens will be confirmed and used as positive control for HTS.

ELISA-based secondary assays. NOD1-over-expressing MCF7 cells were cultured in 10-cm dishes at 37′C, 5% CO₂ and 95% relative humidity. The growth media consisted of RPMI 1640 supplemented with 10% v/v heat inactivated fetal bovine serum, 1% v/v penicillin-streptomycin mix and 100 μg/ml G-418. Assays were initiated by dispensing, 100 microliters of cell suspension to each well (i.e. 5,000 cells/well) of a 96-well transparent plate. After cell attachment, the culture medium was replaced with Phenol Red-free medium containing 1 μg/ml γ-Tri-DAP plus 1.5 μg/ml cycloheximide (100 microliters final) and incubated for sixteen to twenty hours at 37° C., 5% CO₂ and 95% relative humidity. Supernatants were collected into a new 96-well plate and stored at −80° C. IL-8 levels were measured using a protocol for Human IL-8 ELISA followed as suggested by the manufacturer (BD Biosciences, cat #555244).

Secondary Assays. A panel of secondary assays can be used to determine the selectivity of compounds and to exclude false-positives. These secondary assays are based on the knowledge that 7 different NF-κB activation pathways have been identified to date, only one of which is activated by NLR-family proteins, such as NOD1 and NOD2. The seven NF-κB pathways are; (1) NLR-driven (e.g. NOD1, NOD2); (2) PKC-driven (phorbol esters such as PMA and T-cell/B-cell antigen receptors); (3) TNF-driven (TNF and other members of this cytokine pathway that stimulate the “classical” NF-κB pathway); (4) Alternative NF-κB activation pathway (which is triggered by selected members of the TNF-family of receptors, including Lymphotoxin-beta Receptor, BAFF-Receptor, and CD40); (5) TLR-driven pathway, which is stimulated by LPS (TLR4 agonist) and CpG (TLR9 agonist); (6) Rig/Helicard pathway, which is stimulated by single-strand RNA molecules; and (7) DNA-damage pathway, which involves the p53-inducible NF-κB-activator, PIDD. Compound validation can be performed as outlined below:

1. Repeat testing of hits using the same NF-κB-reporter gene assays.

2. Perform luciferase-based ATP content assay to determine cytotoxicity.

3. Perform luciferase assay to exclude luciferase inhibitors (may be determined by PubChem database analysis because several luciferase inhibitors have already been identified within the NIH library).

4. Perform LC-MS quality control analysis of cherry-picked hits

5. Perform IC₅₀ dose-response testing using the same primary reporter gene assay, selecting hits with appropriate concentration-dependent behavior and IC<20 μM.

6. Perform counter-screen using NOD1 or NOD2-transfected MCF-7 cells secreting IL-8, an endogenous NF-κB target gene, thus eliminating compounds that only work in 293T and that only suppress in a reporter gene assay, determining IC₅₀. (note that γ-Tri-DAP and MDP ligands are used to activate NOD1 and NOD2, respectively, in MCF-7 cells).

7. Perform NF-κB pathway selectivity screens to eliminate compounds that suppress downstream components of the NF-κB induction machinery or that act on other pathways:
(a) 293 cells containing stably integrated NF-κB luciferase reporter gene, stimulated with PMA (to stimulate the PKC-driven pathway); Doxorubicin (to stimulate the DNA damage-driven pathway); TNF (to stimulate the classical NF-κB pathway); or Lymphotoxin-beta (to stimulate the alternative NF-κB pathway).
(b) MCF-7 cells stimulated with the same agents (PMA, Doxorubicin, TNF, LT-beta), measuring IL-8 production instead of NF-κB luciferase reporter gene.
(c) THP.1 monocytic cell line, differentiated with TPA to produce macrophages, then stimulated with either LPS or CpG (TLR agonists), using IL-6 secretion as the read-out (alternatively, commercially available THP.1 cells containing stably integrated NF-κB-driven β-galactosidase reporter gene are used in our laboratory).

8. Perform biochemical assays for NODs. We have successfully expressed the nucleotide-binding domain of NOD2 using recombinant baculoviruses and purified the His6-tagged protein. A fluorescence polarization assay (FPA) will be established using FITC-conjugated ATP, analogous to the assay our laboratory previously reported for the NLR-family member, Nalp1 (NRLP1) (Faustin, B. et al., Molecular Cell, 25: 713-724, 2007). A similar assay will be devised for NOD1. This assay will identify any compounds that inhibit NOD1 or NOD2 by competing for ATP binding to the NACHT domain.

9. The NOD1 versus NOD2 cell-based and biochemical assays will be performed to determine whether compounds inhibit selective by either NOD1 or NOD2, versus having broad-spectrum cross-reactivity against these and possibly other members of the NLR family.

At the end of these secondary assays, we will have identified chemical inhibitors that operate selectively on the NOD1/NOD2-pathway for NF-κB activation, and we will have segregated them into 2 categories of compounds those that attack the ATP-binding site versus those that operate through other mechanisms. As usual, activity searches of the chemistry literature and databases (Scifinder, PubChem, IDdb3 abd the CBIS Vendor Compound Database) will also indicate whether any of our hits have previously been identified as inhibitors of NF-κB pathways or whether they have additional “off-target” activities about which we should be aware, because launching into detailed SAR and chemistry optimization.

### Example 2

### Assays and Compounds Related to NOD1 and NOD2 Modulation

### Example 2A

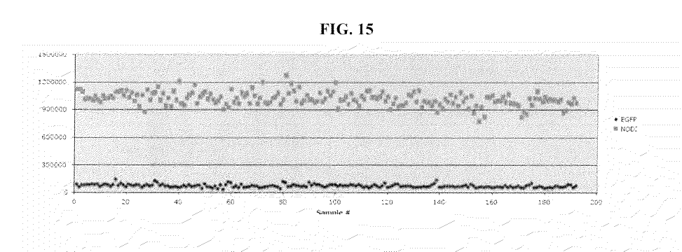

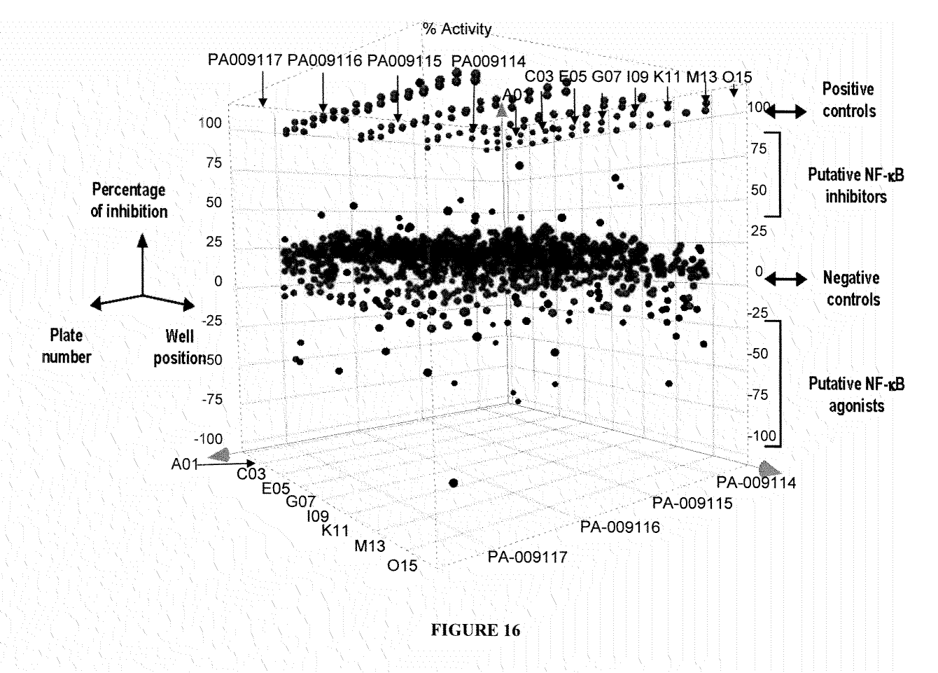

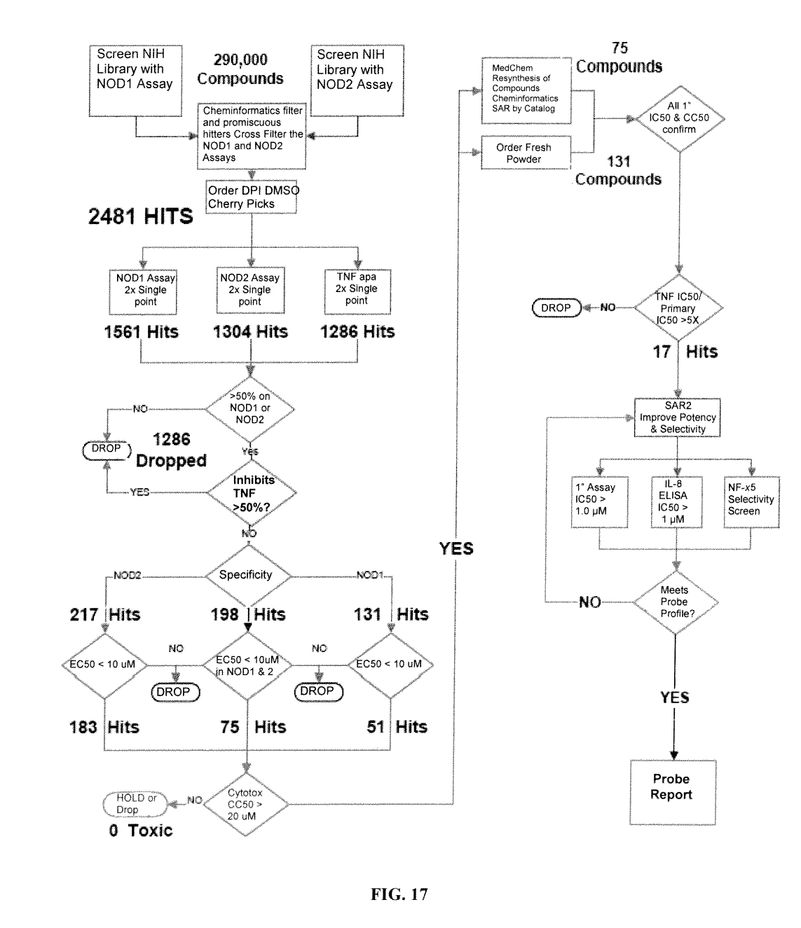

FIG. 17 illustrates the general triage used to prosecute actives in NOD1 and NOD2 primary assays, which then “tri”-furcate into NOD1 selective, NOD2 selective and NOD1/2 dual selective inhibitors. The right hand branch in FIG. 17 at the “Specificity” branchpoint” represent NOD1 selective inhibitors to follow up.

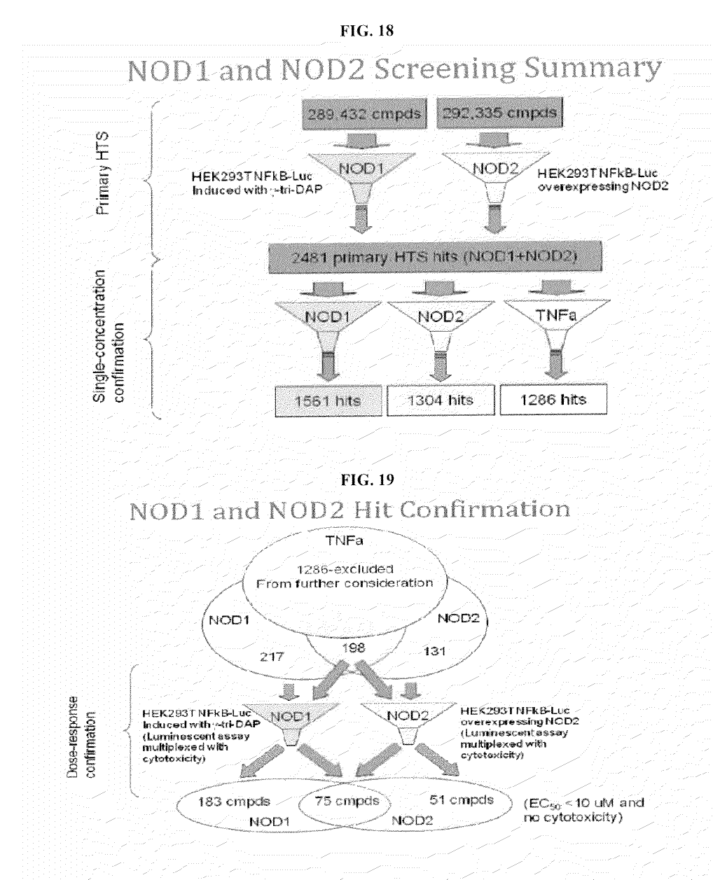

A library of approximately 290,000 compounds was tested in 2 assays: NOD1 and a NOD2-selective reporter assay. After further in silico screening by cheminformatics to eliminate historically promiscuous bioactives, 2481 hits with activity >50% at a single concentration point of 4 μM in either NOD1 or NOD2 were identified. Of these primary screening hits, 1561 were NOD1 hits 1304 were NOD2 hits (see FIG. 18).

DPI compounds were subsequently ordered for reconfirmation in single dose and dose response. The compounds were first confirmed in 4 μM single-point duplicate in the NOD1, NOD2 and TNFα assays. TNFα was used as a third filter assay to identify hits specific to TNFα, mediated NF-κB activation, which is putatively not NOD-mediated.

Hit totals for reconfirmation in single point actives were 217, 131 and 198 for NOD1, NOD2 and NOD1/2 respectively. 1286 compounds were identified as hits in the TNFα assay (>50% activity at 4 μM) and they were excluded from further consideration (see FIG. 19).

Reconfirmed DPI NOD1 and NOD2 actives were further assayed in dose response. To be considered active, compounds would fall into one of 3 bins: For a NOD1 active. IC₅₀s would have to fall below 10 μM with at least 10-told selectivity over NOD2. For NOD2. IC₅₀s would have to fall below 10 μM with at least 10-fold selectivity over NOD1. For dual activity we were looking for equipotency in NOD1 and NOD2 below 10 μM. All would have to show a clean cytotoxicity profile in alamar blue assay (<20 μM).

The total number of hits was further reduced upon testing in dose response to 183, 51 and 75 for NOD1, NOD2 and NOD1/2 respectively. At this stage, the alamar blue cytotoxicity assay was multiplexed in dose response with the NOD assays (see FIG. 19).

Chemistry and cheminformatics resources were then employed in the selection of both novel and chemically tractable molecules to pursue for a NOD1, NOD2 and NOD1/2 selective probe. Structures of interest and analogs thereof were either purchased as dry powders or, where unavailable, synthesized. In total, 75 structures were synthesized and 131 ordered though outside vendors. These constituted the SAR driving chemistries from which the NOD1 probe candidate and thirteen analogs emerged.

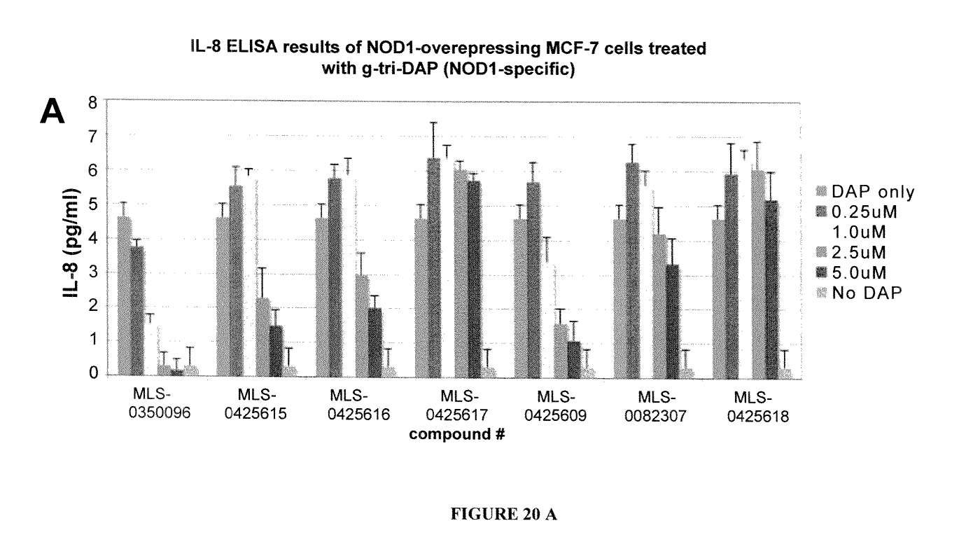

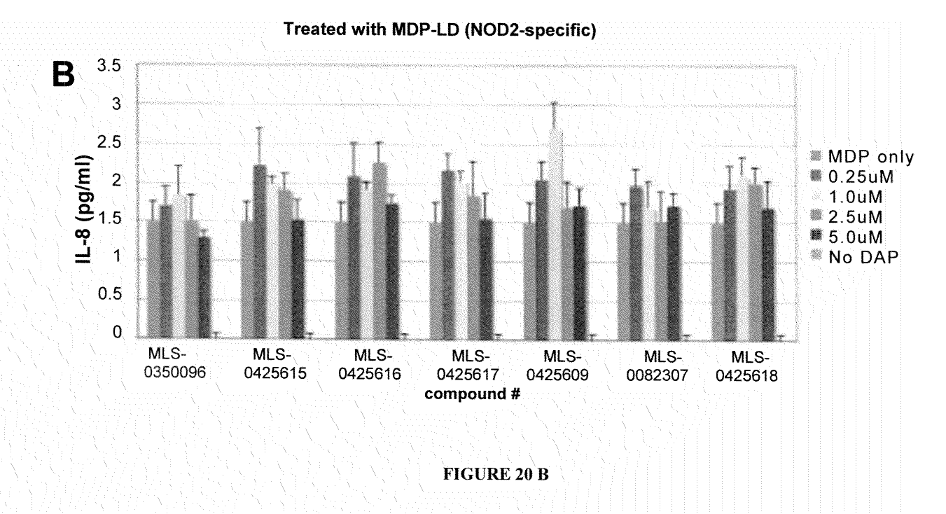

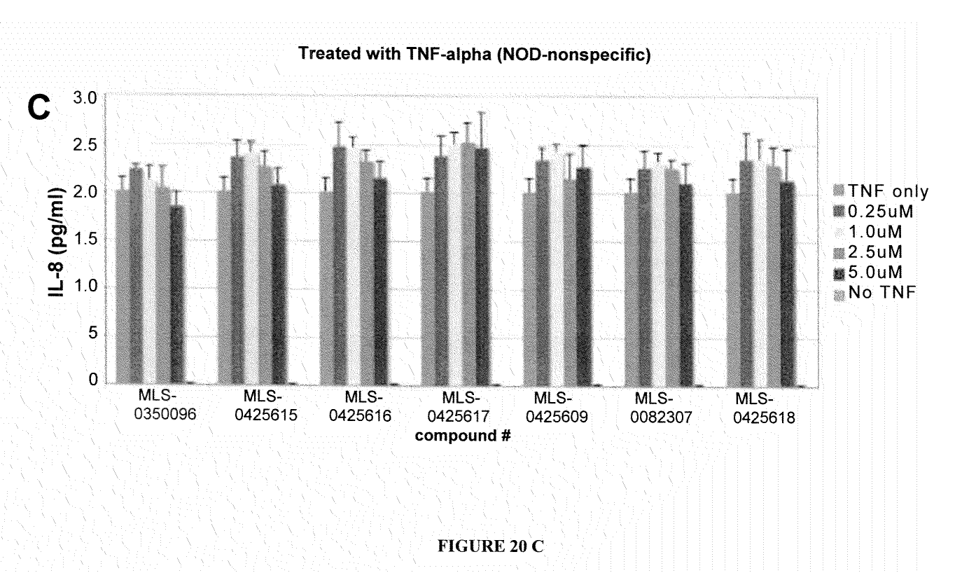

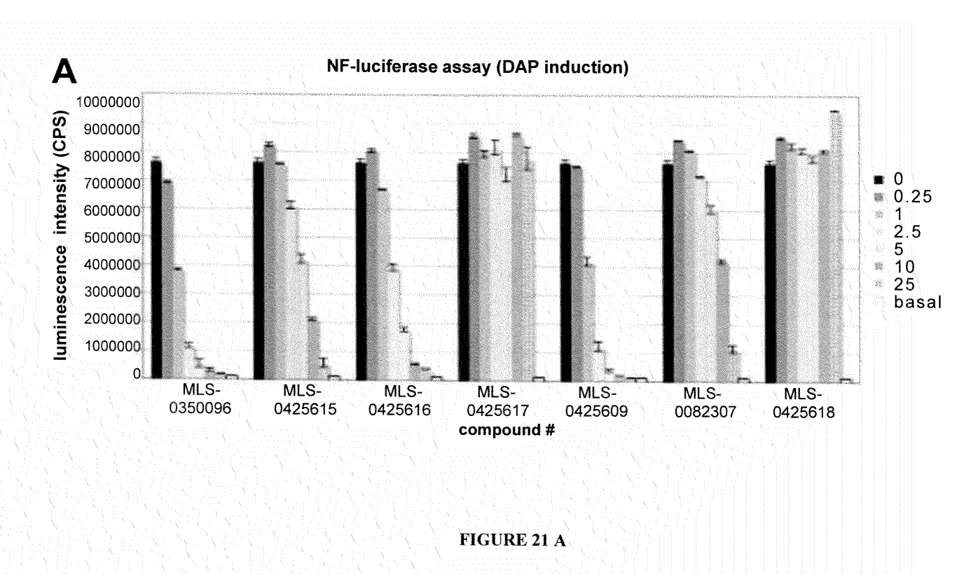

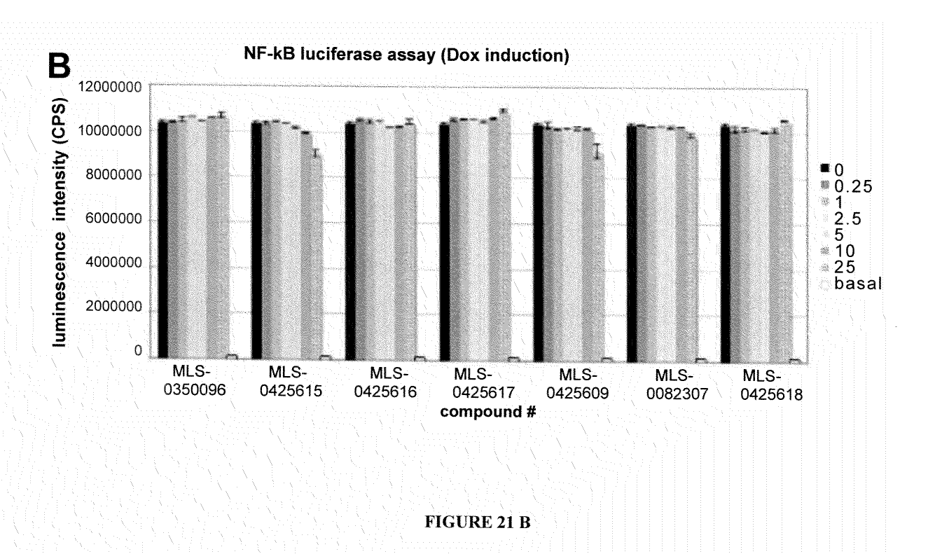

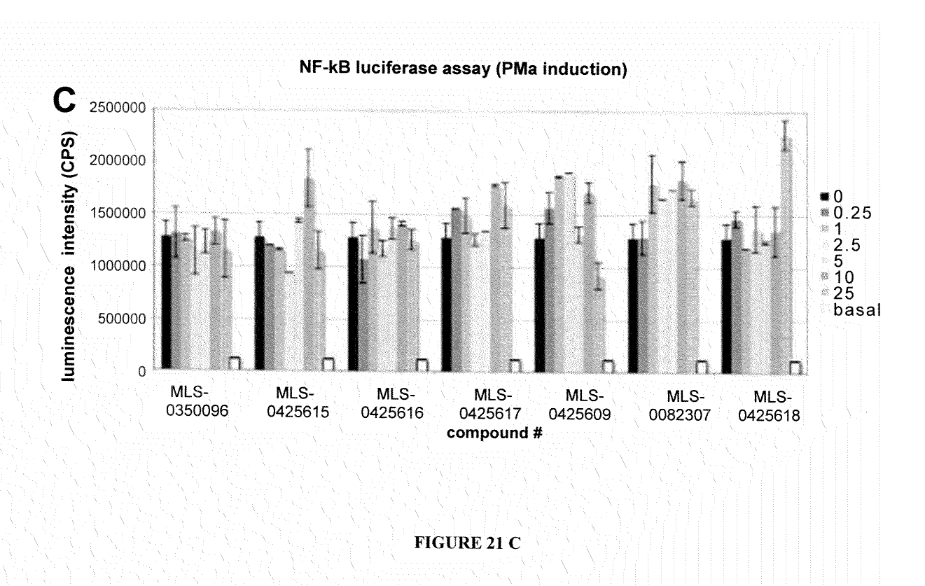

SAR testing of re-constituted powders encompassed dose response testing of compounds in four assays: NOD1, NOD2, TNFα, and alamar blue cytotoxicity (see FIG. 20). At this stage, the alamar blue cytotoxicity assay was multiplexed in dose response with the TNFα assay. Final probe selection, however, rested on the outcome of testing in a separate, biologically relevant functional assay, interleukin-8 (IL-8) secretion ELISA and on further selectivity testing in reporter assays using additional NF-κB pathway inducers (doxorubicin and PMA alongside the canonical NOD1 inducer gamma-tri-DAP) to eliminate these as possible targets of the testing agents. Tests were confirmed to be dose dependent inhibition of IL-8 secretion and inactivity of the probe in TNFα, PMA and doxorubicin induced NF-κB as well as inactivity in MDP induced (NOD2) mediated IL-8 release.

Compound 1 CID1088438 (MLS-0350096) (entry 1, Table 1) was identified through a high-throughput screening campaign involving 290,000 compounds as an active and NOD1-selective scaffold.

The probe molecule CID1088438 selectively (>40-fold) inhibits NOD1 dependent activation of NF-κB pathways as ascertained through γ-tri-DAP stimulated luciferase signaling in a NF-κB-linked reporter assay in HEK293T cells containing endogenous NOD1 levels with submicromolar potency (0.52 μM IC₅₀), while not inhibiting MDP stimulated (NOD2-dependent) signaling in both reporter cell lines containing both low and overexpressed NOD2 proteins. The probe molecule is selective over the non-NOD stimulated pathways (TNFα stimulation) of NF-κB in these reporter assays.

Furthermore, the probe molecule and closely related analogs, appear also to selectively inhibit the biologically relevant terminal effect of NOD1 (γ-tri-DAP) dependent NF-κB activation (1st panel below), namely IL-8 secretion, but not NOD2 dependent (2nd panel below), nor TNFα dependent (3rd panel below) IL-8 secretion in biologically relevant MCF-7 cells as determined by IL-8 ELISA kits of cell culture supernatants.

Finally, the probe molecule and close analogs also are selective for NOD1 dependent activation of NF-κB as they do not inhibit doxorubicin (DNA damage) and PMA/ionomycin (phorbol ester/ionophore) induced pathways.

### Example 2B

This example describes an example of the disclosed methods used to screen for NOD modulators. A primary screen assay measured the luciferase activity induced in the cell line 293T-kB-LV-LUC upon exposure to Ala-γ-Glu-diaminopimelic acid (γ-tri-DAP), which acts through the NOD1 signaling pathways to activate NT-κB, thus inducing an integrated NF-κB dependent luciferase expression cassette. The cell-based HTS assay utilized NF-κB-mediated luciferase reporter gene activity as a measure of NOD1 modulation. The assay used a luminescent readout.

### Assay Materials

1. HEK-293-T NF-κB-Luc cell line obtained from the assay provider's laboratory.
2. γ-tri-DAP (Ana Spec cat #60774) obtained from assay provider's laboratory.
3. SteadyGlo (Promega).

TABLE 2
Reagents used for the uHTS experiments

| Reagent | Vendor |
|---|---|
| Human Embryonic Kidney Cells stably transduced | Cell from AP, |
| with a 5X NF-κB RE (response elements) upstream | scaled up |
| of a firefly luciferase cassette | |
| Ala-γ-Glu-diaminopimelic acid - inducer of NOD1 | Donated by AP |
| pathway to activation of NF-κB | |
| Commercial lumigenic Luciferase substrate | Perkin-Elmer |
| (Britelite ™) | |

TABLE 3
Assay Name and Type.

| | | | | Assay | Assay Detection |
|---|---|---|---|---|---|
| PubChemBioAssay Name | AIDs | Probe Type | Assay Type | Format | & well format |
| Summary assay for the | 1575 | Inhibitor | Summary | N/A | N/A |
| identification or compounds | | | | | |
| that inhibit NOD1 [Summary] | | | | | |
| uHTS luminescence assay for | 1578 | Inhibitor | Primary | Cell-based | luminescence & |
| the identification of | | | | | 1536 |
| compounds that inhibit NOD1 | | | | | |
| [Confirmatory] | | | | | |
| uHTS luminescence assay for | 1566 | Inhibitor | Counterscreen for | Cell-based | luminescence & |
| the identification of | | | NOD1 (also | | 1536 |
| compounds that inhibit NOD2 | | | NOD2 Primary) | | |
| [Confirmatory] | | | | | |
| HTS assay for identification of | 1852 | Inhibitor | Counterscreen | Cell-based | luminescence & |
| inhibitors of TNFα-specific | | | | | 1536 |
| NF-κB induction | | | | | |
| uHTS Fluorescence assay for | 1849 | Inhibitor | Cytotoxicity | Cell-based | luminescence & |
| the identification of cytotoxic | | | Counterscreen | | 1536 |
| compounds among | | | | | |
| compounds active in NOD1 | | | | | |
| cell inhibition assay | | | | | |
| [Confirmatory] | | | | | |
| uHTS luminescence assay for | 2001 | Inhibitor | Counterscreen | Cell-based | luminescence & |
| the identification of | | | | | 1536 |
| compounds that inhibit NOD2 | | | | | |
| in MDP treated cells. | | | | | |
| [Confirmatory] | | | | | |
| SAR analysis of compounds | 2333 | Inhibitor | SAR | Cell-based | Luminescence & |
| that inhibit NOD1 | | | | | 384 |
| [Confirmatory] | | | | | |
| SAR analysis of compounds | 2334 | Inhibitor | SAR | Cell-based | Luminescence & |
| that inhibit NOD2 | | | | | 384 |
| [Confirmatory] | | | | | |
| SAR analysis of inhibitors of | 2337 | Inhibitor | SAR | Cell-based | Luminescence & |
| TNFα specific NF-κB | | | Counterscreen | | 1536 |
| induction [Confirmatory] | | | | | |
| SAR analysis of compounds | 2335 | Inhibitor | SAR Cytotoxicity | Cell-based | luminescence & |
| that are cytotoxic to HEK293 | | | Counterscreen | | 1536 |
| [Confirmatory] | | | | | |
| SAR analysis of muramyl | 2260 | Inhibitor | Secondary Assay | Cell-based | Absorbance (at |
| dipeptide (MDP) induced IL-8 | | | for specificity | | 450 nm) & 96 |
| secretion in MCF-7/NOD2 | | | | | |
| cells. | | | | | |
| [Confirmatory] | | | | | |
| SAR analysis of tumor | 2245 | Inhibitor | Secondary Assay | Cell-based | Absorbance |
| necrosis factor alpha (TNFα) | | | for specificity | | (ELISA) of cell |
| induced IL-8 secretion in | | | | | extracts & 96 |
| MCF-7/NOD1 cells | | | | | |
| [Confirmatory] | | | | | |
| SAR analysis of GM-Tri-DAP | 2250 | Inhibitor | Secondary Assay | Cell-based | Absorbance |
| induced IL-8 secretion in | | | for specificity | | (ELISA) of cell |
| MCF-7/NOD1 cells | | | | | extracts & 96 |
| [Confirmatory] | | | | | |
| SAR analysis of NF-κB | 2264 | Inhibitor | Secondary Assay | Cell-based | luminescence & |
| dependent luciferase using | | | for specificity | | 96 |
| DAP as an inducer | | | | | |
| [Confirmatory] | | | | | |
| SAR analysis of NF-κB | 2261 | Inhibitor | Secondary Assay | Cell-based | luminescence & |
| dependent luciferase using | | | for specificity | | 96 |
| PMA/Ionomycin as an inducer | | | | | |
| [Confirmatory] | | | | | |
| SAR analysis of NF-κB | 2255 | Inhibitor | Secondary Assay | Cell-based | luminescence & |
| dependent luciferase using | | | for specificity | | 96 |
| Doxorucibin as an inducer | | | | | |
| [Confirmatory] | | | | | |

The following uHTS protocol was implemented at single point concentration confirmation:

1. Day 1
1. Harvest HEK-293-T NF-κB-Luc at 100% confluency
2. Dispense 3 μL (6000 cells)/well to every well of a 1536 TC-treated white plate (Corning #3727).
3. Spin down plates at 1000 rpm for 1 min in an Eppendorf 5810 centrifuge.
4. Using a HighRes biosolution pintool equipped with V&P Scientific pins, stamp 10 nl of 2 mM compounds in DMSO (col 5-48) and 10 nl DMSO controls (col 1-4) to plates
5. Lid Plates. Incubate cells for 1 hour at room temp.
6. Dispense 2 μL/well of γ-tri-DAP (1.875 μg/mL) in assay media containing 1.375% DMSO to columns 3-48.
7. Spin down plates 30 sec in an Eppendorf 5810 centrifuge.
8. Lid Plates. Incubate overnight (16 hours) in 37° C. 5% CO₂ incubator.

2. Day 2
1. Equibrate plates to room temperature for 10 mins.
2. Add 3 μL SteadyGlo well with Multidrop
3. Spin plates for 10 secs in a Velocity11 VSpin, shake for 30 secs.
4. Incubate plates for 20 mins at room temperature.
5. Read luminescence on Perkin. Elmer Viewlux™.
   The average Z′ for the screen was 0.6, the signal to background was 11.1, signal to noise was 78.6 and signal to window was 6.0.

### Rationale for Confirmatory, Counter and Selectivity Assays

Past experience with cell-based assays for NF-κB and pilot LOPAC screen of NOD1 and with NOD2, a substantial number of initial hits were projected for NOD1 (˜6800 hits for NOD1 and ˜1400 hits for NOD2 inhibitors. Therefore, PubChem comparisons for existing NF-κB firefly luciferase data, as well as promiscuous and generally toxic compounds filters were used before any retests of compounds.

### Confirmation Assays

The initial confirmatory screens were obtained from full dose-response of compounds from solvated DPI compounds to confirm activity seen first in test agents from screening library. The criteria were to have NOD1 active IC₅₀s below 10 μM with at least 10-fold selectivity over NOD2. For NOD2, IC₅₀s would have to fall below 10 μM with at least 10-fold selectivity over NOD1. For dual activity we were looking for equipotency in NOD1 and NOD2 below 10 μM. Compounds that did met these criteria and showed well-behaved plots with Hill slopes between 0.7 and 1.4 were progressed to next stage. NOD1 second level confirmatory screens were obtained from full dose-response of compounds from dry powders in NOD1 and NOD2. Compounds fulfilling the above mentioned criteria were advanced to secondary assays.

### Counterscreen Assays

Counterscreens consisted of an alamar blue cytotoxicity filter and a dose response assay to identify hits specific to tumor necrosis factor alpha (TNFα), -modulated NF-κB. A positive in a cytotoxicity assay invalidates as false positive a positive from the same compound in the NOD and/or TNFα, assays. Since multiple cellular stimuli acting through various pathways lead to NF-κB induction, the TNFα assay is designed to identify hits specific to TNFα modulated pathways (non-NOD modulated).

### Secondary Assays

Secondary assays performed to establish that (1) the compounds do actually inhibit the biologically relevant downstream effectors of NOD1 stimulated pathway (IL-8 secretion) and are not just the reporter pathway, and (2) selectively inhibit the NOD1 dependent pathway to NF-κB activation in other cell lines. The AIDs for these assays are summarized in Table 4 below:

TABLE 4
Summary of the secondary assays used in NOD1 studies.

| Assay Name | AID | Assay Type |
|---|---|---|
| NOD1: IL-8 secretion | 2250 | Secondary |
| NOD2: IL-8 | 2260 | Secondary |
| secretion | | |
| TNF-α: IL-8 secretion | 2245 | Secondary |
| DAP: NF-κB selectivity | 2264 | Secondary |
| PMA: NF-κB selectivity | 2261 | Secondary |
| DOX: NF-κB selectivity | 2255 | Secondary |

NOD1: IL-8 secretion (AID 2250): γ-tri-DAP induction of human breast cancer epithelial cell lines MCF-7 expressing NOD1, combined with small doses of cycloheximide (CHX), specifically induces IL-8 production and release (da Silva Correia et al. 2007; da Silva Correia et al. 2006). NOD1 specifically detects Gamma-Tri-DAP, a tripeptide motif found in Gram-negative bacterial peptidoglycan, resulting in activation of the transcription factor NF-κB pathway (Girardin et al. 2003).

NOD2: IL-8 secretion (AID 2260): muramyl dipeptide (MDP) induction of human breast cancer epithelial cell lines MCF-7 over-expressing NOD2 combined with small doses of cycloheximide (CHX), specifically induces IL-8 production and release (da Silva Correia et al. 2007; da Silva Correia et al. 2006). NOD2 is a general sensor of peptidoglycan through the recognition of muramyl dipeptide (MDP), the minimal bioactive peptidoglycan motif common to all bacteria (Girardin et al. 2003).

TNFα: IL-8 secretion (AID2245): The assay uses tumor necrosis factor alpha (TNFα), a canonical NF-κB inducer, and is designed for identification of hits specific to TNFα-modulated pathways in MCF-7/NOD1 cells (Girardin et al. 2003). NOD1 specific inhibitors are not expected to affect this pathway (i.e. IL-8 secretion). In all cases secreted IL-8 was quantified with 96-well ELISA kit for IL-8 (BD Biosciences) using a SpectraMax 190 to measure absorbance at 570 nm.

DAP: NF-κB selectivity (AID2264); NF-κB selectivity (AID2261); DOX: NF-κB selectivity (AID2255): All three of these assays are cell-based confirmatory assay that utilizes NF-κB-mediated luciferase reporter gene activity in an engineered cell line (HEK-293-T NF-κB-Luc), as a measure of NF-κB activation via NOD1 (DAP) modulation, general activation (PMA/ionomycin), and DNA damage (Dox) pathways. The assays use a luminescent readout my measuring luciferase activity with Steady Glow luciferase reagents.

A library of approximately 290,000 compounds was tested in 2 assays: NOD1 and a NOD2-selective reporter assay. After further in silico screening by cheminformatics to eliminate historically promiscuous bioactives, 2481 hits with activity >50% at a single concentration point of 4 μM in either NOD1 or NOD2 were identified. Of these primary screening hits, 1536 were NOD1 hits 1304 were NOD2 hits.

DPI compounds were subsequently ordered for reconfirmation in single dose and dose response. The compounds were first confirmed in 4 μM single-point duplicate in the NOD1, NOD2 and TNFα assays. TNFα was used as a third filter assay to identify hits specific to TNFα mediated NF-κB activation, which is putatively not NOD-mediated.

Hit totals for reconfirmation in single point actives were 217, 131 and 198 for NOD1, NOD2 and NOD1/2 respectively. 1236 compounds were identified as hits in the TNFα0 assay (>50% activity at 4 μM) and they were excluded from further consideration.

Reconfirmed DPI NOD1 and NOD2 actives were further assayed in dose response. To be considered active, compounds would fall into one of 3 bins: For a NOD1 active, IC₅₀s would have to fall below 10 μM with at least 10-fold selectivity over NOD2. For NOD2, IC₅₀s would have to fall below 10 μM with at least 10-fold selectivity over NOD1. For dual activity we were looking for equipotency in NOD1 and NOD2 below 10 μM. All would have to show a clean cytotoxicity profile in alamar blue assay (<20 μM).

The total number of hits was further reduced upon testing in dose response to 183, 51 and 75 for NOD1, NOD2 and NOD1/2 respectively. At this stave, the alamar blue cytotoxicity assay was multiplexed in dose response with the NOD assays.

Chemistry and cheminformatics resources were then employed in the selection of both novel and chemically tractable molecules to pursue for a NOD1, NOD2 and NOD1/2 selective probe. Structures of interest and analogs thereof were either purchased as dry powders or, where unavailable, synthesized by BIMR. In total, 75 structures were synthesized and 131 ordered though outside vendors. These constituted the SAR driving chemistries from which the NOD1 probe candidate and thirteen analogs emerged.

SAR testing of re-constituted powders encompassed dose response testing of compounds in tour assays: NOD1, NOD2, TNFα, and alamar blue cytotoxicity. At this stage, the alamar blue cytotoxicity assay was multiplexed in dose response with the TNFα assay. Final probe selection, however, rested on the outcome of testing in a separate, biologically relevant functional assay, interleukin-8 (IL-8) secretion ELISA and on further selectivity testing in reporter assays using additional NF-κB pathway inducers (doxorubicin and PMA alongside the canonical NOD1 inducer gamma-tri-DAP) to eliminate these as possible targets of our testing agents. Dose dependent inhibition of IL-8 secretion and inactivity of the probe in TNFα, PMA and doxorubicin induced NF-κB as well as inactivity in MDP induced (NOD2) mediated II-8 release has been repeatedly confirmed.

### Example 3

### Synthesis and Properties of Utilized Compounds

### Synthesizing Compound 1

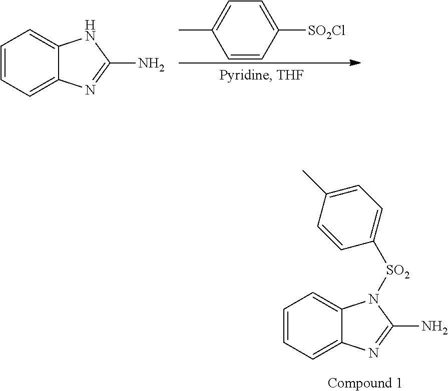

A round-bottom flask was charged with 2-aminobenzimidazole (100.0 mg, 0.75 mmol) and pyridine (0.31 mL) at room temperature. p-Toluenesulfonyl chloride (147.5 mg, 0.77 mmol, 1.03 equiv.) was added in one portion and the resulting cloudy solution was stirred overnight to form a thick mass. Tetrahydrofuran (THF) (0.5 mL) was added to aide solubility and the crude mixture was loaded on a preparatory TLC plate using straight ethyl acetate as eluent. The product was removed from silica gel by using 10% MeOH-Ethylacetate and was isolated as a tan solid (75.5 mg, 35% yield): ¹H NMR (400 MHz, DMSO-d₆) δ 8.02-7.86 (m, 2H), 7.65 (dt, J=8.0, 0.9 Hz, 1H), 7.50-7.39 (m, 2H), 7.19-7.07 (m, 4H), 7.07-6.96 (m, 1H), 2.34 (d, J=7.1 Hz, 3H). ¹³C (100 MHz, DMSO-d₆) δ 152.2, 146.4, 142.8, 133.8, 130.5, 130.1, 126.8, 124.7, 120.6, 116.0, 112.2, 21.2. Melting point: 191-192° C. (with decomposition). Purity: >95% (HPLC). Mass Spec: ESI m/z 288 [M+H].

### Physicochemical Properties of Compound 1

Compound 1 exhibited low solubility and high permeability at the three pH levels tested. It exhibits high plasma protein binding (both human and mouse). It has high stability in both human and mouse plasma. It shows low stability in the presence of mouse microsomes but moderate stability in human microsomes. The probe compound has a LD₅₀>50 μM towards Fa2N-4 immortalized human hepatocytes.

TABLE 5

| | | | Plasma Protein Binding | | Plasma | Microsomal | |
|---|---|---|---|---|---|---|---|
| | Solubility | Permeability | (% Bound) | | Stability | Stability | |
| | (μg/mL)^{a} | (×10⁻⁶ cm/s) | Human | Mouse | (% Remaining | (% Remaining | Toxicity^{b} |
| Compound | pH | pH | 1 μM/ | 1 μM/ | at 3 hrs) | at 1 hr) | (LD50, |
| ID | 5.0/6.2/7.4 | 5.0/6.2/7.4 | 10 μM | 10 μM | Human/Mouse | Human/Mouse | μM) |
| MLS- | 2.0/1.7/2.0 | 491/562/382 | 97.74/97.45 | 95.45/94.91 | 100/100 | 41.78/0.83 | >50 |
| 0350096 | | | | | | | |

^{a}in aqueous buffer
^{b}towards Fa2N-4 immortalized human hepatocytes

TABLE 6
Comparative data showing probe specificity for target in biologically relevant assays
(this table summarized the data from figures in text above from the secondary assay)

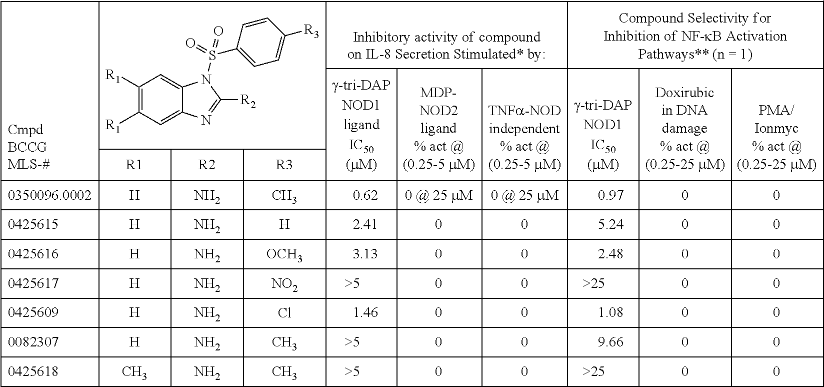

*For the IL-8 Secretion assays all compounds were tested at 0.25, 1.0, 2.5 & 5.0 μM; unless otherwise noted the highest concentration tested is >x where x is the highest tested concentration
**For the NF-κB Pathway specificity assays all compounds were tested at 0.25, 1.0, 2.5, 5.0, 10 & 25 μM
Where IC₅₀ values are listed, they are fitted by a 4-parameter fit using PRISM software

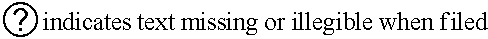

### Example 4

### In Vivo RACE Assay

In vivo RACE assay data was obtained for compound 1:

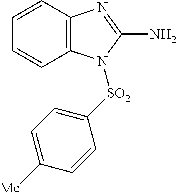

in conjunction with quantitative bioanalytical analysis to test its pharmacokinetics in wild type mice. Mice dosed with the compound IP (30 mg/kg and 15 mg/kg) exhibited significant compound exposure at T=20 min which rapidly decreased by T=120 min. Mice dosed IP with the compound (30 mg/kg) exhibited a higher compound exposure than mice dosed IP (15 mg/kg).

| Dose | Concentration (uM) | Concentration (uM) |
|---|---|---|
| (mg/kg) | 20 min | 120 min |
| 15 | 1.58 ± 0.06 | 0.12 ± 0.05 |
| 30 | 5.24 ± 0.62 | 0.54 ± 0.04 |

The samples showed the presence of a potential oxidation metabolite [M+16]. The compound was also found to be well tolerated at this dose with no signs of toxicity.

### Example 5

### XIAP Mediates NOD Signaling Via Interaction with RIP2

NOD1 and NOD2, share structural and functional characteristics. Both, NOD1 and NOD2, contain C-terminal leucine-rich repeats (LRRs) thought to act as receptors for pathogen-derived molecules, a central nucleotide-binding oligomerization domain (NACHT) (Bell et al. (2003), Opitz et al. (2004)), and N-terminal caspase recruitment domains (CARDs) that associate with down-stream signaling proteins (Inohara et al. (1999), Ogura et al. (2001)). NODs activation is stimulated by bacterial peptides derived from peptidoglycans, with diaminopimelic acid (DAP) stimulating NOD1 (Chamaillard et al. (2003), Girardin et al. (2003a)), and muramyl dipeptide (MDP) activating NOD2 (Girardin et al. (2003b), Inohara et al. (2003)). Upon recognition of these ligands, oligomerization of the NACHT domains initiates the recruitment of interacting proteins, binding the serine/threonine protein kinase RIP2/CARDIAK/RICK via CARD-CARD-interactions (Inohara et al. (1999), Ogura et al. (2001)). RIP2 is critical for NF-κB activation induced by NOD1 and NOD2 (Kobayashi et al. (2002)). RIP2 not only binds to NOD1 and NOD2 via CARD-CARD interactions, but it also associates with other signaling proteins independently of the CARD, including members of the TNFR-associated factor (TRAF) family and members of the inhibitor of apoptosis protein (IAP) family, cIAP-1 and cIAP-2 (McCarthy et al. (1998), Thome et al. (1998)). IAP-family proteins play prominent roles in regulating programmed cell death by virtue of their ability to bind caspases (Eckelman et al. (2006), Deveraux et al. (1998), Deveraux et al. (1997), Roy et al. (1997)), intracellular cysteine proteases responsible for apoptosis. A common structural feature of the IAPs is the presence of one or more baculoviral IAP-repeat (BIR) domains, which serve as scaffolds for protein interactions (Sun et al. (1999)). One of the most extensively investigated members of the IAP-family is X-linked IAP (XIAP). XIAP contains three BIR domains (Duckett et al. (1996)), followed by a ubiquitin binding domain (UBA) (Gyrd-Hansen et al. (2008)) and a C-terminal RING that functions as E3-Ligase promoting ubiquitination and subsequent proteasomal degradation of distinct target proteins (Yang et al. (2000)). In additional to its anti-apoptotic role as a caspase inhibitor, XIAP functions in certain signal transduction processes, which include activation of MAPKs (Sanna et al. (1998)) and NF-κB through interactions of TAB1/TAK1 with its BIR1 domain (Lu et al. (2007)). Studies demonstrate that flies depleted by shRNA of Drosophila IAP-2 (DIAP2) fail to activate NF-κB in response to bacterial challenge with Escherichia coli and show decreased survival rates when exposed to Enterobacter cloacae (Gesellchen et al. (2005). Huh et al. (2007)). There is evidence that XIAP might be involved in NF-κB and JNK activation induced by TLRs and NLRs during infection with Listeria monocytogenes (Bauler et al. (2008)). There results here show that XIAP is required at least in certain types of epithelial cells for NF-κB activation induced by NOD1 and NOD2, and demonstrate that XIAP binds RIP2 thereby associating with NOD1/NOD2 signaling complexes.

In this example, it is shown that NOD signaling is dependent on XIAP, a member of the inhibitor of apoptosis protein (IAP) family. Cells deficient in XIAP exhibit a marked reduction in NF-κB activation induced by microbial NOD ligands and by over-expression of NOD1 or NOD2. Moreover, this example shows that XIAP interacts with RIP2 via its BIR2 domain, which could be disrupted by XIAP antagonists SMAC and SMAC-mimicking compounds. Both NOD1 and NOD2 associated with XIAP in a RIP2-dependent manner, indicating that XIAP associates with the NOD signalosome. Taken together, these results indicate that XIAP is involved in regulating innate immune responses by interacting with NOD1 and NOD2 through interaction with RIP2.

### Results

XIAP Is Required for NOD Signaling. Epithelial cells of the intestinal track are a first line of defense against many microorganisms. Human tumor cell lines derived from colonic epithelium in which the XIAP gene had been ablated by homologous recombination were used to ask whether XIAP is required for cellular responses to synthetic NOD1 or NOD2 ligands. Accordingly, isogenic pairs of XIAP−/− and XIAP−/− HCT116 and DLD-1 cells were stimulated for 24 h with NOD1 and NOD2 ligands, L-Ala-γ-D-Glu-mDAP (DAP) and muramyl dipeptide (MDP), respectively, then Interleukin-8 (IL-8) production was measured (FIGS. 22A and B). Both DAP and MDP induced increases in IL-8 production in the wild-type HCT116 and DLD-1 cells, with MDP more potent than DAP. In contrast, neither of these NOD ligands induced IL-8 production in cultures of XIAP-deficient HCT116 and DLD1. Whereas XIAP−/− cells failed to respond to NOD ligands, they remained responsive to TNF, which induced robust IL-8 production.

The observation that XIAP gene knock-out impairs NOD-signaling was further confirmed by quantitative RT-PCR analysis of the NF-κB target genes IκBα and IL-8, detecting decreased levels of IκBα and IL-8 mRNAs in XIAP-deficient HCT116 cells compared with wild-type HCT116 cells following stimulation with MDP or DAP (FIG. 22C). In contrast, TNF-α induced expression of these NF-κB target genes comparably in XIAP−/− and XIAP−/− cells. Similar observations were made using a NF-κB reporter gene to monitor responses to NOD ligands. In XIAP−/− HCT116 cells, stimulation with MDP induced increases in NF-κB reporter gene activity (FIG. 22D). In contrast, MDP and DAP failed to stimulate NF-κB reporter gene activity in XIAP−/− HCT116 cells. Transfecting XIAP−/− HCT116 cells with a plasmid encoding XIAP (FIG. 22D) restored responsiveness to NOD ligands. Stimulating the same cells with suboptimal concentrations of TNF-α served as a control, showing XIAP-independent activation of NF-κB. To explore the role of XIAP by an alternative approach, shRNA vectors were used to knock-down XIAP expression levels rather than gene ablation by homologous recombination. NF-κB activity was measured in control and XIAP knock-down (KD) HEK293T-cells stably expressing a NF-κB-driven luciferase reporter gene. Consistent with the observations in HCT116 and DLD-1 cells, MDP and DAP failed to activate NF-κB in cells deficient for XIAP when compared with the control vector-treated cells. In contrast, NF-κB activity was similarly induced in control vector- and XIAP shRNA-treated 293T cells after stimulation with other NF-κB inducers such as doxorubicin, PMA/ionomycin and TNF-α (FIG. 72E). In fact, PMA/ionomycin stimulated NF-κB reporter gene activity better in MAP KD cells.

NOD1 and NOD2 Induced NF-κB Activation Depends on XIAP. To further explore the role of XIAP in NOD signaling, NF-κB activity was induced by gene transfer mediated over-expression of NOD1, or NOD2, rather than using synthetic ligands to activate the endogenous proteins. HCT116 XIAP−/− or XIAP−/− cells were transfected with increasing amounts of either myc-NOD1 or -NOD2 plasmids along with a NF-κB-driven luciferase reporter gene plasmid. Both NOD1 and NOD2 induced increases in NF-κB reporter gene activity in a dose-dependent manner, whereas no increase in NF-κB activity was observed in XIAP-deficient cells (FIGS. 23A and B). Reconstitution experiments in which XIAP−/− HCT116 cells were transfected with a plasmid encoding FLAG-XIAP showed restoration of NOD1 and NOD2-induced NF-κB activity (FIG. 23C).

To confirm these observations by an alternative method in another cell line, HEK293T cells stably over-expressing NOD1 or NOD2 and containing a NF-κB-responsive luciferase reporter gene were infected acutely with XIAP shRNA lentivirus to achieve reductions in XIAP protein. NF-κB reporter gene activity driven by stable NOD1 or NOD2 over-expression was significantly reduced in these cells treated with XIAP shRNA virus compared with control virus (FIG. 23D), thus corroborating the results obtained with XIAP knock-out cell lines. Similar results were obtained in experiments where NOD1 and NOD2 were over-expressed by transient transfection (FIGS. 23E and F) or where XIAP was stably knocked down using shRNA (FIGS. 23G and H).

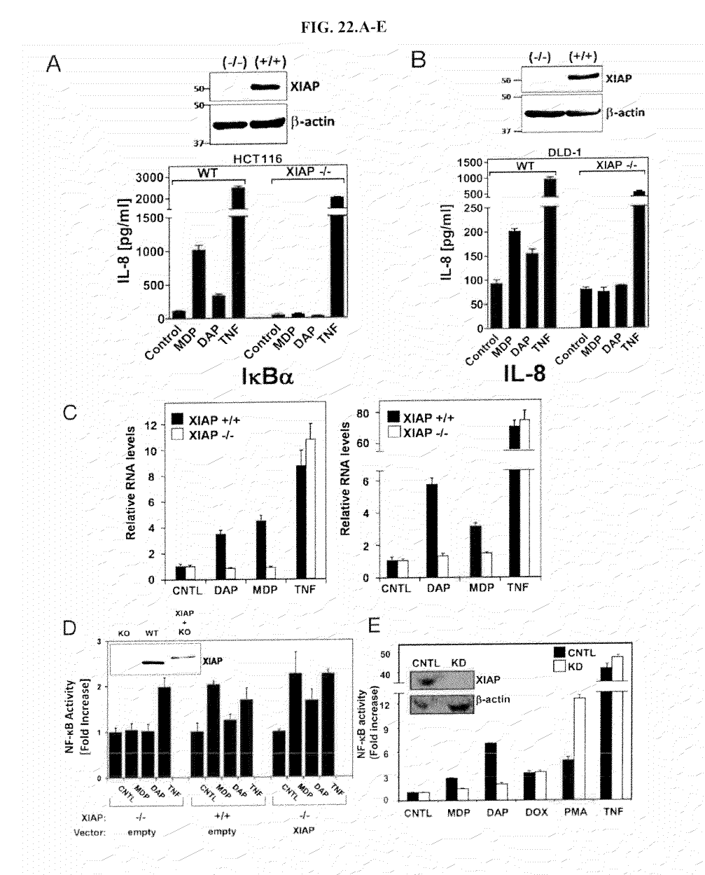

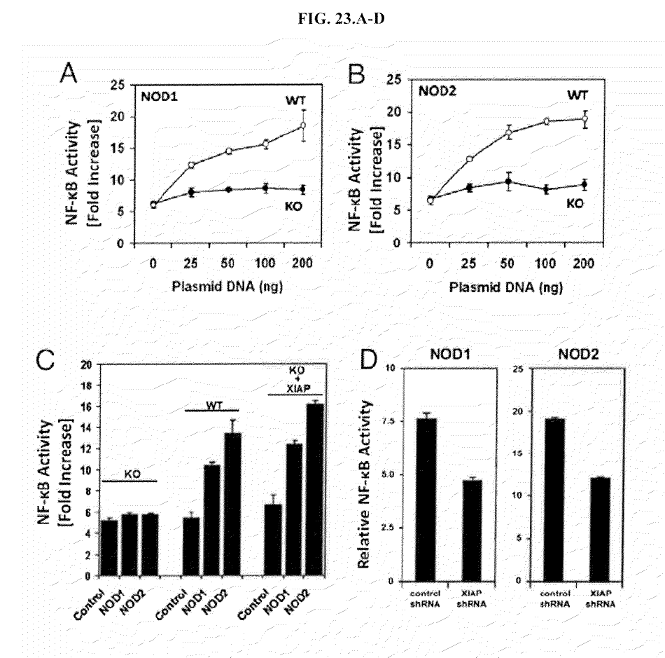

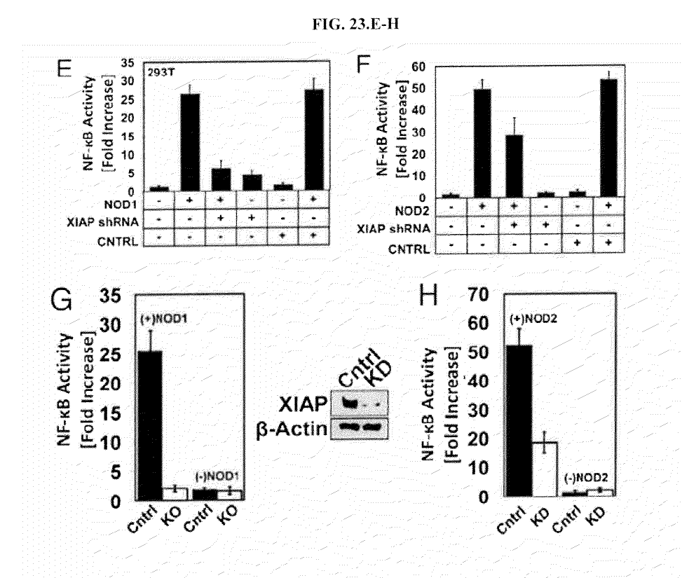

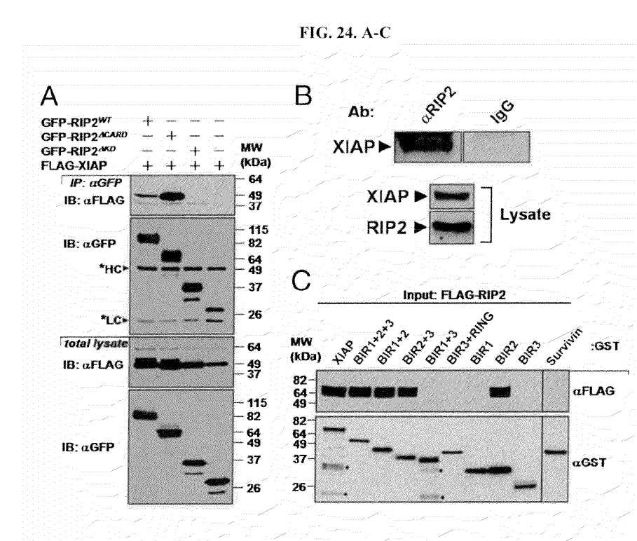

XIAP Directly Interacts with RIP2. The protein kinase and adapter protein RIP2 is a known contributor to NOD signaling, which interacts with NOD1 and NOD2 via CARD-CARD interactions (Kobayashi et al. (2002)). RIP2 has also been reported to associate with c-IAP1 and c-IAP2 (Bertrand (2009)). To investigate if RIP2 similarly interacts with XIAP, co-immunoprecipitation (co-IP) assays were performed using HEK293T cells expressing FLAG-XIAP and GFP-RIP2 by transfection. In addition, interactions of XIAP with full-length RIP2 and mutant versions of RIP2 lacking either the N-terminal CARD domain or the C-terminal kinase domain (KD) of RIP2 were compared. XIAP demonstrated binding to both full-length RIP2 and the RIP2ΔCARD but not to RIP2ΔKD (FIG. 24A). The interaction of endogenous XIAP with endogenous RIP2 was also demonstrated by co-IP using lysates of THP-1 monocytes, and anti-RIP2 antibodies for immunoprecipitation, showing that XIAP protein is recovered in immunoprecipitates generated using anti-RIP2 but not control antibody (FIG. 24B).

To further elucidate which domain of XIAP mediates binding to RIP2, in vitro protein binding studies were performed by the GST pull-down method, using a panel of GST-fusion proteins containing a variety of fragments of XIAP and incubating with lysates from HEK293T cells transfected with FLAG-RIP2. FLAG-RIP2 bound to fragments of XIAP containing the BIR2 domain, including a fragment comprised only of the BIR2 domain, whereas all fragments lacking the BIR2 domain failed to bind (FIG. 24C). In contrast, none of GST fusion proteins displayed interactions with a control protein, FLAG-SIP. Thus, the BIR2 domain of XIAP is both necessary and sufficient for RIP2 binding.

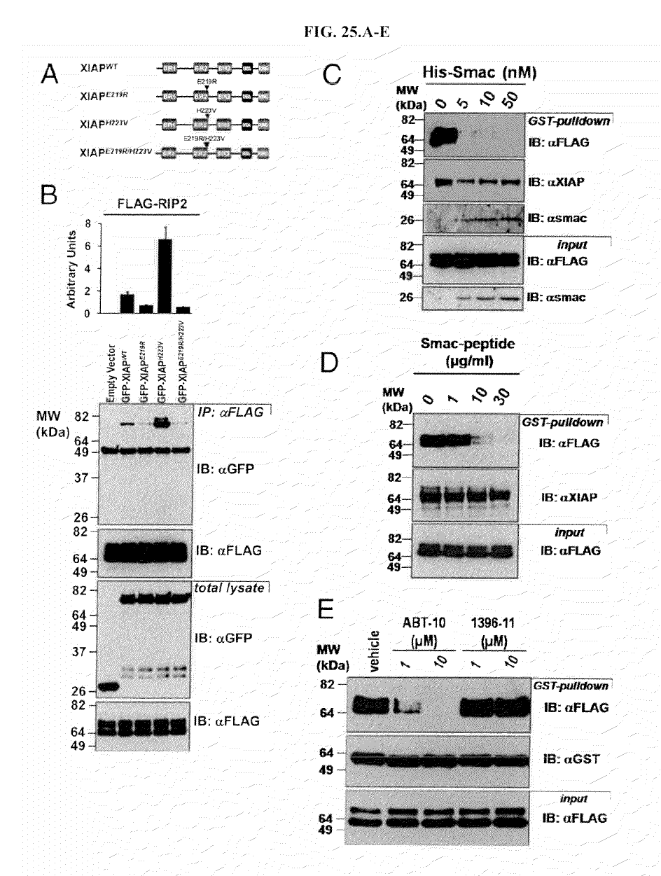

XIAP Binds RIP2 via the SMAC-Binding Site of BIR2. Because XIAP also binds SMAC/DIABLO via its BIR2 domain, binding assays were performed using XIAP constructs mutated at the SMAC-binding site of BIR2 (FIG. 25A). Previously, E219R and H223V mutations were shown to ablate SMAC binding to this domain, affecting critical residues for binding the Ala-Val-Ile-Pro tetrapeptide motif through which SMAC associates with a crevice on BIR2 (Scott et al. (2005)). With this in mind, HEK293T cells were transfected with FLAG-RIP2 and plasmids encoding GFP-fusion proteins containing wild-type versus mutant XIAP and co-IP experiments were performed. Interestingly, RIP2 showed decreased binding to the E219R XIAP mutant, whereas the H223V mutant showed increased binding to RIP2 compared with wild-type XIAP (FIG. 25B). In contrast, the XIAP mutants showed the expected SMAC binding properties, with the E219R and H223V mutations ablating SMAC protein binding to BIR2 but having no impact on SMAC binding via BIR3. Thus, mutation of residues in the same crevice on BIR2 that is involved in SMAC binding modulate binding to RIP2. Consistent with these observations, recombinant SMAC protein (but not control SseL protein) competed for XIAP binding to RIP2 in vitro, showing concentration-dependent inhibition of XIAP/RIP2 interaction at nanomolar concentrations (FIG. 25C). A synthetic peptide corresponding to the N-terminus of SMAC, which binds the aforementioned BIR2 crevice, also inhibited XIAP/RIP2 interaction in a concentration-dependent manner in vitro, although requiring micromolar concentrations (FIG. 25D), Note that SMAC protein is dimeric and binds both the BIR2 and BIR3 domains of XIAP, resulting in high affinity association via simultaneous two-side binding, whereas the peptide is monomeric (Liu et al. (2000)). Similarly, ABT-10, a small molecule compound that targets the SMAC-binding crevice on BIR domains, inhibited XIAP/RIP2 association in vitro (Oost et al. (2004)), whereas compound TPI-1396-11 that binds a non-SMAC site near BIR2 did not interfere with RIP2/XIAP association (Schimmer et al. (2004)) (FIG. 25E). When applied to cells, the ABT-10 compound also demonstrated inhibition of XIAP/RIP2 interaction, as assessed by co-IP experiments using lysates derived from the treated cells.

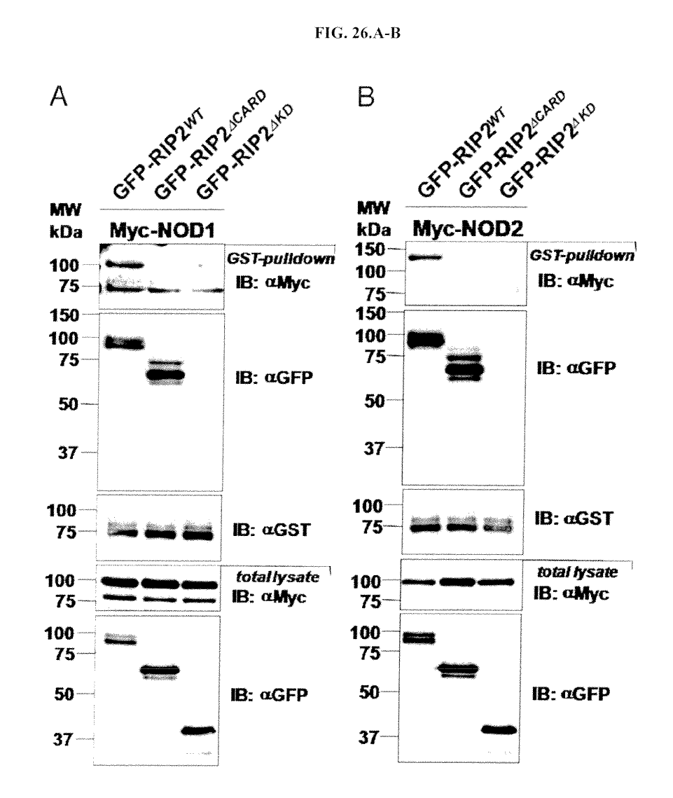

RIP2 Mediates Associates of XIAP with NOD1 and NOD2. RIP2 binds to NOD1 and NOD2 via a CARD-CARD interaction, whereas the results here indicate that XIAP binds to RIP2 independent of its CARD. Based on this, it was realized that RIP2 could molecularly bridge XIAP to the NOD1 and NOD2 complexes, by binding these proteins through different domains (kinase domain [KD] versus CARD). To establish this, recombinant GST-XIAP was used to pull down myc-NOD1 or myc-NOD2 produced by gene transfection in HEK293T cells and lysates in which RIP2 full-length protein or fragments of RIP2 were co-expressed were compared (FIGS. 26A and B). Expressing RIP2 resulted in clear pull-down of myc-NOD1 and myc-NOD2 with GST-XIAP. In contrast, neither RIP2ΔCARD nor RIP2ΔKD supported pull-down of myc-NOD1 or myc-NOD2 with GST-XIAP.

Finally, because the CARDs of NOD1 and NOD2 are required for binding RIP2 (Inohara et al. (1999), Ogura et al. (2001)), full-length NOD1/NOD2 and CARD deletion mutants of NOD1/2 were compared with respect to their ability to be pulled down by GST-XIAP. Whereas both full-length myc-NOD1 and myc-NOD2 were recovered from RIP2-containing lysates by GST-XIAP pull-down, the NOD1/NOD2 mutants with deletion of CARDs failed to associate with GST-XIAP. These results indicated that RIP2 serves as a bridge between XIAP and NOD1/NOD2.

### Discussion

In this example, evidence is presented that XIAP participates in NLR signaling by interacting with RIP2. The requirement for XIAP for NOD1 and NOD2-mediated activation of NF-κB was shown by studies of both homozygous XIAP gene knock-out cells and by using shRNA to knock-down XIAP expression. Furthermore, XIAP was found to be required when NF-κB induction was stimulated with either synthetic ligands that activate endogenous NOD1 and NOD2 or by gene transfer mediated over-expression of NOD1 and NOD2. In contrast, MAP deficiency did not impair the ability of other NF-κB inducers such as doxorubicin, PMA/ionomycin, and TNF-α to stimulate NF-κB activity. Thus, MAP participates selectively in the NF-κB pathway induced by NLR family members such as NOD1 and NOD2.

These results indicate that RIP2 serves as the link between XIAP and the NODs, where the CARD domain of RIP2 binds the CARDs of NOD1/NOD2 and the non-CARD regions (the kinase domain) of RIP2 binds XIAP. It was realized that XIAP provides a platform on which to assemble components of an IKK-activating complex, in as much as the BIR1 domain of XIAP binds the TAB/TAK complex, a known upstream activator of IKKs (Lu et al. (2007)). In this regard, RIP2 has been reported to hind the noncatalytic IKKgamma (NEMO) subunit of the IKK complex (Hasegawa et al. (2008)). Thus, with BIR2 of XIAP binding RIP2 (which binds IKKgamma) and BIR1 binding TAB/TAK (which phosphorylates IKKs), XIAP can bring the necessary components into close apposition for successful activation of IKKs and thus NF-κB.

Ubiquitination mediated by the RING domain of XIAP can also be a factor. In the case of RIP1, association with c-IAP1 or c-IAP2 (typically together with TRAFs) results in K63-linked ubiquitinylation of RIP1, a posttranslational modification that recruits TAB/TAK and a modification that also occurs on IKKgamma in the context of some pathways leading to NF-κB activation (Bertrand et al. (2008), Festjens et al. (2007)). Analogously, XIAP can interact with ubiquitin conjugating enzymes (e.g., UBC13) responsible for K63-linked phosphorylation when incorporated into NOD signalosomes, using its E3 ligase activity in facilitate IKK activation. It was realized that the participation of XIAP in NOD1/NOD2 signaling is reminiscent of the role of DIAP2 in innate immunity responses in Drosophila. In the fly, RNA interference screens have identified DIAP2 as an essential player in Drosophila innate immune signaling (Gesellchen et al. (2005), Huh et al. (2007), Leulier et al. (2006)). DIAP2 operates downstream of the PGRP-Lc receptor, in a signaling cascade involving IMD (fly ortholog of RIP1/RIP2), dTAB2, and dTAK1 that activates Rel (NF-κB) and JNK-dependent target gene expression (Gesellchen et al. (2005)).

The mutagenesis and competition experiments indicate that the SMAC binding crevice on the surface of BIR2 mediates interactions between XIAP and RIP2. In this regard, protein interactions involving this site include the proteolytically processed N terminus of SMAC and HtrA2/OMI and the processed N terminus of the small catalytic subunits of caspase-3 and -7 (Deveraux et al. (1997), Du et al. (2000), Suzuki et al. (2001)), in each case representing an N terminus created by proteolysis. The mutagenesis data also indicate that the residues lining the SMAC-binding crevice that contribute to RIP2 binding are at least partly different from those involved in SMAC and HtrA2/OMI binding, in as much as the H223V mutation inhibited SMAC but enhanced RIP2 binding. Because RIP2 could interact with XIAP BIR2 differently than SMAC interacts with BIR2, the SMAC-binding pocket could be allosterically regulating binding.

The observation that a chemical SMAC mimic inhibited XIAP/RIP2 association revealed another unanticipated function of these compounds. Recently, it was reported that SMAC-mimicking compounds binding c-IAP1 and c-IAP2 stimulate their E3 ligase activity, causing the destruction of these proteins and impacting the NF-κB signaling mechanism by causing the accumulation of NIK and altering regulation of RIP1 (Varfolomeev et al. (2007) The results here indicate that such compounds would inhibit NF-κB activity induced via the NOD-XIAP pathway, while simultaneously stimulating NF-κB via the aforementioned mechanisms involving NIK and possibly RIP1. These SMAC-mimicking compounds have multiple simultaneous cellular activities, which stimulate some NF-κB pathways (Varfolomeev et al. (2007), Vince et. al. (2007)), presumably inhibit other NF-κB pathways (e.g., NOD/XIAP), and induce apoptosis by dislodging caspases from BIR domains of IAP family proteins.

### REFERENCES

Abbott, D. W., Wilkins, A., Asara, J. M., and Cantley, L. C. The Crohn's disease protein, NOD2, requires RIP2 in order to induce ubiquitinylation of a novel site on NEMO. Curr Biol, 14: 2217-2227, 2004.
Bauler L D, Duckett C S, O'Riordan M X (2008) XIAP regulates cytosol-specific innate immunity to Listeria infection. PLoS Pathog 4:e1000142.
Bell J K, et al. (2003) Leucine-rich repeats and pathogen recognition in Toll-like receptors. Trends Immunol 24:528-533.
Bertin, J., Nir, W. J., Fischer, C. M., Tayber, O. V., Errada, P. R., Grant, J. R., Keilty, J. J., Gosselin, M. L., Robison, K. E., Wong, G. H., Glucksmann, M. A., and DiStefano, P. S. Human CARD4 protein is a novel CED-4/Apaf-1 cell death family member that activates NF-kappaB. J Biol Chem, 274: 12955-12958, 1999.
Bertrand M J, et al. (2008) cIAP1 and cIAP2 facilitate cancer cell survival by functioning as E3 ligases that promote RIP1 ubiquitination. Mol Cell 30:689-700.
Bertrand M J, et al. (2009) Cellular inhibitors of apoptosis cIAP1 and cIAP2 are required for innate immunity signaling by the pattern recognition receptors NOD1 and NOD2. Immunity.
Carneiro, L. A Travassos, L. H., and Girardin, S. E. Nod-like receptors in innate and inflammatory diseases. Ann Med, 39: 581-593, 2007.
Carneiro, L., Magalhaes, J., Tattoli, I., Philpott, D., and Travassos, L. Nod-like proteins in inflammation and disease. J Pathol, 214: 136-148, 2008.
Chamaillard, M. Hashimoto, M., Horie, Y., Masumoto, J., Qiu, S., Saab, L., Ogura, Y., Kawasaki, A., Fukase, K., Kusumoto, S., Valvano, M. A., Foster, S. J., Mak, T. W., Nunez, G., and Inohara, N. An essential role for NOD1 in host recognition of bacterial peptidoglycan containing diaminopimelic acid. Nat Immunol, 4: 702-707, 2003.
Cummins J M, et al. (2004) X-linked inhibitor of apoptosis protein (XIAP) is a nonredundant modulator of tumor necrosis factor-related apoptosis-inducing ligand (TRAIL)-mediated apoptosis in human cancer cells. Cancer Res 64:3006-3008.
da Silva Correia, J., Miranda, Y., Austin-Brown, N., Hsu, J., Mathison, J., Xiang, R., Zhou, H., Li, Q., Han, J., and Ulevitch, R. J. Nod1-dependent control of tumor growth. Proc Natl Acad Sci USA, 103: 1840-1845, 2006.
da Silva Correia, J., Miranda, Y., Leonard, N., Hsu, J., and Ulevitch, R. J. Regulation of Nod1-mediated signaling pathways. Cell Death Differ, 14: 830-839, 2007.
Deveraux Q L, et al. (1998) IAPs block apoptotic events induced by caspase-8 and cytochrome c by direct inhibition of distinct caspases. EMBO J 17:2215-2223.
Deveraux Q L, Takahashi R, Salvesen G S, Reed J C (1997) X-linked IAP is a direct inhibitor of cell-death proteases. Nature 388:300-304.
Du C, Fang M, Li Y, Li L, Wang X (2000) Smac, a mitochondrial protein that promotes cytochrome c-dependent caspase activation by eliminating IAP inhibition. Cell 102:33-42.
Duckett C S, et al. (1996) A conserved family of cellular genes related to the baculovirus iap gene and encoding apoptosis inhibitors. EMBO J 15:2685-2694.
Eckelman B P, Salvesen G S, Scott F L (2006) Human inhibitor of apoptosis proteins: Why XIAP is the black sheep of the family. EMBO Rep 7:988-994.
Faustin, B., Lartigue, L., Bruey, J. M., Luciano, F., Sergienko, E. Bailly-Maitre, B., Hannein, D., Rouiller, I., and Reed, J. C. Reconstituted NALP1 inflammasome reveals two-step mechanism of Caspase-1 activation. Molecular Cell, 25: 713-724, 2007.
Festjens N, Vanden Berghe T, Cornelis S, Vandenabeele P (2007) RIP1, a kinase on the crossroads of a cell's decision to live or die. Cell Death Differ 14:400-410.
Franchi, L., Park, J. H., Shaw, M. H., Marina-Garcia, N., Chen, G., Kim, Y. G., and Nunez, G. Intracellular NOD-like receptors in innate immunity, infection and disease. Cell Microbiol, 10:1-8, 2008.
Gesellchen V, Kuttenkeuler D, Steckel M, Pelte N. Boutros M (2005) An RNA interference screen identifies Inhibitor of Apoptosis Protein 2 as a regulator of innate immune signaling in Drosophila. EMBO Rep 6:979-984.
Girardin S E, et al. (2003a) Nod1 detects a unique muropeptide from gram-negative bacterial peptidoglycan, Science 300:1584-1587.
Girardin, S. E., Travassos, L. H., Herve, M., Blanot, D., Boneca, I. G., Philpott, D. J., Sansonetti, P. J., and Mengin-Lecreulx, D. Peptidoglycan molecular requirements allowing detection by Nod1 and Nod2. Biol Chem, 278: 41702-41708, 2003b.
Goetz. M. P., Toft, D. O., Ames, M. M., and Erlichman, C. The Hsp90 chaperone complex as a novel target for cancer therapy. Ann Oncol, 14: 1169-1176, 2003.
Gyrd-Hansen M, et al. (2008) IAPs contain an evolutionarily conserved ubiquitin-binding domain that regulates NF-kappaB as well as cell survival and oncogenesis. Nat Cell Biol 10:1309-1317.
Hahn, J. S. Regulation of Nod1 by Hsp90 chaperone complex. FEBS Lett, 579: 4513-4519, 2005.
Hasegawa, M., Fujimoto, Y., Lucas, P. C., Nakano, H., Fukase, K., Nunez, G., and Inohara, N. A critical role of RICK/RIP2 polyubiquitination in Nod-induced NF-kappaB activation. Embo J, 27:373-383, 2008.
Hugot. J. P., Chamaillard, M., Zouali, H., Lesage, S., Cezard, J. P., Belaiche, J., Almer, S., Tysk, C., O'Morain, C. A., Gassull, M., Binder, V., Finkel, Y., Cortot, A., Modigliani, R., Laurent-Puig, P., Gower-Rousseau, C., Macry, J., Colombel, J. F., Sahbatou, M., and Thomas, G. Association of NOD2 leucine-rich repeat variants with susceptibility to Crohn's disease. Nature. 411: 599-603, 2001.
Huh J R, et al. (2007) The Drosophila inhibitor of apoptosis (IAP) DIAP2 is dispensable for cell survival, required for the innate immune response to gram-negative bacterial infection, and can be negatively regulated by the reaper/hid/grim family of IAP-binding apoptosis inducers. J Biol Chem 282:2056-2068.
Inohara N, et al. (2003) Host recognition of bacterial muramyl dipeptide mediated through NOD2. Implications for Crohn's disease. J Biol Chem 278:5509-5512.
Inohara, Chamaillard, McDonald, C., and Nunez, G. NOD-LRR proteins: role in host-microbial interactions and inflammatory disease. Annu Rev Biochem, 74: 355-383, 2005.
Inohara, N., Koseki, T., del Peso, L., Hu, Y., Yee, C., Chen, S., Carrio, R., Merino, J., Liu, D., Ni, J. and Nunez, G. Nod1, an Apaf-1-like activator of caspase-9 and nuclear factor-kappaB. J Biol Chem, 274: 14560-14567, 1999.
Kanneganti, T. D., Lamkanfi, M. and Nunez, G. Intracellular NOD-like receptors in host defense and disease. Immunity, 27: 549-559, 2007.
Kobayashi K, et al. (2002) RICK/Rip2/CARDIAK mediates signaling for receptors of the innate and adaptive immune systems. Nature 416:194-199.
Krieg A, Le Negrate G, Reed J C (2009) RIP2-beta: A novel alternative mRNA splice variant of the receptor interacting protein kinase RIP2. Mol Immunol 46:1163-1170.
Lala. S., Ogura, Y., Osborne, C., Hor, S. Y., Bromfield, A., Davies, S., Ogunbiyi, O., Nunez. G., and Keshav. S. Crohn's disease and the NOD2 gene: a role for paneth cells. Gastroenterology, 125: 47-57, 2003.
Leulier F. Lhocine N, Lemaitre B, Meier P (2006) The Drosophila inhibitor of apoptosis protein DIAP2 functions in innate immunity and is essential to resist Gram-negative bacterial infection. Mol Cell Biol 26:7821-7831.
Li, Q. and Verma, I. M. NF-kappaB regulation in the immune system. Nat Rev Immunol, 2: 725-734, 2002.
Liu Z, et al. (2000) Structural basis for binding of Smac/DIABLO to the XIAP BIR3 domain. Nature 408:1004-1008.
Lu M. et al. (2007) XIAP induces NF-kappaB activation via the BIR1/TAB1 interaction and BIR1 dimerization. Mol Cell 26:689-702.
Maeda, S., Hsu, L. C., Liu, H., Bankston, L. A., Iimura, M., Kagnoff, M. F., Eckmann, L., and Karin, M. Nod2 mutation in Crohn's disease potentiates NF-kappaB activity and IL-1 beta processing. Science, 307: 734-738, 2005.
Matsuzawa S I, Reed J C (2001) Siah-1, SIP, and Ebi collaborate in a novel pathway for beta-catenin degradation linked to p53 responses. Mol Cell 7:915-926.
McCarthy J V, Ni J, Dixit V M (1998) RIP2 is a novel NF-kappaB-activating and cell death inducing kinase. J Biol Chem 273:16968-16975.
Murray, P. J. NOD proteins: an intracellular pathogen-recognition system or signal transduction modifiers? Curr Opin Immunol, 17: 352-358, 2005.
Naldini L, Blower U, Gage F H, Trono D, Verma I M (1996) Efficient transfer, integration, and sustained long-term expression of the transgene in adult rat brains injected with a lentiviral vector. Proc Natl Acad Sci USA 93:11382-11388.
Ogura, Y., Inohara, N., Benito, A., Chen, F. F., Yamaoka, S., and Nunez, G. Nod2, a Nod1/Apaf-1 family member that is restricted to monocytes and activates NF-kappaB. J Biol Chem, 276: 4812-4818, 2001.
Oost T K, et al. (2004) Discovery of potent antagonists of the antiapoptotic protein XIAP for the treatment of cancer. J Med Chem 47:4417-4426.
Opitz B, et al. (2004) Nucleotide-binding oligomerization domain proteins are innate immune receptors for internalized Streptococcus pneumoniae. J Biol Chem 779:36426-36432.
Perkins, N. D. Integrating cell-signalling pathways with NF-kappaB and IKK function. Nat Rev Mol Cell Biol, 8: 49-62, 2007.
Pfeifer, A. and Verma. I. M. Gene therapy: promises and problems. Annu Rev Genomics Hum Genet, 2: 177-211, 2001.
Pfeifer, A. Ikawa, M., Dayn, Y., and Verma, I. M. Transgenesis by lentiviral vectors: lack of gene silencing in mammalian embryonic stein cells and preimplantation embryos. Proc Natl Acad Sci S A, 99: 2140-2145, 2002.
Roy N, Deveraux Q L, Takahashi R, Salvesen G S, Reed J C (1997) The c-IAP-1 and c-IAP-2 proteins are direct inhibitors of specific caspases. EMBO J 6:6914-6925.
Sanna M G, Duckett C S, Richter B W, Thompson C B, Ulevitch R J (1998) Selective activation of JNK1 is necessary for the anti-apoptotic activity of hILP. Proc Natl Acad Sci USA 95:6015-6020.
Schimmer A D, et al. (2004) Small-molecule antagonists of apoptosis suppressor XIAP exhibit broad antitumor activity. Cancer Cell 5:25-35.
Scott F L, et al. (2005) XIAP inhibits caspase-3 and -7 using two binding sites: Evolutionarily conserved mechanism of IAPs. EMBO J 24:645-655.
Sun C, et al. (1999) NMR structure and mutagenesis of the inhibitor-of-apoptosis protein XIAP. Nature 401:818-822.
Suzuki Y, et al. (2001) A serine protease, HtrA2, is released from the mitochondria and interacts with XIAP, inducing cell death. Mol Cell 8:613-621.
Tattoli, I., Travassos, L. H., Carneiro, L. A., Magalhaes, J. G., and Girardin, S. E. The Nodosome Nod1 and Nod2 control bacterial infections and inflammation. Semin Immunopathol, 29: 289-301, 2007.
Thome M, et al. (1998) Identification of CARD AK, a RIP-like kinase that associates with caspase-1. Curr Biol 8:885-888.
Tiscornia G, Singer O, Ikawa M, Verma I M (2003) A general method for gene knockdown in mice by using lentiviral vectors expressing small interfering RNA. Proc Natl Acad Sci USA 100:1844-1848.
Uehara, A., Fujimoto, Y., Fukase, K., and Takada, H. Various human epithelial cells express functional Toll-like receptors. NOD1 and NOD2 to produce anti-microbial peptides, but not proinflammatory cytokines. Mol Immunol, 44: 3100-3111, 2007.
Ulevitch, R. J. Therapeutics targeting the innate immune system. Nat Rev Immunol, 4: 512-520, 2004.
Varfolomeev E, et al. (2007) IAP antagonists induce autoubiquitination of c-IAPs, NFkappaB activation, and TNFalpha-dependent apoptosis. Cell 131:669-681.
Viala, J., Sansonerti, P., and Philpott, D. J. Nods and ‘intracellular’ innate immunity. C R Biol, 327: 551-555, 2004.
Vince J E, et al. (2007) IAP antagonists target cIAP1 to induce TNFalpha-dependent apoptosis. Cell 131:682-693.
Yang Y, Fang S, Jensen J P, Weissman A M, Ashwell J D (2000) Ubiquitin protein ligase activity of IAPs and their degradation in proteasomes in response to apoptotic stimuli. Science 288:874-877.
Yoo. N. J., Park, W. S., Kim, S. Y., Reed, J. C., Son, S. G., Lee, J. Y., and Lee, S. H. Nod1, a CARD protein, enhances pro-interleukin-1beta processing through the interaction with pro-caspase-1. Biochem Biophys Res Commun, 299: 652-658, 2002.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

We claim:

1. A method of modulating NOD1 biological activity comprising contacting NOD1 with a compound of Formula III: 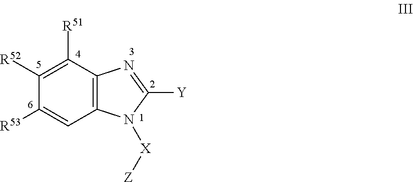
or a pharmaceutically acceptable salt thereof, wherein
R⁵¹ is H, C₁-C₆ alkyl, C₃-C₆ cycloalkyl, or NH₂;
R⁵² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or fluoro;
R⁵³ is H, C₁-C₆ alkyl, C₁-C₆ alkyl substituted with fluoro, C₃-C₆ cycloalkyl, C₃-C₆ cycloalkyl substituted with fluoro, halo, CO₂H, or a carboxyl ester;
Y is NH₂, H, NH(CH₂)₃OH, CH₃, or —CH₂NHCHO;
X is SO₂, CO, —CH₂—, or —CH₂CH₂CO—;
Z is: 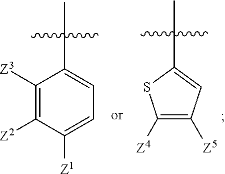
Z¹ is H, NO₂, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₁-C₆ alkoxy substituted with fluoro, C₃-C₆ cycloalkoxy, C₃-C₆ cycloalkoxy substituted with fluoro, halo, or is 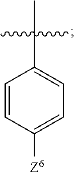
Z⁶ is H, C₁-C₃ alkoxy, C₃-C₄ cycloalkoxy, or halo;
Z² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or halo, or Z¹ and Z² together with the carbon atoms they are attached to form a 5-7 membered ring;
Z³ is H or halo;
Z⁴ and Z⁵ are independently H or halo, or Z⁴ and Z⁵ together with the carbon atoms they are attached to form a 5-7 membered ring.

2. The method of claim 1, wherein the NOD1 biological activity is NF-κB activation, IRF activation, stress kinase activation, autophagy stimulation, or inflammatory activation of caspase-1, -4, or -5.

3. The method of claim 1, wherein R⁵³ is H.

4. The method of claim 1, wherein R⁵² and R⁵³ are H.

5. The method of claim 1, wherein R⁵¹ and R⁵³ are H.

6. The method of claim 1, wherein R⁵¹ and R⁵² are H.

7. The method of claim 1, wherein R⁵¹, R⁵² and R⁵³ are H.

8. The method of any one of claims 1-7, wherein X is SO₂.

9. The method of any one of claims 1-8, wherein Y is NH₂.

10. The method of any one of claims 1-9, wherein Z is:

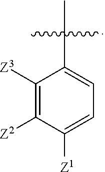

11. The method of claim 10, wherein Z¹ is H, NO₂, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₁-C₆ alkoxy substituted with fluoro, C₃-C₆ cycloalkoxy, C₃-C₆ cycloalkoxy substituted with fluoro, or halo.

12. The method of claim 10, wherein Z² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or halo.

13. The method of claim 10, wherein Z³ is H.

14. The method of claim 1, wherein Z is:

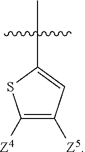

15. The method of claim 1, wherein
R¹, R² and R³ are H;
Y is NH₂;
X is SO₂;
Z is: 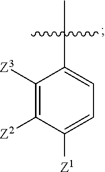
Z¹ is H, C₁-C₆ alkyl, C₁-C₆ alkyl substituted with fluoro, C₁-C₆ alkoxy, or halo;
Z² is H, C₁-C₆ alkyl, C₁-C₆ alkoxy, or halo, or Z₁ and Z₂ together with the carbon atoms they are attached to form a 5 membered ring containing carbon ring atoms; and
Z³ is H.

16. The method of claim 15, wherein Z¹ is H, methyl, isopropyl, trifluoromethyl, methoxy, or chloro.

17. The method of claim 15, wherein
Z² is H, propyl, tertiary butyl, methoxy, or halo.

18. The method of claim 1, wherein
R⁵¹, R⁵² and R⁵³ are H;
Y is NH₂;
X is SO₂;
Z is: 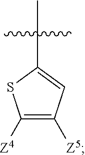
and
Z⁴ and Z⁵ together with the carbon atoms they are attached to form an aromatic ring.

19. The method of claim 1, wherein
R⁵³ is H;
at least one of R⁵¹ and R⁵² is a non hydrogen substituent;
Y is NH₂;
X is SO₂;
Z is: 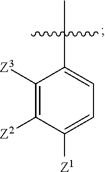
Z¹ is C₁-C₆ alkyl, C₃-C₆ cycloalkyl; and
Z² and Z³ are H.

20. The method of claim 1, wherein
R⁵¹, R⁵² and R⁵³ are H;
Y is NH₂;
X is SO₂;
Z is: 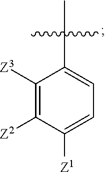
and
Z² and Z³ are H.

21. The method of any one of claims 1-20, wherein the contacting performed in vitro or in vivo.

22. The method of any one of claims 1-20, wherein the contacting is performed in vitro.

23. The method of any one of claims 1-20, wherein the contacting is performed in vivo.

24. A pharmaceutical composition comprising a pharmaceutically acceptable excipient and a therapeutically effective amount of a compound of Formula III: 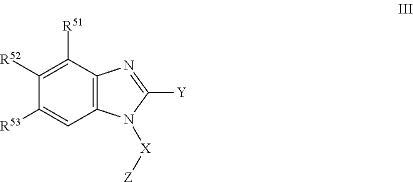
or a pharmaceutically acceptable salt thereof, wherein
R⁵¹ is H, C₁-C₆ alkyl, C₃-C₆ cycloalkyl, or NH₂;
R⁵² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or fluoro;
R⁵³ is H, C₁-C₆ alkyl, C₁-C₆ alkyl substituted with fluoro, C₃-C₆ cycloalkyl, C₃-C₆ cycloalkyl substituted with fluoro, halo, CO₂H, or a carboxyl ester;
Y is NH₃, H, NH(CH₂)₃OH, CH₃, or —CH₂NHCHO;
X is SO₂, CO, —CH₂—, or —CH₂CH₂CO—;
Z is: 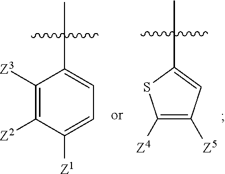
Z¹ is H, NO₂, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₁-C₆ alkoxy substituted with fluoro, C₃-C₆ cycloalkoxy, C₃-C₆ cycloalkoxy substituted with fluoro, halo, or is 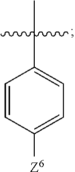
Z⁶ is H, C₁-C₃ alkoxy, C₃-C₄ cycloalkoxy, or halo;
Z² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or halo, or Z¹ and Z² together with the carbon atoms they are attached to form a 5-7 membered ring;
Z³ is H or halo;
Z⁴ and Z⁵ are independently H or halo, or Z⁴ and Z⁵ together with the carbon atoms they are attached to form a 5-7 membered ring.

25. A method for treating a patient diagnosed as having a disease selected from the group consisting of inflammatory diseases, infectious diseases, neurodegenerative disease, cardiovascular disease, sepsis, and diabetes-related diseases comprising administering to the patient a therapeutically effective amount of a compound of Formula III, wherein the compound of formula III has the structure: 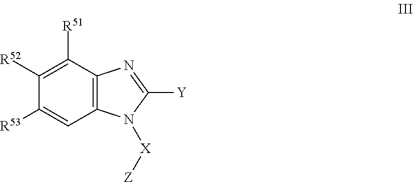
or a pharmaceutically acceptable salt thereof, wherein
R⁵¹ is H, C₁-C₆ alkyl, C₃-C₁, cycloalkyl, or NH₂;
R⁵² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or fluoro;
R⁵³ is H, C₁-C₆ alkyl, C₁-C₆ alkyl substituted with fluoro, C₃-C₆ cycloalkyl, C₃-C₆ cycloalkyl substituted with fluoro, halo, CO₂H, or a carboxyl ester;
Y is NH₂, H, NH(CH₂)₃OH, CH₃, or —CH₂NHCHO;
X is SO₂, CO, —CH₂—, or —CH₂CH₂CO—;
Z is: 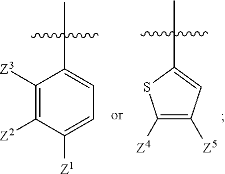
Z¹ is H, NO₂, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₁-C₆ alkoxy substituted with fluoro, C₃-C₆ cycloalkoxy, C₃-C₆ cycloalkoxy substituted with fluoro, halo, or is 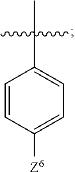
Z⁶ is H, C₁-C₃ alkoxy, C₃-C₄ cycloalkoxy or halo;
Z² is H, C₁-C₆ alkyl, C₁-C₆ alkyl substituted with fluoro, C₃-C₆ cycloalkyl, C₃-C₆ cycloalkyl substituted with fluoro, C₁-C₆ alkoxy, C₃-C₆ cycloalkoxy, or halo, or Z¹ and Z² together with the carbon atoms they are attached to form a 5-7 membered ring;
Z³ is H or halo;
Z⁴ and Z⁵ are independently H or halo, or Z⁴ and Z⁵ together with the carbon atoms they are attached to form a 5-7 membered ring.

26. The method of claim 25, wherein the disease is an inflammatory disease associated with Type I, Type II, Type III, Type IV, Type V, or delayed hypersensitivity.

27. A method of identifying potential modulators of NOD1, NOD2, or both, the method comprising:
(a) bringing into contact a test compound and a NOD test cell, wherein the NOD test cell is a mammalian cell comprising an NF-κB-responsive reporter construct, wherein the reporter is expressed under NOD-inducing conditions, wherein the NOD test cell is exposed to NOD-inducing conditions,
(b) detecting the level of expression of the reporter, wherein a level of expression of the reporter above or below a control level of expression of the reporter indicates that the test compound is a potential modulator of NOD1, NOD2, or both, wherein the control level of expression of the reporter is the level of expression of the reporter when the NOD test cell is exposed to the NOD-inducing conditions in the absence of any test compound.

28. The method of claim 27 further comprising:
(c) bringing into contact the test compound, a NOD inducer, and an IL-8 test cell, wherein the IL-8 test cell is a second mammalian cell comprising a NOD expression construct, wherein NOD1, NOD2, or both is expressed from the NOD expression construct,
(d) detecting the level of Interleukin-8 (IL-8) produced by the IL-8 test cell, wherein a level of IL-8 above or below a control level of IL-8 further indicates that the test compound is a potential modulator of NOD1, NOD2, or both, wherein the control level of IL-8 is the level of IL-8 when the IL-8 test cell is exposed to the NOD inducer under the same conditions but in the absence of any test compound.

29. A method of identifying potential modulators of NOD1, NOD2, or both, the method comprising:
(a) bringing into contact a test compound and a NOD test cell, wherein the NOD test cell is a mammalian cell comprising an ISGE-responsive reporter construct, wherein the reporter is expressed under NOD-inducing conditions, wherein the NOD test cell is exposed to NOD-inducing conditions,
(b) detecting the level of expression of the reporter, wherein a level of expression of the reporter above or below a control level of expression of the reporter indicates that the test compound is a potential modulator of NOD1, NOD2, or both, wherein the control level of expression of the reporter is the level of expression of the reporter when the NOD test cell is exposed to the NOD-inducing conditions in the absence of any test compound.

30. A method of identifying potential modulators of NOD1, NOD2, or both, the method comprising:
(a) bringing into contact a test compound and a NOD test cell, wherein the NOD test cell is a mammalian cell comprising an AP-1-responsive reporter construct, wherein the reporter is expressed under NOD-inducing conditions, wherein the NOD test cell is exposed to NOD-inducing conditions,
(b) detecting the level of expression of the reporter, wherein a level of expression of the reporter above or below a control level of expression of the reporter indicates that the test compound is a potential modulator of NOD1, NOD2, or both, wherein the control level of expression of the reporter is the level of expression of the reporter when the NOD test cell is exposed to the NOD-inducing conditions in the absence of any test compound.

31. The method of claim 29 or 30 further comprising:
(c) bringing into contact the test compound, a NOD inducer, and an Interferon test cell, wherein the Interferon test cell is a second mammalian cell comprising a NOD expression construct, wherein NOD1, NOD2, or both is expressed from the NOD expression construct,
(d) detecting the level of Interferon produced by the Interferon test cell, wherein a level of Interferon above or below a control level of Interferon further indicates that the test compound is a potential modulator of NOD1, NOD2, or both, wherein the control level of Interferon is the level of Interferon when the Interferon test cell is exposed to the NOD inducer under the same conditions but in the absence of any test compound.
